(19) United States
(12) Patent Application Publication
(10) Pub. No.: US 2026/0224544 A1
(43) Pub. Date: Aug. 6, 2026

(54) Intranasal Formulations for Treating Obstructive Sleep Apnea

(71) Applicant: Mosanna Therapeutics AG, Basel (CH)

(72) Inventors: Marc Barry BROWN, Watford (GB); Paul Michael David HILL, Farnham (GB); Alisha Taya ALLY, Guildford (GB); Charles Rodey Greenaway EVANS, Godalming (GB); Jonathan Peter TALBOT, Füllinsdorf (CH); Sandrien JANSSENS, Rotselaar (BE)

(21) Appl. No.: 19/631,461

(22) Filed: Mar. 27, 2026

Related U.S. Application Data
(63) Continuation of application No. PCT/EP2024/077436, filed on Sep. 30, 2024.
(60) Provisional application No. 63/586,254, filed on Sep. 28, 2023.
(60) Provisional application No. 63/599,900, filed on Nov. 16, 2023.

(30) Foreign Application Priority Data
Oct. 17, 2023 (GB) 2315863.7

(51) Int. Cl. A61K 31/4406; A61K 9/00; A61K 47/18; A61K 47/26; A61K 47/38

(52) U.S. Cl. CPC A61K 31/4406; A61K 9/0043; A61K 47/186; A61K 47/26; A61K 47/38

(57) ABSTRACT

The present disclosure relates to nasal pharmaceutical compositions for the treatment of obstructive sleep apnea, drug-device combinations for intranasal delivery of the pharmaceutical compositions, and methods of treatment.

### 1. BACKGROUND OF THE INVENTION

Obstructive sleep apnea (OSA), also known as obstructive sleep apnea-hypopnea syndrome (OSAHS), is estimated to affect one third of the adult population with an estimated global prevalence of almost one billion. Obstructive sleep apnea leads to daily debility, including excessive daytime sleepiness, and over the longer term increases the risk of a variety of adverse sequelae, including hypertension and cardiometabolic syndromes.

Treatment of OSA has changed little over the past several decades, with the overwhelming majority of patients treated with positive airway pressure, primarily continuous positive airway pressure (CPAP). Compliance with CPAP is poor. Oral appliances to advance the mandible during sleep have poor compliance and uncertain efficacy. Surgical approaches to open the airway—such as surgical removal of oral or oropharyngeal soft tissue and maxillomandibular advancement surgery—are invasive, expensive, and are not appropriate for all patients' anatomy.

There is, therefore, a need for new therapies for reducing severity of OSA.

Intranasal administration of potassium channel blockers to increase activity of pharyngeal dilator muscles, notably the genioglossus muscle, has been suggested as a possible pharmacologic approach to treating OSA. In 2013, Wirth and colleagues reported that the potassium channel blocker AVE0118 (2′-{[2-(4-Methoxy-phenyl)-acetylamino]-methyl}-biphenyl-2-carboxylic acid (2-pyridin-3-yl-ethyl)-amide (compound 1)), administered topically to the upper airway of anesthetized pigs, was able to sensitize the negative pressure reflex (NPR), shifting the mechanoreceptor response threshold for the genioglossus muscle to more positive pressures and dose-dependently inhibit upper airway collapsibility (Wirth et al., SLEEP, 36(5):699-708 (2013)). No human clinical studies of compound 1 in OSA have been published. However, Gaisl et al., Eur. Respir. J 58:2101937 (2021), describe a phase 2 clinical trial testing intranasal administration of a potassium channel blocker, BAY 2253651, but concluded that a single dose applied nasally did not lead to a reduction in the apnea hypoxia index (AHI) in people with moderate to severe OSA off CPAP. Osman et al., Chest 163(4):953-965 (2023), recently described a small clinical trial of 12 selected patients with a different K⁺ blocker, BAY 2586116, reporting that intranasal administration of BAY 2586116 improved pharyngeal collapsibility by an average of approximately 2 cm H₂O versus placebo in patients with OSA, which falls well short of the 4-6 cm change achievable with dental appliances, and Bayer is no longer actively developing BAY 2586116 (bayer.com/sites/default/files/ph-rd-pipeline-2023-08-final-new.pdf).

There is a continued need for pharmacologic agents capable of treating OSA.

### 2. SUMMARY OF THE INVENTION

Notwithstanding the reported failures of intranasal potassium channel blockers in the decade since Wirth's original report, a phase 2 clinical trial of intranasal administration of AVE-118 (compound 1) was conducted in human patients with OSA. As reported in Example 1 herein, this trial failed to meet its primary efficacy endpoint, demonstrating no significant improvement in obstructive sleep apnea after intranasal administration of compound 1.

Among the many conceivable reasons that the trial failed, the possibility that Wirth's pig model lacks translational relevance to human OSA would have precluded further development, given the absence of in vitro tests capable of modeling (i) adequacy of nasopharyngeal deposition of the channel blocker proximal to genioglossal nerve endings, with subsequent (ii) entry into cells, and then (iii) 8 hour persistence at the nerve endings. However, as described in Example 3, we were able to demonstrate translational relevance of the Wirth pig model.

Using a modification of the Wirth pig OSA model, we could then test whether failure of the phase 2 trial could instead be attributed to use of a suspension formulation of compound 1 in the trial. With the pig in a vertical position, intranasal administration of 0.3 mg of compound 1 in a solution formulation fully inhibited upper airway collapsibility, with greater duration of efficacy than that provided by the phase 2 suspension formulation. See Example 4. However, when intranasal administration of compound 1 was followed by nasal lavage to model the continuous flow of mucus during recumbency and sleep, the effect of compound 1 in solution formulation was quickly and completely abolished. See Example 5.

Although both suspension and solution formulations had failed to provide 8-hour reduction in upper airway collapsibility in the pig model, we discovered that microparticulate suspensions of compound 1 in formulations containing various mucoadhesive polymers allowed effective treatment OSA in the vertical pig model, with the ability to reduce upper airway collapsibility resistant to post-administration nasal lavage. See Example 6. These properties make possible treatment of OSA by intranasal administration of potassium channel blockers, in particular pan-K channel inhibitors such as compound 1.

Accordingly, in one aspect, the present disclosure provides pharmaceutical compositions comprising 2′-{[2-(4-Methoxy-phenyl)-acetylamino]-methyl}-biphenyl-2-carboxylic acid (2-pyridin-3-yl-ethyl)-amide (compound 1) or a pharmaceutically acceptable salt thereof and a mucoadhesive polymer.

In another aspect, the present disclosure provides a method of treating obstructive sleep apnea in a subject in need thereof comprising administering an effective amount of the pharmaceutical composition described herein to the subject prior to sleep. In certain embodiments, the composition is administered intranasally by a drug device-combination product comprising a nasal delivery device and one or more doses of the pharmaceutical composition described herein.

In another aspect, the present disclosure provides a pharmaceutical composition as described herein for use in a method of treating obstructive sleep apnea in a subject in need thereof, wherein said method comprises administering an effective amount of the pharmaceutical composition described herein to the subject prior to sleep. In certain embodiments, the composition is administered intranasally by a drug device-combination product comprising a nasal delivery device and one or more doses of the pharmaceutical composition described herein.

### 3. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a schematic representation of the study design for the phase 2 clinical trial described in Example 1 below.

FIG. 2 plots mean change from baseline in apnea hypoxia index (AHI) (+/−95% confidence interval) in the per-protocol (PP) population of the clinical trial described in Example 1.

FIG. 3 is a plot of individual values of AHI in the per protocol (PP) population in the clinical trial described in Example 1.

FIG. 4 shows the effect of IV administration of a 20 μg/kg bolus and 12.5 μg/kg/h infusion of oxybutynin followed by IV administration of 1 mg/kg atomoxetine bolus and 275 μg/kg/h infusion for 4 h on upper airway collapsibility in the anesthetized pig model.

FIG. 5 shows the effect of nasal administration of 0.3 mg (400 μl per nostril) of the compound 1 solution formulation in Example 4 on upper airway collapsibility in the anesthetized pig model.

FIG. 6 shows the effect of nasal administration of 12 mg (200 μl/nostril) of the compound 1 suspension formulation in Example 4 (3% w/w) on upper airway collapsibility in the anesthetized pig model.

FIG. 7 shows the effect of nasal lavage following nasal administration of 0.3 mg (400 μl per nostril) of the compound 1 solution formulation in Example 4 on upper airway collapsibility in the anesthetized pig model.

FIG. 8 shows the effect of nasal lavage following nasal administration of 3 mg (400 μl per nostril) of the compound 1 solution formulation in Example 4 on upper airway collapsibility in the anesthetized pig model.

FIG. 9 shows the effect of nasal lavage following nasal administration of 24 mg (400 μl/nostril) of the compound 1 suspension formulation in Example 4 (3% w/w) on upper airway collapsibility in the anesthetized pig model.

FIG. 10 shows the effect of nasal lavage following nasal administration of 24 mg (400 μl/nostril) of the compound 1 suspension formulation in Example 4 (3% w/w) on upper airway collapsibility in the anesthetized pig model.

FIG. 11 shows the effect of nasal lavage following nasal administration of 18 mg (400 μl/nostril) of the compound 1 suspension formulation in Example 4 (2.25% w/w) on upper airway collapsibility in the anesthetized pig model.

FIG. 12 shows the average time to onset of action for each of the formulations of compound 1 tested in the anesthetized pig model at each of three tested negative pressure levels: −50 cm, −100 cm, and −150 cm H₂O.

FIG. 13 shows the average duration of action for each of the formulations of compound 1 tested in the anesthetized pig model at each of three tested negative pressure levels: −50 cm, −100 cm, and −150 cm H₂O. The observation period ended at 480 minutes.

FIGS. 14-21 show individual pig data for time to onset of action and duration of action for the formulations of compound 1 tested in the anesthetized pig model at each of three tested negative pressure levels: −50 cm, −100 cm, and −150 cm H₂O.

FIG. 22 shows an exemplary nasal device used to delivery compositions of compound 1. The fluid dispenser pump of the device is in the resting position.

FIG. 23 shows an exemplary nasal device used to delivery compositions of compound 1. The fluid dispenser pump of the device is in the actuating position.

FIG. 24 shows representative microscopic images of the Phase 2 formulation (3% compound 1) at t=0 (100× and 400×).

FIG. 25 shows representative microscopic images of SU28 (9% (w/w) compound 1) and SU41 (9% (w/w) compound 1) at t=0 and t=4 weeks following 4 weeks of storage at 40° C. (400×).

FIG. 26 shows representative microscopic images of SU40 (9% (w/w) compound 1) and SU45 (9% (w/w) compound 1) at t=0 and t=4 weeks following 4 weeks of storage at 25° C. and 40° C. (100×).

FIG. 27 shows sedimentation rates for (left to right) SU28, SU41, SU40, and SU45. All formulations contain 9% (w/w) compound 1.

FIG. 28 shows the study design for the scintigraphy study of Example 8. TV1=treatment visit 1 (SU28₁₀); TV2=treatment visit 2 (SU45₁₀); TV3=treatment visit 3 (SU41₁₀); and TV4=treatment visit 4 (SU45₃₀). All formulations contain 9% (w/w) compound 1.

### 4. DETAILED DESCRIPTION OF THE INVENTION

### 4.1. Definitions

When describing the embodiments of the present disclosure, the following terms, if present, have the following meanings, unless otherwise indicated. If not otherwise defined, terms have their customary meaning in the relevant art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, the term “effective amount,” means a sufficient amount of the compound or composition to provide the desired utility when administered to a subject. The term “effective amount” therefore refers to an amount of a compound or composition that is sufficient to promote a particular effect when administered to a subject in need of treatment. In certain embodiments, an effective amount includes an amount of compound or composition sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation. For example, when administered in clinic, such compounds or compositions will contain an amount of active ingredient effective to achieve the desired result.

As used herein, “mucoadhesive” refers to the ability of a substance (e.g., a polymer) or composition to adhere to a mucous membrane for an extended period of time.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium, and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “subject” refers to the person or organism to which the composition is, or is intended to be, administered. As such, subjects of the invention may include but are not limited to mammals, e.g., humans and other primates, such as chimpanzees and other ape and monkey species. In preferred embodiments the subject are humans. The term subject includes a person or organism of any age, weight, or other physical characteristic, including an adult, an adolescent, a child, an infant or a newborn.

As used herein, “treating” or “treatment” have the broadest meaning commonly accepted in the art of sleep apnea and hypopnea treatment, and therefore does not require cure or remission. The terms include, for example, a suppression or an amelioration of the symptoms associated with the condition afflicting the subject, where suppression and amelioration are used in a broad sense to refer to at least a reduction in the magnitude of a parameter, e.g., symptom, associated with the condition being treated, such as obstructive sleep apnea. As such, treatment also includes situations where the condition is completely inhibited, e.g., prevented from happening, or stopped, e.g., terminated, such that the subject no longer experiences the condition. As such, treatment includes both preventing and managing a condition.

In typical embodiments, the present disclosure is intended to encompass the compounds disclosed herein, and the pharmaceutically acceptable salts, pharmaceutically acceptable esters, tautomeric forms, polymorphs, and prodrugs of such compounds. In certain embodiments, the present disclosure includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. The present disclosure additionally encompasses compounds described herein as individual isomers substantially free of other isomers, and alternatively, as mixtures of various isomers.

### 4.2. Compositions

In one aspect, the present disclosure provides pharmaceutical compositions comprising 2′-{[2-(4-methoxy-phenyl)-acetylamino]-methyl}-biphenyl-2-carboxylic acid (2-pyridin-3-yl-ethyl)-amide (compound 1) or a pharmaceutically acceptable salt thereof having the following structure:

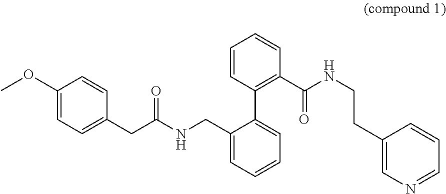 [SMILES: COc1ccc(CC(=O)NCc2ccccc2-c2ccccc2C(=O)NCCc2cccnc2)cc1]

The molecular formula of compound 1 is C₃₀H₂₉N3O₃.

The compositions described herein can be used in the treatment of sleep disorders. e.g., obstructive sleep apnea (OSA) and obstructive sleep apnea-hypopnea syndrome (OSAHS). The present disclosure provides compositions comprising compound 1 and a mucoadhesive polymer.

In certain embodiments, the pharmaceutical compositions provided herein are in the form of a liquid. In certain embodiments, the pharmaceutical composition is a solution. In certain embodiments, the pharmaceutical composition is a suspension. In certain embodiments, the pharmaceutical composition is a microparticulate suspension.

The amount of compound 1 in the composition can vary. In certain embodiments, the composition comprises compound 1 in an amount of 0.1% (w/w) or greater, such as, for example, 0.2% (w/w) or greater, 0.3% (w/w) or greater, 0.4% (w/w or greater), 0.5% (w/w) or greater, 0.6% (w/w) or greater, 0.7% (w/w) or greater, 0.9% (w/w) or greater, 1% (w/w) or greater, 1.25% (w/w) or greater, 1.5% (w/w) or greater, 1.75% (w/w) or greater, 2% (w/w) or greater, 2.25% (w/w) or greater, 2.5% (w/w) or greater, 2.75% (w/w) or greater, 3% (w/w) or greater, 3.25% (w/w) or greater, 3.5% (w/w) or greater, 3.75% (w/w %) or grater, 4% (w/w) or greater, 4.25% (w/w) or greater 4.5% (w/w) or greater, 4.75% (w/w) or greater, 5% (w/w) or greater, 5.25% (w/w) or greater, 5.5% (w/w) or greater, 5.75% (w/w) or greater, 6% (w/w) or greater, 6.25% (w/w) or greater, 6.5% (w/w) or greater, 6.75% (w/w) or greater, 7% (w/w) or greater, 7.25% (w/w) or greater, 7.5% (w/w) or greater, 7.75% (w/w) or greater, 8% (w/w) or greater, 8.25% (w/w) or greater, 8.5% (w/w) or greater, 8.75% (w/w) or greater, 9% (w/w) or greater, 9.25% (w/w) or greater, 9.5% (w/w) or greater, 9.75% (w/w) or greater, 10% (w/w) or greater, 10.25% (w/w) or greater, 10.5% (w/w) or greater, 10.75% (w/w) or greater, 11% (w/w) or greater, 11.25% (w/w) or greater, 11.5% (w/w) or greater, 11.75% (w/w) or greater, 12% (w/w) or greater, 12.25% (w/w) or greater, 12.5% (w/w) or greater, 12.75% (w/w) or greater, 13% (w/w) or greater, 13.25% (w/w) or greater, 13.5% (w/w) or greater, 13.75% (w/w) or greater, 14% (w/w) or greater, 14.25% (w/w) or greater, 14.5% (w/w) or greater, 14.75% (w/w) or greater, 15% (w/w) or greater, 15.25% (w/w) or greater, 15.5% (w/w) or greater, 15.75% (w/w) or greater, 16% (w/w) or greater, 16.25% (w/w) or greater, 16.5% (w/w) or greater, 16.75% (w/w) or greater, 17% (w/w) or greater, 17.25% (w/w) or greater, 17.5% (w/w) or greater, 17.75% (w/w) or greater, 18% (w/w) or greater, 18.25% (w/w) or greater, 18.5% (w/w) or greater, 18.75% (w/w) or greater, 19% (w/w) or greater, 19.25% (w/w) or greater, 19.5% (w/w) or greater, 19.75% (w/w) or greater, or 20% (w/w) or greater.

In certain embodiments, the composition comprises compound 1 in an amount from 0.1% (w/w) to 20% (w/w), such as, for example, from 0.1% (w/w) to 15% (w/w), from 0.1% (w/w) to 10% (w/w), from 0.1% (w/w) to 5% (w/w), from 0.1% (w/w) to 2.5% (w/w), from 0.1% (w/w) to 1% (w/w), from 0.25% (w/w) to 20% (w/w), from 0.25% (w/w) to 15% (w/w), from 0.25% (w/w) to 10% (w/w), from 0.25% (w/w) to 5%, from 0.25% to 2.5 (w/w), from 0.5% (w/w) to 20% (w/w), from 0.5% (w/w) to 15% (w/w), from 0.5% (w/w) to 10% (w/w), from 0.5% (w/w) to 5% (w/w), from 0.5% (w/w) to 2.5% (w/w), from 0.5% (w/w) to 1% (w/w), from 10% (w/w) to 20% (w/w), from 10% (w/w) to 150% (w/w), from 10% (w/w) to 10% (w/w), from 1% (w/w) to 5% (w/w), from 1% (w/w) to 2.5% (w/w), from 2.5% (w/w) to 20% (w/w), from 2.5% (w/w) to 15% (w/w), from 2.5% (w/w) to 10% (w/w), from 2.5% (w/w) to 5% (w/w), from 5% (w/w) to 20% (w/w), from 5% (w/w) to 15% (w/w), from 5% (w/w) to 10% (w/w), from 10% (w/w) to 20% (w/w), from 10% (w/w) to 15% (w/w), or from 15% (w/w) to 20% (w/w).

In certain embodiments, the composition comprises compound 1 in an amount from 3% (w/w) to 20% (w/w), such as, for example, from 3% (w/w) to 15% (w/w), from 3% (w/w) to 12% (w/w), from 3% (w/w) to 10% (w/w), from 3% (w/w) to 9% (w/w), or from 3% (w/w) to 6% (w/w).

In certain embodiments, the composition comprises compound 1 in an amount from 6% (w/w) to 20% (w/w), such as, for example, from 6% (w/w) to 15% (w/w), from 6% (w/w) to 12% (w/w), or from 6% (w/w) to 10% (w/w). In certain embodiments, the composition comprises compound 1 in an amount of about 9% (w/w).

In certain embodiments, compound 1 is present as polymorph B. Polymorph B is described as “polymorph 1” in U.S. Pat. No. 9,056,833, the contents of which are incorporated by reference in its entirety. Polymorph B can be characterized by one or more of the following:
(a) characteristic reflections in an X-ray powder diffractogram using CuKα1 radiation in transmission mode at a 2θ angle [° ] of :41b 6.7±0.2, 13.2±0.2, 17.6±0.2, 19.1±0.2, 20.0±0.2, 21.4±0.2, and 22.5±0.2;
(b) characteristic signals in an FT (Fourier-Transformation) Raman spectrum using a near infrared laser (λ=1064 nm) at 3050±2 cm⁻¹, 2929±2 cm⁻¹, 2887±2 cm⁻¹, 1605±2 cm⁻¹, 1293±2 cm⁻¹, and 1042±2 cm⁻¹;
(c) melting point with a DSC onset temperature of 115.5±1° C.;
(d) crystal parameters determine by single crystal structural analysis, e.g., monoclinic space group P2₁/c with one molecule in the asymmetric unit (z=4, a=11.31±0.01 Å, b=8.44±0.01 Å, c=26.86±0.01 Å, β=101.80±0.01 Å, cell volume (V)=2510.0 Å³, ρ=1.269 Mgm⁻³ at room temperature).

In certain embodiments, compound 1 is provided as substantially pure polymorph B, i.e., at least 95% polymorph B (the remainder being other polymorphic or amorphous forms), at least 96% polymorph B, at least 97% polymorph B, at least 98% polymorph B, or at least 99% polymorph B.

The compositions described herein also comprise one or more mucoadhesive polymers. Without wishing to be bound by theory, it is believed that the mucoadhesive polymer may provide for adherence of the composition to mucosal tissue and fluid resulting in improved prevention or treatment of sleep disorders, e.g., OSA.

Exemplary mucoadhesive polymers include, e.g., polyacrylic acid derivatives, cellulose derivatives, natural polymers, polyvinyl pyrrolidone (PVP) polymers, dextran polymers, polyethylene oxide (PEG) polymers, thermoreversible polymers, ionic responsive polymers, copolymer of polymethyl vinyl ether and maleic anhydride, polyvinyl alcohol polymers, salts of the foregoing, derivatives of the foregoing, and combinations thereof.

In certain embodiments, the mucoadhesive polymer comprises or consists of polyacrylic acid, salts (e.g., sodium salts) thereof, and/or derivatives thereof. Exemplary polyacrylic acid mucoadhesive polymers include, e.g., polyacrylic acid, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), carbomer, salts of the forgoing, derivatives of the forgoing, and combinations thereof.

Exemplary carbomers include, but are not limited to, carbomer homopolymer type A (allyl pentaerythritol crosslinked)), carbomer homopolymer type B (allyl pentaerythritol crosslinked, allyl sucrose crosslinked), carbomer homopolymer type C (allyl pentaerythritol crosslinked), carbomer copolymer type B (allyl pentaerythritol crosslinked), and carbomer copolymer type C (allyl pentaerythritol crosslinked).

In certain embodiments, the mucoadhesive polymer comprises or consists of a cellulose derivative. Exemplary cellulose derivatives include, e.g., hydroxy alkyl cellulose polymers, methyl cellulose polymers, carboxymethyl cellulose (CMC) polymers, salts of carboxymethyl cellulose, and combinations thereof. In certain embodiments, the mucoadhesive polymer comprises a hydroxy alkyl cellulose polymer. In certain embodiments, the mucoadhesive polymer comprises hydroxypropyl methylcellulose (HPMC). In certain embodiments, the mucoadhesive polymer comprises hydroxypropyl cellulose (HPC). In certain embodiments, the mucoadhesive polymer comprises a hydroxyethyl cellulose polymer. In certain embodiments, the mucoadhesive polymer comprises a methyl cellulose polymer.

In certain embodiments, the mucoadhesive polymer comprises or consists of a natural polymer. Exemplary natural polymers include, e.g., arabic gum, tragacanth gum, agar polymer, xanthan gum, guar gum, copolymer of alginic acid and sodium alginate, chitosan polymer, pectin, carrageenan, pullulan polymer, modified starch, gelatin, salts of the foregoing, derivatives of the foregoing, and combinations thereof.

In certain embodiments, the mucoadhesive polymer comprises or consists of a polyvinyl pyrrolidone (PVP) polymer, e.g., povidones, copovidones, and crospovidones.

In certain embodiments, the mucoadhesive polymer comprises carrageenan. In certain embodiments, the carrageenan is selected from iota carrageenan, kappa carrageenan, lambda carrageenan, and a combination thereof. In certain embodiments, the carrageenan comprises lambda carrageenan. In certain embodiments, the carrageenan is lambda carrageenan. In certain embodiments, the carrageenan is a non-gelling carrageenan. In certain embodiments, the carrageenan is Viscarin GP 109 or Viscarin GP 209NF.

In certain embodiments, the mucoadhesive polymer comprises or consists of a thermoreversible polymer. Exemplary thermoreversible polymers include, e.g., poloxamers including polyethylene-polypropylene glycols. Non-limiting examples include those commercially available as LUTROL F-127 (alpha-hydro-omega-hydroxypoly(oxyethylene)a poly(oxypropy-lene)b poly(oxyethylene)a block copolymer or polyethylene-polypropylene glycol) (polaxamer 407) and LUTROL F-68 (polaxamer 188), LUTROL F-108 (polaxamer 338) (commercially available from BASF), ethylhydroxy ethylcellulose (EHEC), and combinations thereof.

In certain embodiments, the mucoadhesive polymer comprises or consists of copolymers of polymethyl vinyl ether and maleic anhydride, e.g., copolymers commercially available under the GANTREZ tradename including GANTREZ S and GANTREZ MS type copolymers.

In certain embodiments, the mucoadhesive polymer comprises or consists of PEG 3350, polycarbophil, polyquaternium-10, polyquatemium-7, Sepineo P600, Tyloxapol, or a combination thereof.

In certain embodiments, the composition comprises mucoadhesive polymer in an amount from 0.1% (w/w) to 20% (w/w), such as, for example, from 0.1% (w/w) to 15% (w/w), from 0.1% (w/w) to 10% (w/w), from 0.1% (w/w) to 5% (w/w), from 0.1% (w/w) to 2.5% (w/w), from 0.10% (w/w) to 1% (w/w), from 1% (w/w) to 20% (w/w), from 1% (w/w) to 150% (w/w), from 1% (w/w) to 10% (w/w), from 1% (w/w) to 5% (w/w), from 1% (w/w) to 2.5% (w/w), from 2.5% (w/w) to 20% (w/w), from 2.5% (w/w) to 15% (w/w), from 2.5% (w/w) to 10% (w/w), from 2.5% (w/w) to 5% (w/w), from 5% (w/w) to 20% (w/w), from 5% (w/w) to 15% (w/w), from 5% (w/w) to 10% (w/w), from 10% (w/w) to 20% (w/w), from 10% (w/w) to 15% (w/w), or from 15% (w/w) to 20% (w/w).

In certain embodiments, the composition comprises mucoadhesive polymer in an amount from 0.1% (w/w) to 1% (w/w), such as, for example, from 0.1% (w/w) to 0.6% (w/w), from 0.10% (w/w) to 0.50% (w/w), from 0.10% (w/w) to 0.4% (w/w), from 0.10% (w/w) to 0.30% (w/w), from 0.3% (w/w) to 1% (w/w), or from 0.3% (w/w) to 0.6% (w/w).

In certain embodiments, the composition does not comprise microcrystalline cellulose. In certain embodiments, the composition is free of microcrystalline cellulose.

In certain embodiments, the composition further comprises one or more tonicity modifiers, wetting agents, and/or preservatives.

In certain embodiments, the composition further comprises one or more tonicity modifiers. Exemplary tonicity modifiers include, e.g., dextrose, lactose, sodium chloride, calcium chloride, magnesium chloride, potassium chloride, sorbitol, sucrose, mannitol, trehalose, raffinose, polyethylene glycol, hydroxyethyl starch, glycine, glycerin, sodium acetate, sodium sulfate, and combinations thereof. In certain embodiments, the tonicity modifier comprises sorbitol. In certain embodiments, the tonicity modifier comprises mannitol.

In certain embodiments, the composition comprises one or more tonicity modifiers in an amount from 0.1% (w/w) to 10% (w/w), such as, for example, from 0.1% (w/w) to 5% (w/w), from 0.1% (w/w) to 2.5% (w/w), from 0.1% (w/w) to 1% (w/w), from 1% (w/w) to 10% (w/w), from 1% (w/w) to 5% (w/w), from 1% (w/w) to 2.5% (w/w), from 2.5% (w/w) to 10% (w/w), from 2.5% (w/w) to 5% (w/w), or from 5% (w/w) to 10% (w/w). In certain embodiments, the composition comprises from 0.1% (w/w) to 3% (w/w) tonicity modifier.

In certain embodiments, the composition further comprises one or more wetting agents. Exemplary wetting agents include, e.g., polysorbates, fatty acid glycerol polyethylene glycol esters, fatty acid polyethylene glycol esters, polyethylene glycols, glycerol ethers, cyclodextrins (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), reaction products of castor oil and ethylene oxide (e.g., polyoxyl 35 castor oil or polyoxy 40 hydrogenated castor oil), castor oil, benzalkonium chloride, edetate disodium, N-Dodecyl Beta-D-Maloside, potassium sorbate, sorbitan monolaurate, glycerin, silicon dioxide, magnesium stearate, PEG-8 laurate, poloxamer 125, poloxamer 182, poloxamer 188, poloxamer 407, polypropylene glycol 11 stearyl ether, polypropylene glycol 15 stearyl ether, laureth-4, glyceryl oleate, cetheth-20, sodium monooleate, sodium laureth-3 sulfate, sodium laroyl sarcosinate, sodium lauryl sulfate, tyloxapol, trideceth-10, amonnium lauryl sulfate, ceteareth-12, ceteareth-30, diethanolamine, diisopropanolamine, disodium laureth sulfosuccinate, disodium lauryl sulfosuccinate, docusate sodium, lauramine oxide, laureth sulfate, laureth-23, nonoxynol-9, nonoxynol-40, PEG-60 hydrogenated castor oil, polypropylene glycol, sodium laureth-2 sulfate, sodium laureth-5 sulfate, sodium methyl cocoyl taurate, Steareth-20, Steareth-21, Steareth-40, and combinations thereof.

In certain embodiments, the wetting agent comprises a polysorbate, e.g., polysorbate 20, polysorbate 40, polysorbate 60, or polysorbate 80.

In certain embodiments, the composition comprises one or more wetting agents in an amount from 0.1% (w/w) to 5% (w/w), such as, for example, from 0.1% (w/w) to 4% (w/w), from 0.10% (w/w) to 3% (w/w), from 0.10% (w/w) to 2% (w/w), or from 0.10% (w/w) to 10% (w/w). In certain embodiments, the composition comprises one or more wetting agents in an amount from 0.3% (w/w) to 0.6% (w/w).

In certain embodiments, the composition further comprises one or more preservatives. Exemplary preservatives include, e.g., chlorhexidine gluconate, phenyl ethyl alcohol, 1-phenoxyethanol, benzyl alcohol, sorbic acid, thimerosal, phenylmercuric acetate, benzoates (e.g., sodium benzoate), parabens (e.g., methyl parabens, propyl parabens, and butyl parabens), sorbates, benzalkonium chloride, chlorobutanol, sodium metabisulfate, trisodium citrate dihydrate, boric acid, calcium acetate, dehydroacetic acid, diazolidinyl urea, dichlorobenzyl alcohol, imidurea, methyl salicyclate, methylchloroisothiazolinone, methylchloroisothiazolinone/methylchloroisothiazolinone mixture, propionic acid, sodium sulfite, salts of the foregoing, and combinations thereof. In certain embodiments, the preservative comprises potassium sorbate.

In certain embodiments, the composition comprises one or more preservatives in an amount from 0.01% (w/w) to 2% (w/w), such as, for example, from 0.01% (w/w) to 1.5% (w/w), from 0.01% to 1% (w/w), from 0.01% (w/w) to 0.5% (w/w), from 0.01% (w/w) to 0.25% (w/w), from 0.01% (w/w) to 0.1% (w/w), from 0.01% (w/w) to 0.05% (w/w), or from 0.01% (w/w) to 0.03% (w/w), from 0.03% (w/w) to 2% (w/w), from 0.03% (w/w) to 1.5% (w/w), from 0.03% (w/w) to 1% (w/w), from 0.03% (w/w) to 0.5% (w/w), from 0.03% (w/w) to 0.25% (w/w), from 0.03% (w/w) to 0.1% (w/w), from 0.03% (w/w) to 0.05% (w/w), from 0.05% (w/w) to 2% (w/w), from 0.05% (w/w) to 1.5% (w/w), from 0.05% (w/w) to 1% (w/w), from 0.05% (w/w) to 0.5% (w/w), from 0.05% (w/w) to 0.25% (w/w), from 0.05% (w/w) to 0.1% (w/w), from 0.01% (w/w) to 2% (w/w), from 0.01% (w/w) to 1.5% (w/w), from 0.01% (w/w) to 1% (w/w), from 0.1% (w/w) to 0.5% (w/w), from 0.1% (w/w) to 0.25% (w/w), from 0.25% (w/w) to 2% (w/w), from 0.25% (w/w) to 1.5% (w/w), from 0.25% (w/w) to 1% (w/w), from 0.25% (w/w) to 0.5% (w/w), or from 0.5% (w/w) to 1% (w/w).

In certain embodiments, the composition comprises one or more preservatives in an amount from 0.01% (w/w) to 0.2% (w/w), such as, for example, from 0.05% (w/w) to 0.2% (w/w) or from 0.05% (w/w) to 0.15% (w/w).

The compositions described herein further comprise an aqueous vehicle, i.e., a medium or carrier comprising at least a minimal amount of water, in which the components described hereinabove are dissolved and/or suspended. Exemplary aqueous vehicles include, e.g., deionized water, saline, phosphate buffer, citrate buffer, malate buffer, tartrate buffer, balanced salt solution, salts of organic acids, combinations of organic acids and salts of organic acids (e.g., tribasic sodium citrate and citric acid, malic acid and sodium malate, and potassium sodium tartrate and tartaric acid), acetic acid, hydrochloric acid, potassium phosphate (monobasic), sodium phosphate dibasic (heptahydrate), sodium phosphate monobasic (anhydrous/monohydrate), sodium hydroxide, potassium hydroxide, sodium gluconolactone, lactic acid, nitric acid, sodium acetate, sodium lactate, tromethamine, ammonia, citric acid, calcium acetate, diethanolamine, diisopropanolamine, phosphoric acid, potassium citrate, potassium hydroxide, succinic acid, sulfuric acid, arginine hydrochloride, and combinations thereof. In certain embodiments, the aqueous vehicle comprises citrate buffer. In certain embodiments, the aqueous vehicle comprises phosphate buffer. In certain embodiments, the aqueous vehicle comprises citrate-phosphate buffer comprising citric acid and dibasic sodium phosphate.

In certain embodiments, the composition comprises an aqueous vehicle in an amount from 85% (w/w) to 95% (w/w), such as, for example, from 85% (w/w) to 90% (w/w) or from 90% (w/w) to 95% (w/w).

In certain embodiments, the composition further comprises a chelating agent. Exemplary chelating agents include, but are not limited to, ethylenediaminetetraacetic acid (EDTA), citric acid monohydrate, trisodium HEDTA, edetate calcium disodium, edetate sodium, edetate trisodium, edetic acid, pentasodium pentetate, sodium acetate, and combinations thereof. Without being bound by theory, it is believed that, in certain instances, the presence of a chelating agent (e.g., EDTA) may prevent agglomeration of compound 1 and/or the composition, thereby improving stability of the composition.

In certain embodiments, the composition comprises a chelating agent (e.g., EDTA) in an amount from about 0.01% (w/w) to 2% (w/w), such as, for example, from 0.01% (w/w) to 1.5% (w/w), from 0.010% (w/w) to 1% (w/w), from 0.01% (w/w) to 0.5% (w/w), from 0.01% (w/w) to 0.10% (w/w), from 0.10% (w/w) to 2% (w/w), from 0.10% (w/w) to 1.50% (w/w), from 0.10% (w/w) to 10% (w/w), from 0.10% (w/w) to 0.50% (w/w), from 0.50% (w/w) to 2% (w/w), from 0.50% (w/w) to 1.50% (w/w), from 0.50% (w/w) to 10% (w/w), from 10% (w/w) to 2% (w/w), or from 1.5% (w/w) to 2.0% (w/w).

In certain embodiments, the composition comprises a chelating agent (e.g., EDTA) in an amount from 0.1% (w/w) to 0.5% (w/w), such as, for example, from 0.1% (w/w) to 0.25% (w/w).

### 4.2.1. Formulations

In certain embodiments, a composition comprises compound 1 and a mucoadhesive polymer. In certain embodiments, a composition comprises from 0.1% (w/w) to 20% w/w (such as 6% (w/w) to 20% (w/w) or 6% (w/w) to 12% (w/w)) compound 1 and a mucoadhesive polymer. In certain embodiments, a composition comprises from 0.1% (w/w) to 20% w/w (such as 6% (w/w) to 20% (w/w) or 6% (w/w) to 12% (w/w)) compound 1 and from 0.1% (w/w) to 20% (w/w) of a mucoadhesive polymer.

In certain embodiments, a composition comprises compound 1 and a mucoadhesive polymer, wherein the mucoadhesive polymer is a cellulose derivative (e.g., hydroxypropyl methylcellulose or methyl cellulose). In certain embodiments, a composition comprises from 0.1% (w/w) to 20% w/w (such as 6% (w/w) to 20% (w/w) or 6% (w/w) to 12% (w/w)) compound 1 and a mucoadhesive polymer, wherein the mucoadhesive polymer is a cellulose derivative (e.g., hydroxypropyl methylcellulose or methyl cellulose). In certain embodiments, a composition comprises from 0.1% (w/w) to 20% w/w (such as 6% (w/w) to 20% (w/w) or 6% (w/w) to 12% (w/w)) compound 1 and from 0.1% (w/w) to 20% (w/w) of a mucoadhesive polymer, wherein the mucoadhesive polymer is a cellulose derivative (e.g., hydroxypropyl methylcellulose or methyl cellulose).

In certain embodiments, a composition comprises compound 1 and a mucoadhesive polymer, wherein the mucoadhesive polymer is a natural polymer (e.g., carrageenan). In certain embodiments, a composition comprises from 0.1% (w/w) to 20% w/w (such as 6% (w/w) to 20% (w/w) or 6% (w/w) to 12% (w/w)) compound 1 and a mucoadhesive polymer, wherein the mucoadhesive polymer is a natural polymer (e.g., carrageenan). In certain embodiments, a composition comprises from 0.1% (w/w) to 20% w/w (such as 6% (w/w) to 20% (w/w) or 6% (w/w) to 12% (w/w)) compound 1 and from 0.1% (w/w) to 20% (w/w) of a mucoadhesive polymer, wherein the mucoadhesive polymer is a natural polymer (e.g., carrageenan).

In certain embodiments, a composition comprises compound 1, a mucoadhesive polymer, and a wetting agent. In certain embodiments, a composition comprises from 0.1% (w/w) to 20% w/w (such as 6% (w/w) to 20% (w/w) or 6% (w/w) to 12% (w/w)) compound 1, a mucoadhesive polymer, and a wetting agent. In certain embodiments, a composition comprises from 0.1% (w/w) to 20% w/w (such as 6% (w/w) to 20% (w/w) or 6% (w/w) to 12% (w/w)) compound 1, from 0.1% (w/w) to 20% (w/w) of mucoadhesive polymer, and from 0.1% (w/w) to 50% (w/w) wetting agent.

In certain embodiments, a composition comprises compound 1, a mucoadhesive polymer, and a wetting agent, wherein the mucoadhesive polymer is a cellulose derivative (e.g., hydroxypropyl methylcellulose or methyl cellulose) and the wetting agent is a reaction product of castor oil and ethylene oxide (e.g., polyoxyl 35 castor oil and polyoxyl 40 hydrogenated castor oil). In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a cellulose derivative (e.g., hydroxypropyl methylcellulose or methyl cellulose); and a wetting agent, wherein the wetting agent is a reaction product of castor oil and ethylene oxide (e.g., polyoxyl 35 castor oil and polyoxyl 40 hydrogenated castor oil). In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a cellulose derivative (e.g., hydroxypropyl methylcellulose or methyl cellulose); and from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil).

In certain embodiments, a composition comprises compound 1, a mucoadhesive polymer, and a wetting agent, wherein the mucoadhesive polymer is a natural polymer (e.g., carrageenan) and the wetting agent is a reaction product of castor oil and ethylene oxide (e.g., polyoxyl 35 castor oil and polyoxyl 40 hydrogenated castor oil). In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a natural polymer (e.g., carrageenan); and a wetting agent, wherein the wetting agent is a reaction product of castor oil and ethylene oxide (e.g., polyoxyl 35 castor oil and polyoxyl 40 hydrogenated castor oil). In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a natural polymer (e.g., carrageenan); and from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil).

In certain embodiments, a composition comprises compound 1, a mucoadhesive polymer, a wetting agent, and a tonicity modifier. In certain embodiments, a composition comprises from 0.1% (w/w) to 20% w/w (such as 6% (w/w) to 20% (w/w) or 6% (w/w) to 12% (w/w)) compound 1, a mucoadhesive polymer, a wetting agent, and a tonicity modifier. In certain embodiments, a composition comprises from 0.1% (w/w) to 20% w/w (such as 6% (w/w) to 20% (w/w) or 6% (w/w) to 12% (w/w)) compound 1, from 0.1% (w/w) to 20% (w/w) of mucoadhesive polymer, from 0.1% (w/w) to 5% (w/w) wetting agent, and from 0.1% (w/w) to 10% (w/w) tonicity modifier.

In certain embodiments, a composition comprises compound 1, a mucoadhesive polymer, a wetting agent, and a tonicity modifier, wherein the mucoadhesive polymer is a cellulose derivative (e.g., hydroxypropyl methylcellulose or methyl cellulose), the wetting agent is a reaction product of castor oil and ethylene oxide (e.g., polyoxyl 35 castor oil and polyoxyl 40 hydrogenated castor oil), and the tonicity modifier is selected from sorbitol and mannitol. In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a cellulose derivative (e.g., hydroxypropyl methylcellulose or methyl cellulose); a wetting agent, wherein the wetting agent is a reaction product of castor oil and ethylene oxide (e.g., polyoxyl 35 castor oil and polyoxyl 40 hydrogenated castor oil); and a tonicity modifier is selected from sorbitol and mannitol. In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a cellulose derivative (e.g., hydroxypropyl methylcellulose or methyl cellulose); from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil); and from 0.1% (w/w) to 10% (w/w) tonicity modifier, wherein the tonicity modifier is selected from sorbitol and mannitol.

In certain embodiments, a composition comprises compound 1, a mucoadhesive polymer, a wetting agent, and a tonicity modifier, wherein the mucoadhesive polymer is a natural polymer (e.g., carrageenan), the wetting agent is a reaction product of castor oil and ethylene oxide (e.g., polyoxyl 35 castor oil and polyoxyl 40 hydrogenated castor oil), and the tonicity modifier is sorbitol. In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a natural polymer (e.g., carrageenan); a wetting agent, wherein the wetting agent is a reaction product of castor oil and ethylene oxide (e.g., polyoxyl 35 castor oil and polyoxyl 40 hydrogenated castor oil); and a tonicity modifier, wherein the tonicity modifier is sorbitol. In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a natural polymer (e.g., carrageenan); from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil); and from 0.1% (w/w) to 10% (w/w) tonicity modifier, wherein the tonicity modifier is sorbitol.

In certain embodiments, a composition comprises compound 1, a mucoadhesive polymer, a wetting agent, a tonicity modifier, and a preservative. In certain embodiments, a composition comprises from 0.1% (w/w) to 20% w/w (such as 6% (w/w) to 20% (w/w) or 6% (w/w) to 12% (w/w)) compound 1, a mucoadhesive polymer, a wetting agent, a tonicity modifier, and a preservative. In certain embodiments, a composition comprises from 0.10% (w/w) to 20% w/w (such as 6% (w/w) to 20% (w/w) or 6% (w/w) to 12% (w/w)) compound 1, from 0.1% (w/w) to 20% (w/w) of mucoadhesive polymer, from 0.1% (w/w) to 5% (w/w) wetting agent, from 0.1% (w/w) to 10% (w/w) tonicity modifier, and from 0.01% (w/w) to 2% (w/w) preservative.

In certain embodiments, a composition comprises compound 1, a mucoadhesive polymer, a wetting agent, a tonicity modifier, and a preservative, wherein the mucoadhesive polymer is a cellulose derivative (e.g., hydroxypropyl methylcellulose or methyl cellulose), the wetting agent is a reaction product of castor oil and ethylene oxide (e.g., polyoxyl 35 castor oil and polyoxyl 40 hydrogenated castor oil), and the tonicity modifier is selected from sorbitol and mannitol, and the preservative is potassium sorbate. In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a cellulose derivative (e.g., hydroxypropyl methylcellulose or methyl cellulose); a wetting agent, wherein the wetting agent is a reaction product of castor oil and ethylene oxide (e.g., polyoxyl 35 castor oil and polyoxyl 40 hydrogenated castor oil); a tonicity modifier selected from sorbitol and mannitol; and a preservative, wherein the preservative is potassium sorbate. In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a cellulose derivative (e.g., hydroxypropyl methylcellulose or methyl cellulose); from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil); from 0.1% (w/w) to 10% (w/w) tonicity modifier, wherein the tonicity modifier is selected from sorbitol and mannitol; and from 0.01% (w/w) to 2% (w/w) preservative, wherein the preservative is potassium sorbate.

In certain embodiments, a composition comprises compound 1, a mucoadhesive polymer, a wetting agent, a tonicity modifier, and a preservative, wherein the mucoadhesive polymer is a natural polymer (e.g., carrageenan), the wetting agent is a reaction product of castor oil and ethylene oxide (e.g., polyoxyl 35 castor oil and polyoxyl 40 hydrogenated castor oil), the tonicity modifier is sorbitol, and the preservative is potassium sorbate. In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a natural polymer (e.g., carrageenan); a wetting agent, wherein the wetting agent is a reaction product of castor oil and ethylene oxide (e.g., polyoxyl 35 castor oil and polyoxyl 40 hydrogenated castor oil); a tonicity modifier, wherein the tonicity modifier is sorbitol; and a preservative, wherein the preservative is potassium sorbate. In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a natural polymer (e.g., carrageenan); from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil); from 0.1% (w/w) to 10% (w/w) tonicity modifier, wherein the tonicity modifier is sorbitol; and from 0.01% (w/w) to 2% (w/w) preservative, wherein the preservative is potassium sorbate.

In certain embodiments, a composition comprises compound 1, a mucoadhesive polymer, a wetting agent, a tonicity modifier, a preservative, and a chelating agent. In certain embodiments, a composition comprises from 0.1% (w/w) to 20% w/w (such as 6% (w/w) to 20% (w/w) or 6% (w/w) to 12% (w/w)) compound 1, a mucoadhesive polymer, a wetting agent, a tonicity modifier, a preservative, and a chelating agent. In certain embodiments, a composition comprises from 0.1% (w/w) to 20% w/w (such as 6% (w/w) to 20% (w/w) or 6% (w/w) to 12% (w/w)) compound 1, from 0.1% (w/w) to 20% (w/w) of mucoadhesive polymer, from 0.1% (w/w) to 5% (w/w) wetting agent, from 0.1% (w/w) to 10% (w/w) tonicity modifier, from 0.01% (w/w) to 2% (w/w) preservative, and from 0.01% (w/w) to 2% (w/w) chelating agent.

In certain embodiments, a composition comprises compound 1, a mucoadhesive polymer, a wetting agent, a tonicity modifier, a preservative, and a chelating agent, wherein the mucoadhesive polymer is a natural polymer (e.g., carrageenan), the wetting agent is a reaction product of castor oil and ethylene oxide (e.g., polyoxyl 35 castor oil and polyoxyl 40 hydrogenated castor oil), the tonicity modifier is sorbitol, the preservative is potassium sorbate, and the chelating agent is EDTA. In certain embodiments, a composition comprises from 0.1% (w/w) to 20% w/w (such as 6% (w/w) to 20% (w/w) or 6% (w/w) to 12% (w/w)) compound 1; a mucoadhesive polymer, wherein the mucoadhesive polymer is a natural polymer (e.g., carrageenan); a wetting agent, wherein the wetting agent is a reaction product of castor oil and ethylene oxide (e.g., polyoxyl 35 castor oil and polyoxyl 40 hydrogenated castor oil); a tonicity modifier, wherein the tonicity modifier is sorbitol; a preservative, wherein the preservative is potassium sorbate; and a chelating agent, wherein the chelating agent is EDTA. In certain embodiments, a composition comprises from 0.1% (w/w) to 20% w/w (such as 6% (w/w) to 20% (w/w) or 6% (w/w) to 12% (w/w)) compound 1; from 0.1% (w/w) to 20% (w/w) of a mucoadhesive polymer, wherein the mucoadhesive polymer is a natural polymer (e.g., carrageenan); from 0.1% (w/w) to 5% (w/w) wetting agent, wherein the wetting agent is a reaction product of castor oil and ethylene oxide (e.g., polyoxyl 35 castor oil and polyoxyl 40 hydrogenated castor oil); from 0.1% (w/w) to 10% (w/w) tonicity modifier, wherein the tonicity modifier is sorbitol; from 0.01% (w/w) to 2% (w/w) preservative, wherein the preservative is potassium sorbate; and from 0.01% (w/w) to 2% (w/w) chelating agent, wherein the chelating agent is EDTA.

In certain embodiments, a composition comprises:
a. from 0.1% (w/w) to 20% (w/w) compound 1, such as 6% (w/w) to 20% (w/w) or 6% (w/w) to 12% (w/w);
b. from 0.1% (w/w) to 20% (w/w) mucoadhesive polymer;
c. from 0.1% (w/w) to 10% (w/w) tonicity modifier;
d. from 0.1% (w/w) to 5% (w/w) wetting agent;
e. from 0.01% (w/w) to 2% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 0.1% (w/w) to 20% (w/w) compound 1, such as 6% (w/w) to 20% (w/w) or 6% (w/w) to 12% (w/w);
b. from 0.1% (w/w) to 20% (w/w) mucoadhesive polymer comprising methyl cellulose, HMPC, carrageenan, or a combination thereof;
c. from 0.1% (w/w) to 10% (w/w) tonicity modifier comprising mannitol, sorbitol, or a combination thereof;
d. from 0.1% (w/w) to 5% (w/w) wetting agent comprising polyoxyl 40 hydrogenated castor oil, polyoxyl 35 castor oil, or a combination thereof;
e. from 0.01% (w/w) to 2% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 0.1% (w/w) to 20% (w/w) compound 1, such as 6% (w/w) to 20% (w/w) or 6% (w/w) to 12% (w/w);
b. from 0.1% (w/w) to 1% (w/w) mucoadhesive polymer;
c. from 0.1% (w/w) to 5% (w/w) tonicity modifier;
d. from 0.1% (w/w) to 1% (w/w) wetting agent;
e. from 0.01% (w/w) to 2% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 0.1% (w/w) to 20% (w/w) compound 1, such as 6% (w/w) to 20% (w/w) or 6% (w/w) to 12% (w/w);
b. from 0.1% (w/w) to 1% (w/w) mucoadhesive polymer comprising methyl cellulose, HMPC, carrageenan, or a combination thereof;
c. from 0.1% (w/w) to 5% (w/w) tonicity modifier comprising mannitol, sorbitol, or a combination thereof;
d. from 0.1% (w/w) to 1% (w/w) wetting agent comprising polyoxyl 40 hydrogenated castor oil, polyoxyl 35 castor oil, or a combination thereof;
e. from 0.01% (w/w) to 2% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 0.1% (w/w) to 20% (w/w) compound 1, such as 6% (w/w) to 20% (w/w) or 6% (w/w) to 12% (w/w);
b. from 0.3% (w/w) to 0.6% (w/w) mucoadhesive polymer;
c. from 0.1% (w/w) to 3% (w/w) tonicity modifier;
d. from 0.3% (w/w) to 0.6% (w/w) wetting agent;
e. from 0.01% (w/w) to 0.15% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 0.1% (w/w) to 20% (w/w) compound 1, such as 6% (w/w) to 20% (w/w) or 6% (w/w) to 12% (w/w);
b. from 0.3% (w/w) to 0.6% (w/w) mucoadhesive polymer comprising methyl cellulose, HMPC, carrageenan, or a combination thereof;
c. from 0.1% (w/w) to 3% (w/w) tonicity modifier comprising mannitol, sorbitol, or a combination thereof;
d. from 0.3% (w/w) to 0.6% (w/w) wetting agent comprising polyoxyl 40 hydrogenated castor oil, polyoxyl 35 castor oil, or a combination thereof;
e. from 0.01% (w/w) to 0.15% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 3% (w/w) to 20% (w/w) compound 1, such as 6% (w/w) to 20% (w/w) or 6% (w/w) to 12% (w/w);
b. from 0.1% (w/w) to 20% (w/w) mucoadhesive polymer;
c. from 0.1% (w/w) to 10% (w/w) tonicity modifier;
d. from 0.1% (w/w) to 5% (w/w) wetting agent;
e. from 0.01% (w/w) to 2% (w/w) preservative;
f. from 85% (w/w) to 95% (w/w) aqueous vehicle, and
g. optionally, from 0.01% (w/w) to 2% (w/w) chelating agent (e.g., EDTA).

In certain embodiments, a composition comprises:
a. from 3% (w/w) to 20% (w/w) compound 1, such as 6% (w/w) to 20% (w/w) or 6% (w/w) to 12% (w/w);
b. from 0.1% (w/w) to 20% (w/w) mucoadhesive polymer comprising methyl cellulose, HMPC, carrageenan, or a combination thereof;
c. from 0.1% (w/w) to 10% (w/w) tonicity modifier comprising mannitol, sorbitol, or a combination thereof;
d. from 0.1% (w/w) to 5% (w/w) wetting agent comprising polyoxyl 40 hydrogenated castor oil, polyoxyl 35 castor oil, or a combination thereof;
e. from 0.01% (w/w) to 2% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 3% (w/w) to 20% (w/w) compound 1, such as 6% (w/w) to 20% (w/w) or 6% (w/w) to 12% (w/w);
b. from 0.1% (w/w) to 1% (w/w) mucoadhesive polymer;
c. from 0.1% (w/w) to 5% (w/w) tonicity modifier;
d. from 0.10% (w/w) to 10% (w/w) wetting agent;
e. from 0.01% (w/w) to 2% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 3% (w/w) to 20% (w/w) compound 1, such as 6% (w/w) to 20% (w/w) or 6% (w/w) to 12% (w/w);
b. from 0.1% (w/w) to 1% (w/w) mucoadhesive polymer comprising methyl cellulose, HMPC, carrageenan, or a combination thereof;
c. from 0.1% (w/w) to 5% (w/w) tonicity modifier comprising mannitol, sorbitol, or a combination thereof;
d. from 0.1% (w/w) to 1% (w/w) wetting agent comprising polyoxyl 40 hydrogenated castor oil, polyoxyl 35 castor oil, or a combination thereof;
e. from 0.01% (w/w) to 2% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 3% (w/w) to 20% (w/w) compound 1, such as 6% (w/w) to 20% (w/w) or 6% (w/w) to 12% (w/w);
b. from 0.3% (w/w) to 0.6% (w/w) mucoadhesive polymer;
c. from 0.1% (w/w) to 3% (w/w) tonicity modifier;
d. from 0.3% (w/w) to 0.6% (w/w) wetting agent;
e. from 0.01% (w/w) to 0.15% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 3% (w/w) to 20% (w/w) compound 1, such as 6% (w/w) to 20% (w/w) or 6% (w/w) to 12% (w/w);
b. from 0.3% (w/w) to 0.6% (w/w) mucoadhesive polymer comprising methyl cellulose, HMPC, carrageenan, or a combination thereof;
c. from 0.1% (w/w) to 3% (w/w) tonicity modifier comprising mannitol, sorbitol, or a combination thereof;
d. from 0.3% (w/w) to 0.6% (w/w) wetting agent comprising polyoxyl 40 hydrogenated castor oil, polyoxyl 35 castor oil, or a combination thereof;
e. from 0.01% (w/w) to 0.15% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 3% (w/w) to 10% (w/w) compound 1, such as 6% (w/w) to 20% (w/w) or 6% (w/w) to 12% (w/w);
b. from 0.1% (w/w) to 20% (w/w) mucoadhesive polymer;
c. from 0.1% (w/w) to 10% (w/w) tonicity modifier;
d. from 0.1% (w/w) to 5% (w/w) wetting agent;
e. from 0.01% (w/w) to 1% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 3% (w/w) to 10% (w/w) compound 1, such as 6% (w/w) to 20% (w/w) or 6% (w/w) to 12% (w/w);
b. from 0.1% (w/w) to 20% (w/w) mucoadhesive polymer comprising methyl cellulose, HMPC, carrageenan, or a combination thereof;
c. from 0.1% (w/w) to 10% (w/w) tonicity modifier comprising mannitol, sorbitol, or a combination thereof;
d. from 0.1% (w/w) to 5% (w/w) wetting agent comprising polyoxyl 40 hydrogenated castor oil, polyoxyl 35 castor oil, or a combination thereof;
e. from 0.01% (w/w) to 2% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 3% (w/w) to 10% (w/w) compound 1, such as 6% (w/w) to 20% (w/w) or 6% (w/w) to 12% (w/w);
b. from 0.1% (w/w) to 1% (w/w) mucoadhesive polymer;
c. from 0.1% (w/w) to 5% (w/w) tonicity modifier;
d. from 0.10% (w/w) to 10% (w/w) wetting agent;
e. from 0.01% (w/w) to 2% (w/w) preservative,
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 3% (w/w) to 10% (w/w) compound 1, such as 6% (w/w) to 20% (w/w) or 6% (w/w) to 12% (w/w);
b. from 0.1% (w/w) to 1% (w/w) mucoadhesive polymer comprising methyl cellulose, HMPC, carrageenan, or a combination thereof;
c. from 0.1% (w/w) to 5% (w/w) tonicity modifier comprising mannitol, sorbitol, or a combination thereof;
d. from 0.1% (w/w) to 1% (w/w) wetting agent comprising polyoxyl 40 hydrogenated castor oil, polyoxyl 35 castor oil, or a combination thereof;
e. from 0.01% (w/w) to 2% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

In certain embodiments, a composition comprises:
a. from 3% (w/w) to 10% (w/w) compound 1, such as 6% (w/w) to 20% (w/w) or 6% (w/w) to 12% (w/w);
b. from 0.3% (w/w) to 0.6% (w/w) mucoadhesive polymer;
c. from 0.1% (w/w) to 3% (w/w) tonicity modifier;
d. from 0.3% (w/w) to 0.6% (w/w) wetting agent;
e. from 0.01% (w/w) to 0.15% (w/w) preservative;
f. from 85% (w/w) to 95% (w/w) aqueous vehicle; and
g. optionally, from 0.01% (w/w) to 2% (w/w) chelating agent (e.g., EDTA).

In certain embodiments, a composition comprises:
a. from 3% (w/w) to 10% (w/w) compound 1, such as 6% (w/w) to 20% (w/w) or 6% (w/w) to 12% (w/w);
b. from 0.3% (w/w) to 0.6% (w/w) mucoadhesive polymer comprising methyl cellulose, HMPC, carrageenan, or a combination thereof;
c. from 0.1% (w/w) to 3% (w/w) tonicity modifier comprising mannitol, sorbitol, or a combination thereof;
d. from 0.3% (w/w) to 0.6% (w/w) wetting agent comprising polyoxyl 40 hydrogenated castor oil, polyoxyl 35 castor oil, or a combination thereof;
e. from 0.01% (w/w) to 0.15% (w/w) preservative comprising potassium sorbate;
f. from 85% (w/w) to 95% (w/w) aqueous vehicle comprising citrate buffer, phosphate buffer, or a combination thereof; and
g. optionally, from 0.01% (w/w) to 2% (w/w) chelating agent (e.g., EDTA).

### 4.2.2. Properties

In certain embodiments, a pharmaceutical microparticulate suspension composition comprises less than 0.1% (w/w) of compound 1 in solution, e.g., less than 0.05% (w/w), or less than 0.025% (w/w).

In certain embodiments, a pharmaceutical microparticulate suspension composition comprises from 0.001% (w/w) to 0.1 (w/w) compound 1 in solution, e.g., from 0.001% (w/w) to 0.05% (w/w), from 0.005% (w/w/) to 0.05% (w/w), or from 0.005% (w/w) to 0.025% (w/w).

In certain embodiments, the pharmaceutical composition has an osmolality from 200 mOsm/kg to 500 mOsm/kg, such as, for example, from 200 mOsm/kg to 450 mOsm/kg, from 200 mOsm/kg to 400 mOsm/kg, from 200 mOsm/kg to 350 mOsm/kg, from 200 mOsm/kg to 300 mOsm/kg, from 200 mOsm/kg to 250 mOsm/kg, from 250 mOsm/kg to 500 mOsm/kg, from 250 mOsm/kg to 450 mOsm/kg, from 250 mOsm/kg to 400 mOsm/kg, from 250 mOsm/kg to 350 mOsm/kg, from 250 mOsm/kg to 300 mOsm/kg, from 300 mOsm/kg to 500 mOsm/kg, from 300 mOsm/kg to 450 mOsm/kg, from 300 mOsm/kg to 400 mOsm/kg, from 300 mOsm/kg to 350 mOsm/kg, from 350 mOsm/kg to 500 mOsm/kg, from 350 mOsm/kg to 450 mOsm/kg, from 350 mOsm/kg to 400 mOsm/kg, from 400 mOsm/kg to 500 mOsm/kg, from 400 mOsm/kg to 450 mOsm/kg, or from 450 mOsm/kg to 500 mOsm/kg. In certain embodiments, the pharmaceutical composition has an osmolality from 250 mOsm/kg to 350 mOsm/kg. mOsm/kg means milli-Osmole per kilogram. One milliosmole is one milli-mole (i.e. 1 thousandth of a mole) of an osmotically active compound, i.e. compounds that contribute to the osmotic pressure of a solution.

In certain embodiments, the pharmaceutical composition has an apparent pH from 5 to 7, such as, for example, from 5 to 6.5, from 5 to 6, from 5 to 5.5, from 6 to 7, from 6 to 6.5, or from 6.5 to 7. In certain embodiments, the apparent pH of the pharmaceutical composition is from 5.5 to 6.5 or from 6 to 6.5.

In certain embodiments, the particle size distribution (D₁₀) of compound 1 in the pharmaceutical composition is from 0.1 μm to 5 μm, such as, for example, from 0.1 μm to 4 μm, from 0.1 μm to 3 μm, from 0.1 μm to 2 μm, from 0.1 μm to 1 μm, from 0.5 μm to 5 μm, from 0.5 μm to 4 μm, from 0.5 μm to 3 μm, from 0.5 μm to 2 μm, from 0.5 μm to 1 μm, from 1 μm to 5 μm, from 1 μm to 4 μm, from 1 μm to 3 μm, from 1 μm to 2 μm, from 2 μm to 5 μm, from 3 μm to 5 μm, or from 4 μm to 5. In certain embodiments, the particle size distribution (D₁₀) of compound 1 in the pharmaceutical composition is from 0.1 μm to 1.5 μm. In certain embodiments, the particle size distribution (D₁₀) of compound 1 in the pharmaceutical composition is from 0.1 μm to 3 μm.

In certain embodiments, the particle size distribution (D₅₀) of compound 1 in the pharmaceutical composition is from 2 μm to 15 μm, such as, for example, from 2 μm to 10 μm, from 2 μm to 5 μm, from 5 μm to 15 μm, from 5 μm to 10 μm, or from 10 μm to 15 μm. In certain embodiments, the particle size distribution (D₅₀) of compound 1 in the pharmaceutical composition is from 2 μm to 5 μm. In certain embodiments, the particle size distribution (D₅₀) of compound 1 in the pharmaceutical composition is from 5 μm to 15 μm.

In certain embodiments, the particle size distribution (D₉₀) of compound 1 in the pharmaceutical composition is from 4 μm to 50 μm, such as, for example, from 4 μm to 45 μm, from 4 μm to 40 μm, from 4 μm to 35 μm, from 4 μm to 30 μm, from 4 μm to 25 μm, from 4 to 20 μm, from 4 μm to 10 μm, from 5 μm to 45 μm, from 5 μm to 40 μm, from 5 μm to 35 μm, from 5 μm to 30 μm, from 5 μm to 25 μm, from 5 to 20 μm, from 5 μm to 10 μm, from 10 μm to 50 μm, from 10 μm to 45 μm, from 10 μm to 40 μm, from 10 μm to 35 μm, from 10 μm to 30 μm, from 10 μm to 20 μm, from 20 μm to 50 μm, from 20 μm to 45 μm, from 20 μm to 40 μm, from 20 μm to 35 μm, 20 μm to 30 μm, from 20 μm to 25 μm, from 25 μm to 50 μm, from 25 μm to 45 μm, from 25 μm to 40 μm, from 25 μm to 35 μm, from 25 μm to 30 μm, from 30 μm to 50 μm, from 30 μm to 45 μm, from 30 μm to 40 μm, from 30 μm to 35 μm, from 35 μm to 50 μm, from 35 μm to 45 μm, from 35 μm to 40 μm, from 40 μm to 50 μm, or from 45 μm to 50 μm. In certain embodiments, the particle size distribution (D₉₀) of compound 1 in the pharmaceutical composition is from 4 μm to 15 μm. In certain embodiments, the particle size distribution (D₉₀) of compound 1 in the pharmaceutical composition is from 10 μm to 35 μm.

In certain embodiments, compound 1 has a D₁₀<3 μm, D₅₀<15 μm, and D₉₀<50 μm. In certain embodiments, compound 1 has a D₁₀<3 μm, D₅₀<15 μm, and D₉₀<35 μm.

In certain embodiments, compound 1 has a D₁₀<2 μm, D₅₀<5 μm, and D₉₀<10 μm. In certain embodiments, compound 1 has a D₁₀ from 0.1 to 1.5 μm, D₅₀ from 2 to 5 μm, and D₉₀ from 5 to 10 μm.

In certain embodiments, compound 1 has a D₁₀ from 0.1 to 3 μm, D₅₀ from 5 to 15 μm, and D₉₀ from 10 to 50 μm. In certain embodiments, compound 1 has a D₁₀ from 0.1 to 3 μm, D₅₀ from 5 to 15 μm, and D₉₀ from 10 to 35 μm.

In certain embodiments, the particle size distribution (D₉₀) of compound 1 in the pharmaceutical composition is bimodal. In certain embodiments, 25% (w/w) to 75% (w/w) of compound 1 has a D₉₀ from 20 μm to 30 μm and the remainder of compound 1 has a D₉₀ from 5 μm to 15 μm.

In certain embodiments, the pharmaceutical composition has a viscosity from 1 cP to 100 cP, such as, for example, from 20 cP to 100 cP, from 20 cP to 50 cP, from 20 cP to 40 cP, from 20 cP to 30 cP, from 25 cP to 50 cP, from 25 cP to 40 cP, from 25 Cp to 30, from 30 cP to 50 cP, from 30 cP to 40 cP, or from 40 cP to 50 cP.

In certain embodiments, the pharmaceutical composition has a shear-thinning rheology. In certain other embodiments, the pharmaceutical composition has a shear-thickening rheology. In still other embodiments, the pharmaceutical composition has a Newtonian rheology.

In certain embodiments, the pharmaceutical composition has storage dominant behavior (i.e., is more elastic/solid) at low torque in oscillation amplitude experiments. In certain embodiments, the pharmaceutical composition has loss dominant behavior (i.e., is more viscous/liquid) at low torque in oscillation amplitude experiments.

In certain embodiments, the pharmaceutical composition has a surface tension from 40 to 50 mN/m, e.g., from 40 to 45 mN/m or from 45 to 50 mN/m

In certain embodiments, the pharmaceutical composition is stable when stored at 25° C. and/or 40° C. for at least 2 weeks (“storage conditions”) as determined by one or more of the following: (i) compound 1 recovery, (ii) macroscopic appearance, (iii) microscopic appearance, (iv) apparent pH, (v) osmolality, (vi) particle size distribution (D₉₀), (vii) droplet size, and (viii) Raman analysis. In certain embodiments, the pharmaceutical composition is stable when stored at 25° C. and/or 40° C. for at least 4 weeks, 8 weeks, 4 months, 5 months, 6 months, 7 months, eight months, nine months, ten months, eleven months, or twelve months or more. “Stable” is further characterized for each of (i)-(viii) in the sections below.

Compound 1 recovery can be determined by, e.g., comparing compound 1 content (e.g., by HPLC) in the pharmaceutical composition at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, compound 1 recovery at the later time point is at least 90% of the compound 1 content at t=0, such as, for example, at least 91%, at least 92%, at least 93%, at least 94%, at least 95%, at least 96%, at least 97%, at least 98%, or at least 99%. In certain embodiments, compound 1 recovery at the later time point is from 90% to 110% of the compound 1 content at t=0.

Macroscopic appearance can be evaluated by, e.g., comparing the visual appearance of the pharmaceutical composition at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, substantially no change is observed in macroscopic appearance of the pharmaceutical composition at the later time point compared to the macroscopic appearance at t=0, e.g., no discoloration, no agglomeration, no change in opacity.

Microscopic appearance can be evaluated, e.g., by comparing the microscopic visualization (e.g., under 10× or 40× magnification) of the pharmaceutical composition at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, substantially no change in the microscopic appearance of the pharmaceutical composition is observed at the later time point, e.g., no observed agglomeration under magnification at the later time point compared to t=0. In certain embodiments less than 20% agglomeration under magnification is observed, such as, for example, less than 15%, less than 10%, or less than 5%.

Apparent pH of the pharmaceutical composition can be evaluated at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, the apparent pH is of the extracted solution portion of the composition, i.e., not the suspended composition. In certain embodiments, the apparent pH of the pharmaceutical composition changes by less than 1 pH unit at the later time point compared to the apparent pH at t=0, e.g., less than 0.5 pH unit.

Osmolality of the pharmaceutical composition can be evaluated at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, the osmolality of the pharmaceutical composition changes by less than 50 mOsm/kg at the later time point compared to the osmolality at t=0, e.g., less than 25 mOsm/kg.

Particle size distribution (D₉₀) of compound 1 in the pharmaceutical composition can be evaluated at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, the particle size distribution of compound 1 in the pharmaceutical composition changes by less than 50% at the later time point compared to the particle size distribution at t=0, e.g., less than 40%, less than 30%, less than 20%, less than 15%, less than 10%, or less than 5%.

Raman analysis of compound 1 in the pharmaceutical composition can be evaluated at a beginning time point (t=0) and later time point (e.g., 2 weeks, 4 weeks, 8 weeks, 4 months, 5 months, 6 months, 7 months, eight months, nine months, ten months, eleven months, or twelve months or more) under storage conditions. In certain embodiments, the polymorphic form of compound 1 in the pharmaceutical composition remains unchanged after storage conditions.

### 4.3. Methods

In one aspect, the present disclosure provides methods of treating obstructive sleep apnea comprising administering a pharmaceutical composition described herein to a subject intranasally.

In one aspect, the present disclosure provides pharmaceutical compositions as described herein for use in methods of treating obstructive sleep apnea, wherein said methods comprise administering a pharmaceutical composition described herein to a subject.

In certain embodiments, the subject self-administers the pharmaceutical composition.

In certain embodiments, the pharmaceutical composition is administered daily, e.g., prior to sleeping at night. In certain embodiments, the pharmaceutical composition is administered once prior to sleep. In other embodiments, the composition is administered twice or more prior to sleep.

In certain embodiments, the pharmaceutical composition described herein is administered to the subject while the subject is in an upright position, i.e., the subject's head elevation is 60° to 90° to the horizontal, e.g., from 600 to 80°, from 60° to 70°, from 70° to 90°, from 700 to 80°, or from 800 to 90°.

In other embodiments, the pharmaceutical composition described herein is administered to the subject while the subject's head is elevated to 450 to the horizontal, e.g., from 10° to 45°, from 20° to 45° from 30° to 45° from 40° to 45° from 20° to 45° from 20° to 40°, from 20° to 30°, from 30° to 45°, from 30° to 40°, or from 40° to 45°.

In certain embodiments, the pharmaceutical composition described herein is administered to the subject while the subject is in a supine position (head elevation 0° to the horizontal).

In certain embodiments, the subject assumes a supine position or ahead elevation of 45° or less to the horizontal following administration of the pharmaceutical composition, e.g., a head elevation from 10° to 45°, from 20° to 45°, from 30° to 45°, from 40° to 45°, from 20° to 45°, from 20° to 40°, from 20° to 30°, from 30° to 45°, from 30° to 40°, or from 40° to 45°. In certain embodiments, the subject assumes the post-administration position within 20 minutes of administration of the pharmaceutical composition, e.g., within 15 minutes, within 10 minutes, or within 5 minutes.

In certain embodiments, the pharmaceutical composition is administered to the subject while the subject is in an upright position and, following administration, the subject assumes a supine position or head position of 45° or less.

In certain embodiments, subject maintains the post-administration position for at least 4 hours following administration of the pharmaceutical composition, e.g., at least 5 hours, at least 6 hours, at least 7 hours, at least 8 hours, at least 9 hours, at least 10 hours, or at least 11 hours. In certain embodiments, “maintaining” the post-administration position allows for short (5 to 10 minute) deviations to vertical, e.g., for using the bathroom.

In certain embodiments, the pharmaceutical composition is administered to the subject while the subject is in an upright position and, following administration, the subject assumes a supine position or head position of 45° or less for 4 hours to 10 hours, e.g., 8 hours.

In certain embodiments, the pharmaceutical composition is administered to the subject from 5 minutes to 120 minutes prior to sleep, such as, for example, from 5 minutes to 100 minutes, from 5 minutes to 60 minutes, from 5 minutes to 30 minutes, from 5 minutes to 20 minutes, from 5 minutes to 15 minutes, from 5 to 10 minutes, from 10 minutes to 120 minutes, from 10 minutes to 100 minutes, from 10 minutes to 60 minutes, from 10 minutes to 30 minutes, from 10 minutes to 20 minutes, from 10 minutes to 15 minutes, from 15 minutes to 120 minutes, from 15 minutes to 100 minutes, from 15 minutes to 60 minutes, from 15 minutes to 30 minutes, from 15 minutes to 30 minutes, from 15 minutes to 20 minutes, from 20 minutes to 120 minutes, from 20 minutes to 100 minutes, from 20 minutes to 60 minutes, from 20 minutes to 30 minutes, from 30 minutes to 120 minutes, from 30 minutes to 100 minutes, from 30 minutes to 60 minutes, from 60 minutes to 120 minutes, from 60 minutes to 100 minutes, or from 100 minutes to 120 minutes. In certain embodiments, the pharmaceutical composition is administered to the subject from 10 minutes to 30 minutes prior to sleep.

In certain embodiments, intranasal administration of the pharmaceutical composition to the subject provides a total dose (i.e., the amount provided to both nostrils) from 0.1 mg to 100 mg of compound 1, such as, for example, from 0.1 mg to 75 mg, from 0.1 mg to 50 mg, from 0.1 mg to 25 mg, from 0.1 mg to 10 mg, from 5 mg to 100 mg, from 5 mg to 75 mg, from 5 mg to 50 mg, from 5 mg to 25 mg, from 5 mg to 10 mg, from 10 mg to 100 mg, from 10 mg to 75 mg, from 10 mg to 50 mg, from 10 mg to 25 mg, from 25 mg to 100 mg, from 25 mg to 75 mg, from 25 mg to 50 mg, from 50 mg to 100 mg, from 50 mg to 75 mg, or from 75 mg to 100 mg.

In certain embodiments, intranasal administration of the pharmaceutical composition to the subject provides a total dose (i.e., the amount provided to both nostrils) from 0.1 mg to 30 mg of compound 1, such as, for example, from 0.1 mg to 20 mg, from 0.1 mg to 10 mg, from 5 mg to 30 mg, from 5 mg to 25 mg, from 5 mg to 20 mg, from 5 mg to 15 mg, from 5 mg to 10 mg, from 10 mg to 30 mg, from 10 mg to 25 mg, from 10 mg to 20 mg, from 10 mg to 15 mg, from 15 mg to 30 mg, from 15 mg to 25 mg, from 15 mg to 20 mg, from 20 mg to 30 mg, from 20 mg to 25 mg, or from 25 mg to 30 mg.

In certain embodiments, administration of the pharmaceutical composition reduces upper-airway (UA) collapsibility for at least 4 hours following administration and during sleep, such as, for example, at least 4.5 hours, at least 5 hours, at least 5.5 hours, at least 6 hours, at least 6.5 hours, at least 7 hours, at least 7.5 hours at least 8 hours, at least 8.5 hours, at least 9 hours, at least 9.5 hours, or at least 10 hours. In certain embodiments, administration of the pharmaceutical composition inhibits UA collapsibility for 4 to 10 hours following administration and ruing sleep, such as, for example, 4 to 10 hours, 4 to 9 hours, 4 to 8 hours, 4 to 7 hours, 4 to 6 hours, 4 to 5 hours, 5 to 10 hours, 5 to 9 hours, 5 to 8 hours, 5 to 7 hours, 5 to 6 hours, 6 to 10 hours, 6 to 9 hours, 6 to 8 hours, 6 to 7 hours, 7 to 10 hours, 7 to 9 hours, 7 to 8 hours, 8 to 10 hours, 8 to 9 hours, or 9 to 10 hours.

In certain embodiments, inhibition of UA collapsibility can be measure by a reduction in pharyngeal critical closing pressure (Perit) compared to Pcrit measured for the same subject in the absence of administration of the composition. Pcrit can be measured as described in Osman, et al., “Topical Potassium Channel Blockage Improves Pharyngeal Collapsibility,” 163 #4 CHEST April 2023. In such embodiments, subjects are fitted with a nonvented nasal mask with a pressure transducer and a pneumotachograph to quantify airflow and mask pressure. A modified CPAP machine can be used to deliver transient pressure reductions to quantify UA collapsibility during sleep (pharyngeal critical closing pressure [Pcrit]).

In certain embodiments, administration of the pharmaceutical composition to the subject provides a Pcrit reduction of at least 2 cm H₂O for at least 4 hours, such as, for example, at least 2 cm H₂O for at least 4 hours, at least 2 cm H₂O for at least 4 hours for at least 6 hours, at least 3 cm H₂O for at least 4 hours, at least 3 cm H₂O for at least 6 hours, at least 4 cm H₂O for at least 4 hours, at least 4 cm H₂O for at least 6 hours, at least 5 cm H₂O for at least 4 hours, at least 5 cm H₂O for at least 6 hours, at least 6 cm H₂O for at least 4 hours, at least 6 cm H₂O for at least 6 hours, at least 7 cm H₂O for at least 4 hours, at least 7 cm H₂O for at least 6 hours, at least 8 cm H₂O for at least 4 hours, at least 8 cm H₂O for at least 6 hours, at least 9 cm H₂O for at least 4 hours, at least 9 cm H₂O for at least 6 hours, at least 10 or more cm H₂O for at least 4 hours, or at least 10 or more cm H₂O for at least 6 hours.

In certain embodiments, administration of the pharmaceutical composition to the subject provides a Pcrit reduction of at least 2 cm H₂O for at least 4 hours, such as, for example, at least 3 cm H₂O for at least 4 hours, at least 4 cm H₂O for at least 4 hours, at least 5 cm H₂O for at least 4 hours, at least 6 cm H₂O for at least 4 hours, at least 7 cm H₂O for at least 4 hours, at least 8 cm H₂O for at least 4 hours, at least 9 cm H₂O for at least 4 hours, or at least 10 or more cm H₂O for at least 4 hours

In certain embodiments, administration of the pharmaceutical composition to the subject provides a Pcrit reduction of at least 2 cm H₂O for at least 8 hours, such as, for example, at least 3 cm H₂O for at least 8 hours, at least 4 cm H₂O for at least 8 hours, at least 5 cm H₂O for at least 8 hours, at least 6 cm H₂O for at least 8 hours, at least 7 cm H₂O for at least 8 hours, at least 8 cm H₂O for at least 8 hours, at least 9 cm H₂O for at least 8 hours, or at least 10 or more cm H₂O for at least 8 hours.

In certain embodiments, the subject does not exhibit signs of irritation when the pharmaceutical composition is administered, e.g., reverse sneezing.

### 4.3.1. Drug-Device Combinations

In one aspect, the present disclosure provides a drug-device combination, e.g., a nasal delivery device comprising (i) a pharmaceutical composition described herein and (ii) a device suitable for intranasal delivery of the pharmaceutical composition to a subject.

In certain embodiments, the device comprises a nosepiece for fitting to a nostril of a subject and an actuation mechanism. Upon actuation of the actuation mechanism (e.g., by pressing or pushing), the device sprays composition out of a nozzle attached to the nosepiece and, provided the device is situated within the nostril of a subject, delivers the pharmaceutical composition to the nasal airway of the subject.

In certain embodiments, the device comprises a fluid dispenser pump and a reservoir as described in EP 2139605, incorporated by reference herein in its entirety. In certain embodiments, the pump is an atmospheric pump. In certain embodiments, the reservoir comprises the pharmaceutical composition described herein. In certain embodiments, the pump of the fluid dispenser is positioned on a neck of the reservoir. In certain embodiments, the pump of the fluid dispenser is attached on the neck of the reservoir via one or more fastener rings. In certain embodiments, the one or more fastener rings comprise screw-on rings, snap-on rings, crimpable caps, or any combination thereof. In certain embodiments, the one or more fastener rings secures the pump to the neck of the reservoir. In certain embodiments, the one or more fastener rings comprise a compression region. In certain embodiments, the one or more fastener rings are attached to the neck of the reservoir and the pump by applying pressure on the compression region.

In certain embodiments, the pump comprises a piston. In certain embodiments, the piston is located inside of the pump. In certain embodiments, the piston moves between two positions: a rest position and a dispensing position. In certain embodiments, the piston is at the rest position when the composition is not being dispensed from the device. In certain embodiments, the piston is at the dispensing position when the composition is being dispensed from the device. In certain embodiments, the piston slides within the pump from the rest position to the dispensing position upon each use of the device to dispense the composition from the reservoir.

In certain embodiments, the piston is connected to an actuator. In certain embodiments, the actuator is a rod. In certain embodiments, the actuator moves between two positions: a rest position and a dispensing position. In certain embodiments, the actuator comprises a dispenser head. In certain embodiments, the actuator head is the location where the composition is dispensed. In certain embodiments, the actuator comprises a top portion and a bottom portion. In certain embodiments, the dispenser head is located in the top portion of the actuator. In certain embodiments, the actuator further comprises a pusher. In certain embodiments, the pusher is located in the bottom portion of the actuator. In certain embodiments, the user of the device directly or indirectly applies pressure to the pusher to cause the pump to dispense the composition. In certain embodiments, the user applies pressure to the pusher to move the actuator from the rest position to the dispensing position. In certain embodiments, the user applies pressure to the pusher to move the piston from the rest position to the dispensing position. In certain embodiments, the composition is dispensed when the actuator is in the dispensing position. In certain embodiments, the composition is dispensed when the piston is in a dispensing position. In certain embodiments, when the actuator is moved into the dispensing position, an amount of the composition moves from the reservoir through the actuator to the actuator head. In certain embodiments, when the amount of the composition from the reservoir is dispensed, a corresponding amount of air from outside of the device is provided to the reservoir. In certain embodiments, the amount of the composition dispensed is a predetermined amount of the composition. In certain embodiments, the same amount of composition is dispensed in each use of the device.

In certain embodiments, the pump further comprises a seal. In certain embodiments, seal comprises a ferrule. In certain embodiments, when the actuator is in the rest position, the seal is in contact with the bottom portion of the actuator. In certain embodiments, when the actuator is in the rest position, the seal is not in contact with the top portion of the actuator. In certain embodiments, when the actuator is in the dispensing position, the seal is not contact with the bottom portion of the actuator. In certain embodiments, when the actuator is in the dispensing position, the seal is in contact with the top portion of the actuator. In certain embodiments, the seal attaches to a fastener. In certain embodiments, the fastener is a snap fastener, a screw fastener, or both. In certain embodiments, the fastener is a fastener ring. In certain embodiments, the fastener attaches the seal to the neck of the reservoir. In certain embodiments, the seal prevents any of the composition from leaking out of the reservoir. In certain embodiments, the pump further comprises a gasket. In certain embodiments, the gasket is configured to attach the seal to the fastener. In certain embodiments, the vent path of the air entering the pump can flow from the seal to the pump and then to the reservoir.

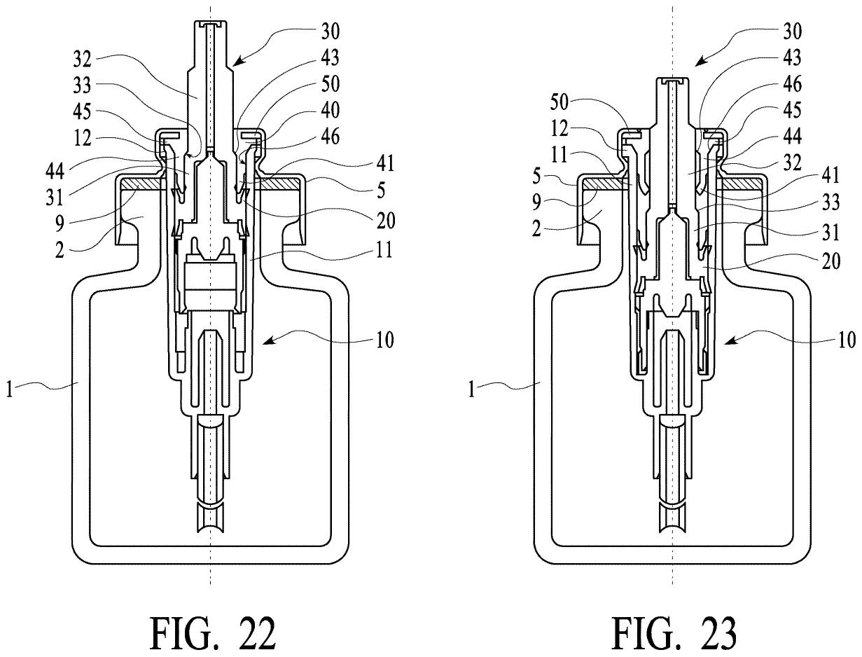

An exemplary device is shown in FIG. 22 and FIG. 23. The device comprises a reservoir 1 provided with a neck 2, a pump 10 being assembled on said neck 2 of the reservoir 1 by means of a fixing ring 5, preferably with the interposition of a seal 9, called seal of col. The pump comprises a pump body 11 provided with an upper end edge 12. A piston 20 slides sealingly inside said pump body 11 at each actuation to dispense a dose of product from the tank 1. In a conventional manner, the piston 20 is integral, in particular one-piece, as shown in the drawings, with an actuating rod 30 on which a dispensing head or pusher (not shown) assembles on which the user will directly or indirectly to operate the pump. A ferrule 40 is assembled inside the pump body 11 and cooperates with the piston 20 to define the rest position thereof. Ferrule 40 supports a seal 50 adapted to achieve the seal between the ferrule 40 and the fixing ring 5. This fixing ring 5 can be any, including crimpable as shown in the drawings, but it could also be snap-on, screwable or any other suitable fixing ring.

Ferrule 40 cooperates sealingly with the actuating rod 30 when the actuating rod moves between the rest position and an intermediate position, and said ferrule 40 no longer cooperates sealingly with the actuating rod 30 between said intermediate position and the actuating position represented on the FIG. 23. Thus, when the cooperation between the ferrule 40 and the actuating rod 30 is no longer sealed, the venting system is open in a conventional manner.

In certain embodiments, an exemplary device comprises comprising a reservoir 1 containing a pharmaceutical composition described herein and a pump 10, said pump 10 being assembled on said reservoir 1 by means of a fastener ring 5, wherein the fluid pump 10 comprises a pump body 11, a piston 20 that slides in leaktight manner in said pump body 11 between a rest position and an actuated position, said piston 20 being secured to an actuator rod 30 that extends axially out from said pump body 11, a ferrule 40 being mounted on the top edge 12 of said pump body 11, said ferrule 40 defining the rest position of said piston 20, the pump being characterized in that said ferrule 40 co-operates in leaktight manner with the actuator rod 30 while the actuator rod 30 is moving between the rest position and an intermediate position, and said ferrule 40 co-operating in non-leaktight manner with the actuator rod 30 while the actuator rod 30 is moving between said intermediate position and the actuated position, so as to enable the pump 10 to be vented, said piston 20 not being in contact with said ferrule 40, when in the rest position.

In certain embodiments, the nasal device delivers from 50 μl to 150 μL of the pharmaceutical composition per actuation of the device, such as, for example, from 50 μl to 125 μl, from 50 μl to 100 μl, from 50 μl to 75 μl, from 75 μl to 150 μl, from 75 μl to 125 μl, from 75 μl to 100 μl, from 100 μl to 150 μl, from 100 μl to 125 μl, or from 125 μl to 150 μl.

In certain embodiments, the nasal device delivers from 85 mg to 130 mg of the pharmaceutical composition per actuation of the device, such as, for example, from 85 mg to 120 mg, from 85 mg to 115 mg, from 85 mg to 110 mg, from 85 mg to 100 mg, from 90 mg to 130 mg, from 90 mg to 120 mg, from 90 mg to 115 mg, from 90 mg to 110 mg, from 90 mg to 100 mg, from 95 mg to 130 mg, from 95 mg to 120 mg, from 95 mg to 115 mg, from 95 mg to 110 mg, from 95 mg to 100 mg, from 100 mg to 130 mg, from 100 mg to 120 mg, from 100 mg to 115 mg, from 100 mg to 110 mg, from 110 mg to 130 mg, from 110 mg to 120 mg, or from 120 mg to 130 mg. In certain embodiments, the nasal device delivers from 100 mg to 120 mg or from 105 mg to 120 mg of the pharmaceutical composition per actuation of the device.

In certain embodiments, the standard deviation of the amount of pharmaceutical composition between actuations of the device is less than 10, such as, for example, less than 8, less than 5, or less than 3.

Depending on the pharmaceutical composition and nature of the nasal delivery device, the spray produced upon actuation can be a jet spray or a mist spray.

In certain embodiments, the device provides a jet spray of the pharmaceutical composition upon actuation. In certain embodiments, the jet spray comprises droplets of the composition having droplet sizes from 100 μm to 1,000 μm, such as, for example, from 100 μm to 750 μm, from 100 μm to 500 μm, from 100 μm to 250 μm, from 250 μm to 1,000 μm, from 250 μm to 750 μm, from 250 μm to 500 μm, from 500 μm to 1,000 μm, from 500 μm to 750 μm, or from 750 μm to 1,000 μm. In certain embodiments, the jet spray comprises droplets of the pharmaceutical composition having droplet sizes from about 300 μm to 500 μm. In certain embodiments, the jet spray comprises droplets of the composition having droplet sizes from 700 μm to 1,000 μm.

In certain embodiments, the device provides a mist spray of the pharmaceutical composition upon actuation. In certain embodiments, the mist spray comprises droplets of the pharmaceutical composition having droplet sizes from 50 μm to 1,000 μm, such as, for example, from 50 μm to 750 μm, from 50 μm to 500 μm, from 50 μm to 250 μm, from 100 μm to 1,000 μm, from 100 μm to 750 μm, from 100 μm to 500 μm, from 100 μm to 250 μm, from 250 μm to 1,000 μm, from 250 μm to 750 μm, from 250 μm to 500 μm, from 500 μm to 1,000 μm, from 500 μm to 750 μm, or from 750 μm to 1,000 μm. In certain embodiments, the mist spray comprises droplets of the pharmaceutical composition having droplet sizes from 70 μm to 150 μm, from 80 μm to 150 μm, or from 90 μm to 120 μm. In certain embodiments, the mist spray comprises droplet sizes of the pharmaceutical composition from 80 μm to 100 μm, e.g., 80 μm to 90 μm.

In certain embodiments, the device delivers from 50 μl to 300 μl of the pharmaceutical composition per actuation of the device, e.g., to the nasal airway of the subject, such as, for example, from 50 μl to 250 μl, from 50 μl to 200 μl, from 50 μl to 150 μl, from 50 μl to 100 μl, from 100 μl to 300 μl, from 100 μl to 250 μl, from 100 μl to 200 μl, from 100 μl to 150 μl, from 150 μl to 300 μl, from 150 μl to 250 μl, from 150 μl to 200 μl, from 200 μl to 300 μl, or from 250 μl to 300 μl.

The dose of compound 1 delivered to the patient (including all actuations and administration to both nostrils) will vary depending on the wt % of compound 1 in the pharmaceutical composition and the number of actuations administered.

In certain embodiments, the nasal device delivers from 9 mg to 11 mg of compound 1 per 100 μl of pharmaceutical composition actuated from the device, such as, for example, from 9 mg to 10.5 mg, from 9 mg to 10 mg, from 9 mg to 9.5 mg, from 9.5 mg to 11 mg, from 9.5 mg to 10.5 mg, from 9.5 mg to 10 mg, from 10 mg to 11 mg, from 10 mg to 10.5 mg, or from 10.5 mg to 11 mg. In certain embodiments, the nasal device delivers from 9 to 10 mg or 10 to 1.5 mg of compound 1 per 100 μl of pharmaceutical composition actuated from the device.

In certain embodiments, the pharmaceutical composition is administered into a single nostril by actuation of the nasal device to provide the total dose of compound 1 to the subject. In certain embodiments, the device can be actuated one or more times to provide the total dose of compound 1 to the subject, such as, for example, two times, three times, four times, or five or more times.

In certain embodiments, the pharmaceutical composition is administered into each nostril by actuation of the nasal device to provide the total dose of compound 1 to the subject. In certain embodiments, the device can be actuated one or more times to provide the total dose of compound 1 to the subject, such as, for example, two times, three times, four times, or five or more times.

In certain embodiments, one or more priming sprays can be performed prior to administration. Such priming sprays remove the air from the pump and actuator tubing, replacing with formulation ready for dosing as initial actuations may contain low product weights. Such priming sprays are not actuated into the nostril of the subject and therefore do not provide compound 1 to the subject. In certain embodiments, one, two three, or four or more priming sprays are performed prior to administration of the composition to the nostril of the subject.

### 4.4. Enumerated Embodiments

1. A pharmaceutical composition comprising:
   2′-{[2-(4-Methoxy-phenyl)-acetylamino]-methyl}-biphenyl-2-carboxylic acid (2-pyridin-3-yl-ethyl)-amide (compound 1) or a pharmaceutically acceptable salt thereof and
   a mucoadhesive polymer,
   wherein compound 1 is present in an amount greater than 0.25% (w/w).
2. The composition of embodiment 1, wherein compound 1 is present in an amount from greater than 0.25% (w/w) to 20% (w/w).
3. The composition of embodiment 1 or 2, wherein compound 1 is present in an amount from 3% (w/w) to 10% (w/w).
4. The composition of embodiment 3, wherein compound 1 is present in an amount of about 6% (w/w).
5. The composition of embodiment 3, wherein compound 1 is present in an amount of about 9% (w/w).
6. The composition of any one of embodiments 1-5, wherein the mucoadhesive polymer comprises a polyacrylic acid derivative, cellulose derivative, natural polymer, polyvinyl pyrrolidone (PVP), dextran polymer, polyethylene oxide polymer, thermoreversible polymer, ionic responsive polymer, copolymer of polymethyl vinyl ether and maleic anhydride, or a combination thereof.
7. The composition of embodiment 6, wherein the mucoadhesive polymer comprises a cellulose derivative selected from a hydroxy alkyl cellulose polymer, methyl cellulose polymer, carboxymethyl cellulose (CMC) polymer, a salt of carboxymethyl cellulose, or a combination thereof.
8. The composition of embodiment 7, wherein the mucoadhesive polymer comprises a hydroxy alkyl cellulose polymer selected from hydroxypropyl methylcellulose (HPMC) and hydroxypropyl cellulose (HPC).
9. The composition of embodiment 8, wherein the mucoadhesive polymer comprises HPMC.
10. The composition of embodiment 6, wherein the mucoadhesive polymer is a cellulose derivative selected from a hydroxy alkyl cellulose polymer, methyl cellulose polymer, carboxymethyl cellulose (CMC) polymer, a salt of carboxymethyl cellulose, or a combination thereof.
11. The composition of embodiment 10, wherein the mucoadhesive polymer is a hydroxy alkyl cellulose polymer selected from hydroxypropyl methylcellulose (HPMC) and hydroxypropyl cellulose (HPC).
12. The composition of embodiment 11, wherein the mucoadhesive polymer is HPMC.
13. The composition of embodiment 7, wherein the mucoadhesive polymer comprises a methyl cellulose polymer.
14. The composition of embodiment 7, wherein the mucoadhesive polymer is a methyl cellulose polymer.
15. The composition of embodiment 6, wherein the mucoadhesive polymer comprises a natural polymer selected from an arabic gum, tragacanth gum, agar polymer, xanthan gum, copolymer of alginic acid and sodium alginate, chitosan polymer, pectin, carrageenan, pullulan polymer, modified starch, or a combination thereof.
16. The composition of embodiment 15, wherein the mucoadhesive polymer comprises carrageenan.
17. The composition of embodiment 6, wherein the mucoadhesive polymer is a natural polymer selected from an arabic gum, tragacanth gum, agar polymer, xanthan gum, copolymer of alginic acid and sodium alginate, chitosan polymer, pectin, carrageenan, pullulan polymer, modified starch, or a combination thereof.
18. The composition of embodiment 17, wherein the mucoadhesive polymer is a carrageenan.
19. The composition of any one of embodiments 1-18, wherein the mucoadhesive polymer is present in an amount from 0.1% (w/w) to 20% (w/w).
20. The composition of embodiment 19, wherein the mucoadhesive polymer is present in an amount from 0.3% (w/w) to 1% (w/w), preferably from 0.3% (w/w) to 0.6% (w/w).
21. The composition of any one of embodiments 1-20, wherein the composition is free of microcrystalline cellulose.
22. The composition of embodiment 1, wherein the mucoadhesive polymer does not comprise microcrystalline cellulose.
23. The composition of any one of embodiments 1-23, further comprising a tonicity modifier.
24. The composition of embodiment 23, wherein the tonicity modifier is selected from dextrose, lactose, sodium chloride, calcium chloride, magnesium chloride, sorbitol, sucrose, mannitol, trehalose, raffinose, polyethylene glycol, hydroxyethyl starch, glycine, and combinations thereof.
25. The composition of embodiment 24, wherein the tonicity modifier is selected from sorbitol, mannitol, and a combination thereof.
26. The composition of any one of embodiments 23-25, wherein the tonicity modifier is present in an amount from 0.1% (w/w) to 10% (w/w), preferably from 0.1% (w/w) to 5% (w/w).
27. The composition of embodiment 26, wherein the tonicity modifier is present in an amount from 1% (w/w) to 30% (w/w).
28. The composition of any one of embodiments 1-27, further comprising a wetting agent.
29. The composition of embodiment 28, wherein the wetting agent is selected from a polysorbate, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), a reaction product of castor oil and ethylene oxide, and combinations thereof.
30. The composition of embodiment 29, wherein the wetting agent is selected from a polysorbate and a reaction product of castor oil and ethylene oxide.
31. The composition of embodiment 30, wherein the wetting agent is a polysorbate, wherein the polysorbate is polysorbate 80.
32. The composition of embodiment 30, wherein the wetting agent is a reaction product of castor oil and ethylene oxide selected from polyoxyl 35 castor oil and polyoxyl 40 hydrogenated castor oil.
33. The composition of any one of embodiments 28-32, wherein the wetting agent is present in an amount from 0.1% (w/w) to 5.0% (w/w).
34. The composition of embodiment 33, wherein the wetting agent is present in an amount from 0.1% (w/w) to 1.0% (w/w), preferably 0.3% (w/w) to 0.6% (w/w).
35. The composition of any one of embodiments 1-34, further comprising an antimicrobial preservative.
36. The composition of embodiment 35, wherein the antimicrobial preservative is selected from chlorhexidine gluconate, phenyl ethyl alcohol, 1-phenoxyethanol, benzyl alcohol, sorbic acid, thimerosal, phenylmercuric acetate, a benzoate, a paraben, a sorbate, and combinations thereof.
37. The composition of embodiment 36, wherein the antimicrobial preservative is potassium sorbate.
38. The composition of any one of embodiments 35-37, wherein the antimicrobial preservative is present in an amount from 0.01% (w/w) to 1% (w/w).
39. The composition of embodiments 38, wherein the antimicrobial preservative is present in an amount from 0.05% (w/w) to 0.15% (w/w).
40. The composition of any one of embodiments 1-39, further comprising an aqueous vehicle.
41. The composition of embodiment 40, wherein the aqueous vehicle comprises deionized water, saline, phosphate buffer, citrate buffer, malate buffer, tartrate buffer, balanced salt solution, salts of organic acids, combinations of organic acids and salts of organic acids (e.g., tribasic sodium citrate and citric acid, malic acid and sodium malate, and potassium sodium tartrate and tartaric acid) or a combination thereof.
42. The composition of embodiment 41, wherein the aqueous vehicle comprises citrate buffer and phosphate buffer.
43. The composition of any one of embodiments 40-42, wherein the composition comprises aqueous vehicle in an amount from 85% (w/w) to 95% (w/w).
44. The composition of embodiment 43, wherein the composition comprises aqueous vehicle in an amount from 85% (w/w) to 90% (w/w).
45. The composition of any one of embodiments 1-44, further comprising EDTA.
46. The composition of embodiment 45, wherein the composition comprises EDTA in an amount from 0.01% (w/w) to 2% (w/w).
47. The composition of embodiment 1, wherein the composition comprises:
   from 0.25% (w/w) to 20% (w/w) compound 1,
   from 0.1% (w/w) to 20% (w/w) mucoadhesive polymer,
   from 0.1% (w/w) to 10% (w/w) tonicity modifier,
   from 0.1% (w/w) to 50% (w/w) wetting agent,
   from 0.01% (w/w) to 1% (w/w) antimicrobial preservative, and
   from 85% (w/w) to 95% (w/w) aqueous vehicle.
48. The composition of embodiment 47, wherein the composition comprises:
   from 3% (w/w) to 10% (w/w) compound 1,
   from 0.3% (w/w) to 0.6% (w/w) mucoadhesive polymer,
   from 1% (w/w) to 3% (w/w) tonicity modifier,
   from 0.3% (w/w) to 0.6% (w/w) (w/w) wetting agent,
   from 0.05% (w/w) to 0.15% (w/w) antimicrobial preservative; and
   from 85% (w/w) to 90% (w/w) aqueous vehicle.
49. The composition of embodiment 47 or 48, wherein the mucoadhesive polymer is methyl cellulose, the tonicity modifier is mannitol, the wetting agent is polyoxyl 40 hydrogenated castor oil, the antimicrobial preservative is potassium sorbate, and the aqueous vehicle is a mixture of citrate buffer and phosphate buffer.
50. The composition of embodiment 47 or 48, wherein the mucoadhesive polymer is HPMC, the tonicity modifier is sorbitol, the wetting agent is polyoxyl 35 castor oil, the antimicrobial preservative is potassium sorbate, and the aqueous vehicle is a mixture of citrate buffer and phosphate buffer.
51. The composition of embodiment 47 or 48, wherein the mucoadhesive polymer is carrageenan, the tonicity modifier is sorbitol, the wetting agent is polyoxyl 35 castor oil, the antimicrobial preservative is potassium sorbate, and the aqueous vehicle is a mixture of citrate buffer and phosphate buffer.
52. The composition of embodiment 51, further comprising EDTA in an amount from 0.01% (w/w) to 2% (w/w).
53. The composition of any one of embodiments 1-52, wherein the composition is in the form of a liquid.
54. The composition of embodiment 53, wherein the liquid is a suspension.
55. The composition of any one of embodiments 1-54, wherein the composition has an apparent pH from 4 to 9, preferably about from 5 to 7, more preferably about 6.
56. The composition of any one of embodiments 1-55, wherein the composition has an osmolality from 200 mOsm/kg to 500 mOsm/kg, more preferably from 250 mOsm/kg to 350 mOsm/kg, more preferably about 300 mOsm/kg.
57. The composition of any one of embodiments 1-56, wherein the composition has a compound 1 particle size distribution D₉₀ from 5 μm to 30 μm, preferably from 5 μm to 15 μm.
58. The composition of embodiment 57, wherein the composition has a compound 1 bimodal particle size distribution D₉₀.
59. The composition of embodiment 58, wherein from 25% (w/w) to 75% (w/w) of compound 1 has a D₉₀ from 20 μm to 30 μm and the remainder of compound 1 has a D₉₀ from 5 μm to 15 μm.
60. The composition of any one of embodiments 1-59, wherein less than 0.1% (w/w) of compound 1 is in solution.
61. The composition of any one of embodiments 1-60, wherein the composition is stable when stored at 25° C. for 2 weeks as determined by one or more of the following: (i) compound 1 recovery, (ii) macroscopic appearance, (iii) microscopic appearance, (iv) apparent pH, (v) osmolality, (vi) particle size distribution (D₉₀), and Raman analysis.
62. The composition of any one of embodiments 1-61, wherein the composition is stable when stored at 40° C. for 2 weeks as determined by one or more of the following: (i) compound 1 recovery, (ii) macroscopic appearance, (iii) microscopic appearance, (iv) apparent pH, (v) osmolality, (vi) particle size distribution (D₉₀), and (vii) Raman analysis.
63. The composition of any one of embodiments 1-62, wherein the composition is stable when stored at 25° C. for 4 weeks as determined by one or more of the following: (i) compound 1 recovery, (ii) macroscopic appearance, (iii) microscopic appearance, (iv) apparent pH, (v) osmolality, (vi) particle size distribution (D₉₀), and (vii) Raman analysis.
64. The composition of any one of embodiments 1-63, wherein the composition is stable when stored at 40° C. for 4 weeks as determined by one or more of the following: (i) compound 1 recovery, (ii) macroscopic appearance, (iii) microscopic appearance, (iv) apparent pH, (v) osmolality, (vi) particle size distribution (D₉₀), and (vii) Raman analysis.
65. The composition of any one of embodiments 1-64, wherein when the composition is administered intranasally to a subject suffering from obstructive sleep apnea prior to commencing sleep, the composition inhibits upper-airway collapsibility for at least 6 hours following administration and during sleep as measured by a reduction in pharyngeal critical closing pressure (Perit) compared to Perit measured for the same subject in the absence of administration of the composition.
66. The composition of embodiment 65, wherein P_{crit} is reduced by at least 2 cm H₂O for at least 6 hours.
67. The composition of embodiment 65 or 66, wherein P_{crit} is reduced by at least 2 to 10 cm H₂O for at least 6 hours.
68. The composition of any one of embodiments 65-67, wherein P_{crit} is reduced for at least 8 hours.
69. A nasal delivery device comprising the pharmaceutical composition of any one of embodiment 1-68.
70. The nasal delivery device of embodiment 69, wherein the device comprises a nosepiece for fitting to a nostril of a subject and an actuation mechanism, wherein the nosepiece includes a nozzle through which the composition is delivered as a spray into the nasal airway of the subject upon actuation of the actuation mechanism.
71. The nasal delivery device of embodiment 69 or 70, wherein a volume of 50 μl to 150 μl of the composition is delivered per actuation.
72. The nasal delivery device of embodiment 69 or 70, wherein the nozzle provides a jet spray of the composition upon actuation.
73. The nasal delivery device of embodiment 72, wherein the jet spray comprises droplets having a droplet size from 100 μm to 1,000 μm, preferably greater than 400 μm.
74. The nasal delivery device of embodiment 69 or 70, wherein the nozzle provides a mist spray of the composition upon actuation.
75. The nasal delivery device of embodiment 74, wherein the mist spray comprises droplets having a droplet size from 50 μm to 1,000 μm, preferably from about 80 μm to about 150 μm, more preferably from 90 μm to 120 μm.
76. A method for treating obstructive sleep apnea in a subject in need thereof, the method comprising administering an effective amount of the pharmaceutical composition of any one of embodiment 1-68 to the subject prior to sleep.
77. The method of embodiment 76, wherein the composition is administered intranasally.
78. The method of embodiment 77, wherein the composition is administered intranasally by a nasal delivery device of any one of embodiment 69-75.
79. The method of embodiment 77 or 78, wherein the composition is administered once (one actuation per nostril) or twice (two actuations per nostril) prior to sleep.
80. The method of any one of embodiments 76-79, wherein the composition is administered from 5 minutes to 120 minutes prior to sleep, preferably from 10 minutes to 30 minutes.
81. The method of any one of embodiments 76-80, wherein upper-airway collapsibility is inhibited for at least 6 hours following administration and during sleep as measured by a reduction in pharyngeal critical closing pressure (Perit) compared to P_{crit} measured for the same subject in the absence of administration of the composition.
82. The method of embodiment 81, wherein P_{crit} is reduced by at least 2 cm H₂O for at least 6 hours.
83. The method of embodiment 81 or 82, wherein P_{crit} is reduced by at least 2 to 10 cm H₂O for at least 6 hours.
84. The method of any one of embodiments 81-83, wherein P_{crit} is reduced for at least 8 hours.
85. The method of any one of embodiments 76-84, wherein the subject does not experience reverse sneezing upon administration of the composition.

### 4.5. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### 4.5.1. Example 1—A Phase 2 Clinical Trial Demonstrated No Significant Improvement in Obstructive Sleep Apnea after Intranasal Administration of Compound 1

Given initial preclinical evidence that AVE-118 (compound 1) shifted the mechanoreceptor response threshold for the genioglossus muscle to more positive pressures and dose-dependently inhibited upper airway collapsibility in anesthetized pigs (Wirth et al., SLEEP, 36(5):699-708 (2013)), a formulation suitable for intranasal administration to human subjects was prepared and a phase 2 clinical trial was conducted.

### 4.5.1.1 Study Formulation

The intranasal formulation used in the study (“Ph2 formulation”) is set forth in Table 1 below. The formulation is a suspension (30 mg/mL, equivalent to 3 wt % compound 1) using micronized API with a mean particle size of about 4 μm. The composition was similar to marketed suspensions for nasal application like Nasacort®. It contained the following excipients:
benzalkonium chloride (preservative)
dispersible cellulose (microcrystalline cellulose (MCC)/carmellose sodium) (for stabilization, viscosity enhancement and prevention from agglomeration)
mannitol (for isotonization).

Benzalkonium chloride is a standard excipient for nasal drugs and was used in the suspension at usual concentrations needed for preservation.

TABLE 1

| Components | Level in final formulation (% w/w) |
|---|---|
| Compound 1 | 2.92 |
| Mannitol | 0.49 |
| Benzalkonium chloride | 0.0019 |
| Microcrystalline cellulose CL611 | 0.17 |
| Water for injection | 96.41 |
| Total | 100.00 |

Studies on the tolerability of the formulation performed by DSE in dogs confirmed sufficient tolerability as a prerequisite to start human clinical trials.

### 4.5.1.2 Study Design

This was a single intranasal dose, multi-center, randomized, placebo-controlled, double-blind, 2-way crossover study with two treatment periods in male patients with Obstructive Sleep Apnea-Hypopnea Syndrome (OSAHS).

After a screening phase of up to 21 days, the patients received single doses of 18 mg of compound 1 administered as 3 actuations of 100 μL of study drug into each nostril (9 mg/nostril) or matching placebo at bedtime in each study period. The single nasal doses were administered in a sitting position. There was a wash-out period of 2 days to 3 weeks between the two study periods. The duration of the study per patient ranged from 13 days to a maximum of 8 weeks.

Efficacy was assessed in a blinded fashion using overnight polysomnographic recordings (PSG) which were centrally read and interpreted. The central readers were blinded with regard to the treatment sequence the patients received and with regard to potential safety findings. Maintenance of Wakefulness Tests (MWT) and Stanford Sleepiness Scores (SSS) were also used for the assessment of Excessive Daytime Sleepiness (EDS), and plasma concentrations of compound 1 were assessed at baseline and approximately 10 hours after study drug administration (data not shown).

Thirty-eight patients (19 patients per sequence group) were planned to be randomized in this study (taking into account a 10% drop-out rate). Patients were centrally randomized through an Interactive Voice Response System (IVRS) to one of the 2 sequence groups (sequence 1: Placebo/Compound 1; sequence 2: Compound 1/Placebo), using a pre-established randomization list of randomly permuted blocks of size 4 with balanced allocation (1:1 ratio) between the 2 sequences. Randomization was stratified by center.

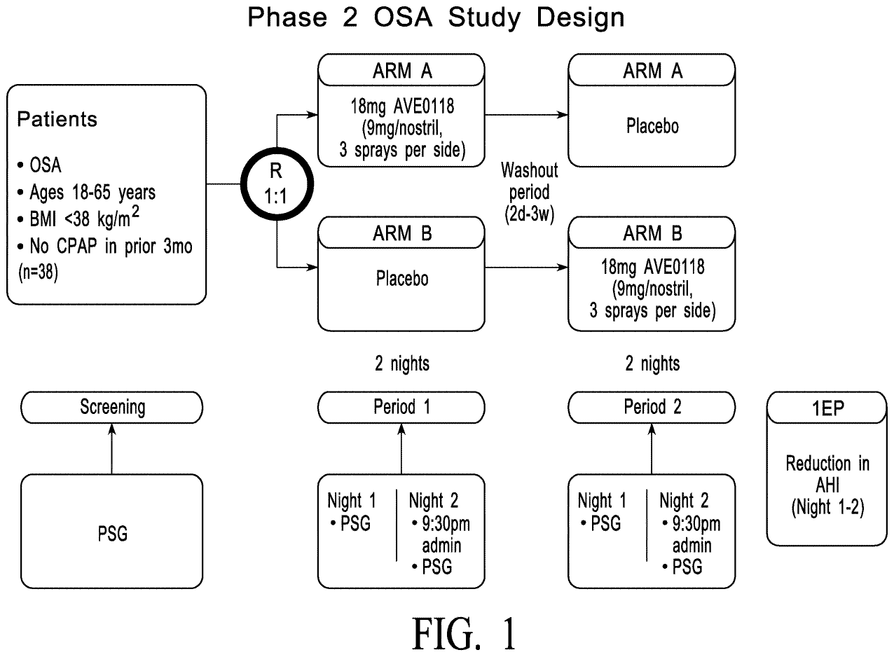

A schematic representation of the study design is presented in FIG. 1.

### 4.5.1.3 Primary Endpoint

The primary efficacy endpoint was the change from baseline in the Apnea Hypopnea Index (AHI) (number/hour), defined as the number of apnea-hypopnea events divided by the Total Sleep Time (TST); baseline was defined as the last available value before study drug intake in each period.

### 4.5.1.4 Key Secondary Endpoints

The change from baseline of AHI in the first 4 hours of the night (num/hr) was a key secondary endpoint as it was aimed to explore a potential early effect of the drug. AHI in the first 4 hours of the night was evaluated as the number of apnea-hypopnea events occurring during the 4 hours following treatment intake, divided by TST during that period.

### 4.5.1.5 Definition of Analysis Populations

### Randomized Patients

Randomized patients were defined as all patients for whom a treatment kit number was allocated through the randomization process and recorded in the IVRS database.

### Intention to Treat Population

The intention to treat (ITT) population was defined as all randomized patients who took at least one dose of study medication, and had a baseline and a post-baseline AHI assessment for at least one treatment period. Patients were analyzed using the treatment they actually received at each period, irrespective of the treatment sequence they were randomized to.

### Per Protocol Population

The Per-Protocol (PP) population was a subset of the Intent-To-Treat (ITT) population and was defined as all randomized patients who were evaluable for the primary efficacy criterion and had no major efficacy-related protocol deviations. Patients were evaluable for the primary criterion if they had a baseline and a post-baseline AHI assessment for at least one treatment period, with a minimum TST of 4 hours for each assessment and a maximum change of TST from baseline to post-baseline of 25%. In PP analyses, only evaluable AHI measurements were taken into account (measurements with minimum TST of 4 hours for each exam within a period and maximum change of TST from baseline to post-baseline of 25%). Patients were analyzed using the treatment they actually received at each period.

### Safety Population

The Safety population was defined as all randomized patients exposed to the study medication, regardless of the amount of treatment administered. Patients were analyzed using the treatment they actually received at each period.

### 4.5.1.6 Primary Efficacy Analysis

Efficacy analyses were performed on the PP and ITT populations. Treatment differences in the change from baseline to Day 1 in AHI were evaluated through a linear mixed effects model for crossover design, with fixed effects for treatment, period, sequence and center, with random term for patient (nested within sequence), and with baseline AHI as covariate, using SAS PROC MIXED.

The Least Square (LS) mean of Compound 1 versus placebo difference in the change from baseline was provided with its 95% confidence interval. Statistical tests were two-sided with a type I error fixed at 5%.

### 4.5.1.7 Results

### 4.5.1.7.1 Patient Accountability

All 38 randomized patients were included in the ITT, PK and Safety populations.

Five patients were excluded from the PP analysis for the following reasons:
2 patients had an AHI greater than 72 at inclusion (subjects 250001073 and 25002005);
3 patients had less than 60% of obstructive or mixed Apnea Hypopnea (AH) events at inclusion (subjects 250001051, 250002007 and 25002009).

Patient 250001034 was randomized to the treatment sequence “AVE0118/Placebo” but received the treatment sequence “Placebo/AVE0118”. Given the early phase of development of the study, he was analyzed using the treatment he actually received at each period in the ITT, PP, PK and Safety populations.

Baseline demographics are shown in Table 2.

TABLE 2

| | n = 38 | |
|---|---|---|
| Age, years | 51.5 | (9.5, 27-64) |
| Male Sex | 100% | |
| Race | 36 (94.7%) Caucasian/white | |
| Weight | 86.5 kg | (12.8, 64-118) |
| BMI (kg/m²) | 28.3 | (4, 21-38) |
| AHI, events/hr | 34.6 | (16, 15-68) |

### 4.5.1.7.2 Study Disposition

All 38 randomized patients completed the study as planned, and received the planned total dose of 18 mg of compound 1.

### 4.5.1.7.3 Efficacy

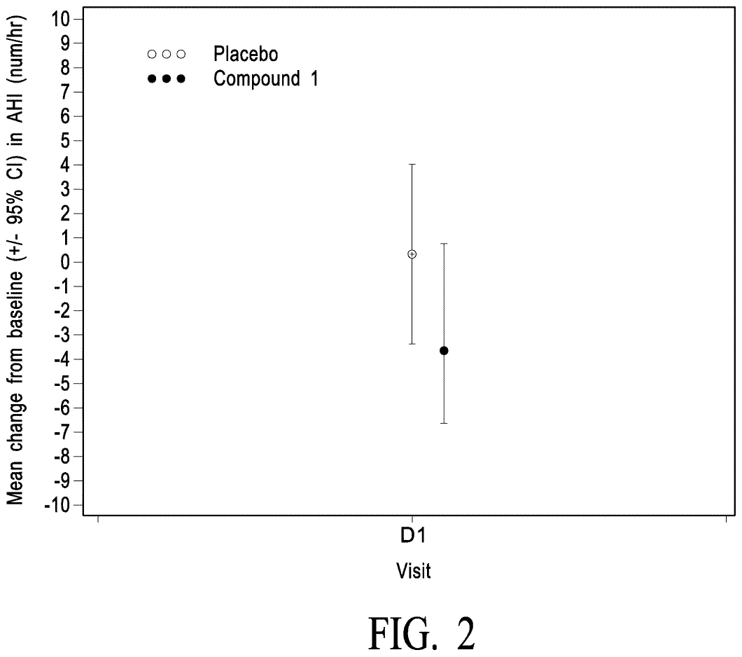

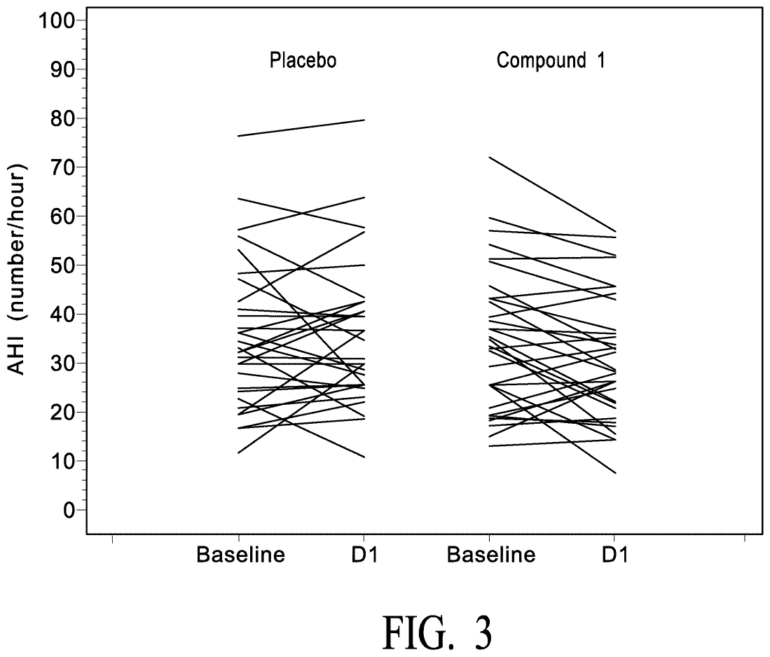

FIG. 2 plots mean change from baseline in AHI (+/−95 confidence interval) in the per-protocol (PP) population, where baseline is defined as the last available value before drug intake in each period. Note that only evaluable measurements were taken into account (TST≥4 h and absolute change in TST≤25% within a period). FIG. 3 is a plot of individual values of AHI in the per protocol population. Results are summarized in Table 3 below.

TABLE 3

| | AHI (num/hr) | 95% confidence | |
|---|---|---|---|
| Population analyzed | LS mean difference | interval | P-value |
| Per Protocol = 33 | −4.00 (−14.5%) | −8.31 to 0.31 | 0.0682 |
| ITT | −3.78 | −9.07 to 1.52 | 0.1597 |

In the Per Protocol population, there was a trend towards a higher reduction of AHI with a single administration of compound 1 compared to placebo; however, this reduction was not statistically significant (p=0.0682). The observed treatment difference was only 4.00 num/hr (95% CI −8.31 to 0.31) in favor of compound 1. This corresponded to a treatment difference on the percent change from baseline of 14.75% in favor of compound 1 (95% CI between 2.13 and 31.63), which was far lower than the expected 40% reduction targeted in the clinical study protocol.

Results in the ITT population and from the sensitivity analysis based on square root transformation were in line with these findings, with even higher p-values (lower statistical significance) observed in the ITT population than in the PP population.

### 4.5.1.8 Safety

Treatment emergent adverse events (TEAE) are summarized in Table 4.

TABLE 4
All grade TEAEs (>5% frequency)

| n | Placebo | Compound 1 | |
|---|---|---|---|
| (%) | (n = 38) | (n = 38) | |
| Respiratory | 1 (2.6%) | 37 | (97.4%) |
| Sneezing | 0 | 32 | (84.2%) |
| Cough | 1 (2.6%) | 19 | (50.0%) |
| Nasal discomfort | 0 | 14 | (36.8%) |
| Throat irritations | 0 | 13 | (34.2%) |
| Rhinorrhea | 0 | 7 | (18.4%) |
| Eye Disorders | 1 (2.6%) | 22 | (57.9%) |
| Increased tearing | 1 (2.6%) | 20 | (52.6%) |

### 4.5.2. Example 2—Assessment of Phase 2 Trial Failure

A large number of possible factors could have contributed to the failure of the human phase 2 trial despite promising animal model data. Possible explanations, which are not mutually exclusive and not exhaustive, include:
1. Problems with the animal model
   a. The anesthetized pig OSA animal model used by Wirth 2013 does not adequately recapitulate human disease, and therefore does not predict efficacy in humans
      i. at the limit, the efficacy signal in the pig model does not predict efficacy in humans and the drug might never work in humans
2. Problems with the design and/or implementation of the clinical protocol
   a. the plasma PK in the human phase 2 trial was not reflective of the slow release profile seen in the pig study
   b. problems with administration technique
      i. inconsistent angle
      ii. inconsistent insertion depth
   c. insufficient time may have been allowed for the drug to work before the clinical benefit was measured; the drug may need to be given earlier or the patient may need to start treatment a few days before they see any clinical benefit
   d. there is a large intra-patient night-to-night variability in apnea-hypopnea index (AHI) rate (depends on how well a person sleeps, how much REM sleep they get, how much time on their back vs. side); the trial may have been insufficiently powered to account for this
   e. trial subjects may have had pathological features resulting in damage to the actual drug target (if the nerves no longer function or are of reduced quantity, then the K+ channel blocking mechanism is not going to have any effect)
   f. trial subjects may have had etiological traits preventing this mechanism of action being relevant in these patients (high loop gain, more collapsible airway, etc.)
   g. poor adherence to the clinical protocol
3. Incorrect Dose
   a. Dose does not translate from pig to human (no doses above 18 mg tested in man)
   b. Response variability patient to patient (some require 18 mg some require 36 mg etc)
4. Drug stability
   a. The drug was not physically or chemically stable in the formulation/device prior to application such that the selected dose was not achieved
   b. The drug lost its potency upon application to the nasal mucosa
5. Drug delivery
   a. the device+formulation was suboptimal in terms of dose delivered to the target site/site of action/nasal pharynx (i.e., too much drug was inhaled, ingested, exhaled) for the following reasons:
      i. Particle and droplet size was suboptimal
      ii. Too high a volume was actuated up each nostril in too shorter time
      iii. Aggregation of the formulation particles causing its removal (sneezing) or suboptimal dissolution
      iv. Particle size caused enhanced mucociliary clearance
      v. Device/formulation not actuated at optimum position
      vi. The drug particles stuck to the mucosal surface and got no further/allowing no dissolution and therefore absorption
b. Quantity of drug deposition on the nasal tissue
   i. Too little drug deposited
      1. Too little drug administered (18 mg dose is too low)
      2. Too much inhaled
      3. Too much exhaled
      4. Too much swallowed
      5. The formulation was not able to deposit the drug effectively:
         a. Drug particle aggregation might have defeated uniform dispersion on the tissue surface
         b. Improper adhesion to the epithelial surface
         i. Too much adhesion in the wrong place may prevent drug ever reaching the target, estimated to be predominately in the nasopharynx
         c. Formulation didn't allow the drug to penetrate the mucus layer to reach the epithelial surface
c. Location of drug deposition
   i. Spray device (plume geometry) delivered drug to an ineffective location (or to the lungs or too far back such that the drug was swallowed)
   ii. Spray droplets too large or too small to be transported by airflow in the nasal passage to the right location
      1. Small droplets could be inhaled and would also account of the rapid Cₘₐₓ on the plasma PK profile
   iii. Spray droplets prior to deposition on nasal mucosa too large or too small to be transported by the epithelial mucus to the nasopharynx target before the drug is deposited on the epithelial surface (and can no longer be moved)
   iv. Drug particle size distribution and or agglomeration
      1. differences in location of deposition for different particle sizes
d. Duration of deposition
   i. Drug particles removed by “normal” mucus flow
   ii. Drug particles removed by triggered “clearance mechanisms” (extra mucus production, sneezing, coughing) due to local irritation
   iii. Drug particles dissolving too fast (average particle size too small or the proportion of small particles was too great i.e. wrong particle size distribution)
   iv. drug particle size distribution and/or agglomeration—larger particles are cleared faster by mucus
6. Drug retention
   a. Quantity of drug may have been sufficient for a short duration of effect but insufficient for an effect for several hours or even 8 hours
      i. Drug may have been washed away by the flow of mucus
      ii. Drug may have dissolved too quickly
      iii. Drug may have permeated into the bloodstream too quickly
      iv. Retention at the site of action was suboptimal for the above reasons and thus sustained/controlled delivery was not achievable over the 8 h window
7. Tissue permeation
   a. Drug unable to cross the surface barrier of the epithelium
   b. Drug not retained at the target site
   c. Drug unable to diffuse laterally in the epithelial tissue to reach nerve endings of pressure sensing nerves (drug target)
      i. Have sufficient dispersion/permeation to reach a threshold percentage of nerves to have the desired pharmacological effect
   d. Aggregation of the drug particles may have hampered dissolution and reduced the bio-availability of drug in solution to permeate into the tissue
   e. formulation may have permitted too high a flux, resulting in systemic uptake rather than local tissue distribution
   f. Formulation may not have permitted a high enough and consistent enough flux of drug into the tissue
      i. Did not maintain the correct buffer conditions to control the release of the drug from the particles (too acidic and drug may dissolve too quickly)
      ii. formulation not optimized to saturation solubility of the active compound in order to maximize thermodynamic driving force for permeation
g. Absorption across the mucosal epithelium was suboptimal due to—
   i. insufficient rate of drug dissolution
   ii. The drug's inherent impermeability or suboptimal flux to achieve the required drug levels at the pressure-sensing nerves to achieve the required increase in tone and prevention of collapsibility over the required duration to achieve clinical benefit
   iii. suboptimal equilibrium between the drug in suspension and drug in solution in the nasal mucosal fluids to achieve required dissolution and therefore absorption
8. The surface area of pressure-sensing nerves with bound drug was too low to have an effect on the negative pressure reflex
   a. Drug clumping together
   b. Device not delivering a uniform spray
   c. No lateral diffusion of drug in the tissue
   d. Delivery did not cover a sufficient fraction of the of the nasopharynx surface area
      i. binding/retention to the mucosal epithelium e.g. different mucosal layers and nasal epithelium meaning it could not permeate to act on the pressure sensing nerves to increase tone and prevent collapsibility
9. Device
   a. formulation and device were not compatible
   b. inconsistent delivery of dose
   c. inconsistent spray geometry.

### 4.5.3. Example 3—Confirmation of Translational Relevance of Pig OSA Model

To rule out the possibility that the anesthetized pig model used in Wirth 2013 had provided translationally false positive results with nasal administration of compound 1 (Example 1), the following experiment was performed.

Rosenberg and colleagues recently reported a successful phase 2 clinical trial of a combination of a norepinephrine reuptake inhibitor, atomoxetine, and the antimuscarinic drug, aroxybutynin, the enantiomerically pure R isomer of oxybutynin, in treating obstructive sleep apnea. (Rosenberg et al., J. Clin. Sleep Med. 18(12):2837-2844, 2022). Treated subjects had a statistically significant and clinically meaningful difference from placebo in hypoxic burden. A study was thus performed to assess efficacy of atomoxetine and oxybutynin in the pig model.

### 4.5.3.1 Methods

The principles of the method of this study are described in detail in Wirth et al., SLEEP, Vol. 36, No. 5, 2013. All studies in animals were conducted in accordance with German laws for protection of animals. Male castrated German Landrace pigs (weight range 20-35 kg) were used.

### Sedation

To avoid stress responses during ear vein cannulation for the induction of general anesthesia, pigs were sedated in the animal house by intramuscular injection of a mixture of 3 ml Rompun® 2% (Xylazine HCl, 23.3 mg/mL; Bayer), and 7 ml Zoletil® 100 (Virbac). The contents of a vial of Zoletil®, (250 mg of tiletamine, and 250 mg of zolazepam) as dry powder and 3 ml Rompun were dissolved in 7 mL of the vehicle. Next, 0.1 ml/kg of this solution was injected intramuscularly.

### Anesthesia

Experiments were performed under general anesthesia induced and maintained by a mixture of α-chloralose and urethane.

After sedation of the pigs, an ear vein was cannulated. Before general anesthesia was started two vials Novaminsulfon (Zentiva®, 2 g Metamizol-Natrium 1 H₂O) and 2 vials Atropine (Atropinsulfat B. Braun) to reduce saliva were administered intravenously at the beginning of the experiment and 3 hours later, and analgesia was supported by 0.04 mg/kg Novaminsulfon intravenously during the whole experimental setup.

Anesthesia was induced by administration of a 20 mL bolus of α-Chloralose solution (25 g α-Chloralose (Sigma®)±12.5 g Borax Anhydrous ad 600 ml saline, Sigma®) intravenously into an ear vein corresponding to a dose of 0.028 g/kg for a pig of 30 kg body weight followed by a 20 mL bolus of urethane solution (100 g/500 mL Urethane, Sigma®; corresponding to a dose of 0.133 g/kg). Urethane was dissolved in saline (0.9%). Chloralose was dissolved in saline that contained borax.

Before skin incisions were made bupivacaine 0.5% JENAPHARM®, having a long-lasting anesthetic effect, was injected for additional infiltration anesthesia. The incisions were closed and sutured to prevent drying.

Next a femoral vein was cannulated by using a percutaneous introducer set and anesthesia was maintained by continuous infusion of 1 ml/kg/h of urethane- and α-chloralose solutions mixed in a 50-mL syringe that was filled with 40 mL of the urethane solution and 10 mL of α-chloralose solution, corresponding to a dose of 0.07 g/kg/h Chloralose and 4 g/kg/h Urethane into the femoral vein.

Anesthesia was monitored via heart rate, blood pressure, ventilation, electrocardiogram (ECG), determination of blood gases, pulse oximetry (tail), and regular reflex testing for pain. Reflex testing was performed 15 min before each collapsibility test by cornea reflex sensitivity. In case of insufficient anesthesia, the pig would blink and a bolus dose of 5 mL of the anesthesia maintenance solution would then be applied and, if needed, repeated until this reflex disappeared. Body temperature was monitored and maintained using an infrared lamp. Oxygen was applied if necessary to keep oxygen saturation close to 95% via a tube placed in front of the outlet of the pneumotachometer attached to the facial mask. In this open system a flow rate of 2 L/min of oxygen was sufficient to keep saturation close to 95%.

### Surgery

A tracheotomy was performed 1-2 cm below the larynx. Care was taken to avoid injury of the laryngeal nerves. Two tracheal tubes (Mallinckrodt™ 11.5 mm OD, 8.5 mm ID) were inserted into the trachea, one into the rostral part and the other into the caudal part of the trachea so that they could be fixed by a thread around the trachea to seal the connection. Using a T-shaped connection piece, the rostral cannula was connected to a tube to the negative pressure device and to the distal tracheal cannula. The distal tracheal cannula was additionally connected to a tube with an open end to atmosphere via a T-shaped connection piece that served for free tracheal breathing, circumventing the UA. By appropriate opening and clamping of these tubes breathing could be switched from nasal breathing to breathing through the caudal tracheal cannula, circumventing the UA, and the (isolated) UA could be connected to the negative pressure device, causing airflow in the inspiratory direction. A thin tube was advanced into the rostral trachea tubing system and connected to a pressure transducer element (MPX Type 399/2 Hugo Sachs Electronic-Havard-Apparatus) for the measurement of tracheal (sublaryngeal) pressure.

The snout was sealed with adhesive tapes, sparing the nostrils, and covered with a silicon bag (upper half of a breathing bag 0.5 l) onto which a pneumotachometer (SN 370-1491 Hans Rudolph, Inc.) was fixed at the free end. The pneumotachometer was connected to a differential pressure transducer (Type 381, Hugo Sachs Electronic-Harvard Apparatus) for the measurement of nasal airflow. The part of the bag on the pig's snout was sealed airtight by an elastic ribbon.

The pneumotachometer was calibrated using the calibration factor of the manufacturer and additional with a rotameter with defined airstreams in the expected range, the differential pressure transducer was calibrated by using a pressure calibration device (Gauer). Calibration configurations were stored and checked monthly.

All biological signals were recorded by a Hugo Sachs Plugsys-amplifier system and continuously stored on a computer hard disk by an online data acquisition and analysis system (Hem-evolution 4.3 Notocord Systems, Croissy-sur-Seine, France). Data backups were made every 24 hours by a Langmeyer backup system.

### Removal of Mucus in the Upper Airway

A manual resuscitator for human use applying mild negative pressure by means of a suction tube, introduced through the distal end of the tracheal tube system, and forwarded to the nostrils and slowly withdrawn, was used to remove mucus from the upper airways. The procedure was repeated if necessary, during the whole experiment, except during the first 30 min after administration of test compounds.

In case of heavy mucus production, a lavage of the mucosa from the upper airways was made with isotonic saline. After switching from nasal breathing to lung breathing with the nose in a vertical position, 50-ml of isotonic (0.9%) saline heated to 37° C. was instilled slowly through the nostrils to flow out of the distal tracheal tube. This procedure was repeated until the nasal lavage fluid was clear and free of any particles or visible mucus. The manual resuscitator was used to remove all lavage fluid from the upper airways.

### Genioglossus EMG Measurement

For the bipolar registration of the GG EMG, steel needles were placed 4-6 mm deep through the mylohyoid muscle into the GG muscle via a small skin incision midway between the chin and the hyoid bone. The raw EMG signals were amplified, filtered (bandwidth 50 Hz to 10 kHz), rectified, and integrated (moving average with a time constant of 1 sec) using a Hugo Sachs EMG-amplifier.

### Collapsibility Tests by Application of Negative Pressure

The negative pressure device consisted of a negative pressure container (50 L) with a manometer that was evacuated by a vacuum pump and activated via a solenoid valve. The device enabled generation of any negative pressure as low as −150 cm H₂O. The pressure level was set and the desired level reached in the device before the pressure challenge. To induce a UA collapse (collapsibility test), breathing was switched from nasal to tracheal breathing. Actuation of the negative pressure device applied the preselected device pressure onto the UA airway via the tube connecting the device to the cranial tracheal cannula. Since the UA dilating muscles were only activated during inspiration both nostrils were closed with two fingers to increase nasal resistance during expiration and opened again during inspiration. Negative pressure was applied for at least three breaths, which caused a collapse of the UA under control conditions as indicated by the measurements of airflow (to the device) and of sublaryngeal pressure. In pigs such negative pressure challenges were performed with negative pressures of −50, −100, and −150 cm H₂O. The highest pressure of −50 cm H₂O was applied first and maintained for at least three breaths. The next pressure challenge applying the increasingly negative pressures were performed after pauses of at least ten breaths. A complete collapsibility test at the three pressure levels indicated was performed before administration of the test compound and at regular intervals after administration (up to 420 min after administration of the test compound).

In the non-treated situation, the UA would remain collapsed in the inspiratory phase. In case of effective stimulation of UA dilatory muscle activity by the test drug the UA would open during the inspiratory phase to close again during expiration as in the control situation because, physiologically, there is no expiratory UA-dilating muscle activity but the UA is continuously exposed to the negative pressure (and re-open during the next inspiration). Opening of the UA during inspiration is indicated by an inspiratory rise in the sublaryngeal pressure approaching atmospheric pressure and by the resumption of airflow during inspiration in the isolated UA segment.

To differentiate a real collapse from a paradox collapse (sucking of surrounding tissue of the UA or clogged by mucus), a collapse is indicated by an increase of negative pressure of 90% from baseline of the adjusted device pressure (Δ P) and a decrease of peak inspiratory airflow to nearly zero ml/sec.

### Intravenous Administration of Test Compound

Atomoxetine and Oxybutynin were dissolved in a vehicle of the following composition:
10% Ethanol
40% Polyethylenglycol
50% Auqua dest.

The volume of the bolus administration was 0.1 ml/kg and subsequent infusion rate was 1 ml/kg/h.

Two anesthetized pigs were used in this study. At time zero pigs received intravenous administration of 1 mg/kg Atomoxetine given as a bolus over 1 min immediately followed by a continuous infusion of 0.275 mg/kg/h. 10 min later a 20 μg/kg bolus of Oxybutynin was administered and 12.5 μg/kg/h were immediately and continuously infused for 4 hours.

### Euthanasia:

At the end of the experiments pigs were euthanized without recovering from anesthesia with an i.v. bolus of T61.

### 4.5.3.2 Results

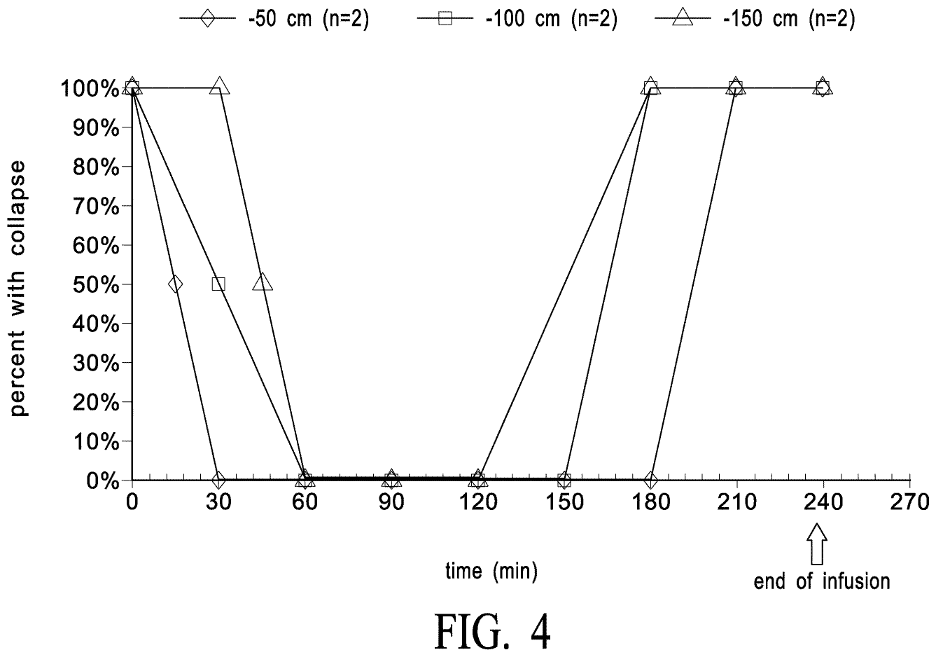

The drug combination provided complete inhibition of upper airway collapsibility with a delay of 60 min after bolus injection. At timepoint 30 min, inhibition of collapsibility was still incomplete. Efficacy lasted until timepoint 120 min at pressure level −150 cm H₂O, until 150 min at pressure level −100 cm H₂O and until 180 min at pressure level −50 cm H₂O (FIG. 4, n=2) although the drug combination was continuously infused over 4 hours. The need for active metabolite formation for Oxybutynin, species differences in metabolism and differences in PK and metabolism as a consequence of the different modes of administration (oral administration in man versus i.v. administration in pigs) may have had an influence on the time profile of efficacy in this investigation.

A marked increase in heart rate and a moderate increase in blood pressure and respiratory rate were observed after administration of the combination. Pigs became restless transiently (moving hind legs) after administration of the combination of Atomoxetine and Oxybutynin. No changes in genioglossus EMG measurement were observed after administration of the drugs.

### 4.5.3.3 Conclusion

In this pig study of upper airway collapsibility, the combination of atomoxetine plus racemic oxybutynin, with proven clinical efficacy in OSA patients (Rosenberg et al., 2022), showed full inhibition of upper airway collapsibility. The onset of action and time to full efficacy was delayed by 60 min under more stringent negative pressure conditions of −100 cm and −150 cm H₂O. Loss of efficacy occurred during the 4-hour continuous infusion of the drug combination. Three hours after the start of the bolus administration (plus subsequent maintenance infusion) collapsibility had returned at all pressures tested despite continuous infusion.

In conclusion, the combination of Atomoxetine and Oxybutynin, for which clinical efficacy in human clinical OSA studies has been demonstrated (Rosenberg et al., 2022), showed full inhibition of upper airway collapsibility in this pig model, although efficacy was lost during continuous infusion, strongly suggesting that the infusion doses used in this investigation were too low to maintain efficacy. Similarly to the AD109 clinical trial reported by Rosenberg, the systemic side effects on heart rate and blood pressure limited the dosing possibilities in this study, providing further evidence that the pig model is translationally relevant.

These data demonstrate that the pig model is translationally relevant to predicting efficacy in human OSA, with the caveat that these results were obtained using drugs administered intravenously rather than topically via nasal administration.

### 4.5.4. Example 4—Solution Formulation has Greater Duration of Efficacy than Suspension Formulation

### 4.5.4.1 Summary

To test whether failure of the phase 2 trial of Example 1 could instead be attributed to use of a suspension formulation of compound 1, the efficacy of a solution formulation was compared to efficacy of the phase 2 suspension formulation in the pig OSA model.

### 4.5.4.2 Methods

Methods used were essentially as described in Example 3 with certain modifications.

### 4.5.4.3 Formulations, Preparation and Storage of Test Compounds

Preparation of compound 1 solution: compound 1 was dissolved in a vehicle containing 5% DMSO and 95% PEG 400. 18.75 mg compound 1 was dissolved in 5 ml vehicle, providing a stock solution of 3.75 mg/ml. The opalescent solution was stirred at room temperature with a magnetic stirrer until it was completely dissolved and stored at 4° C. in a brown glass vial protected from light. Fresh dilutions of the stock solution were made just before administration of the test compound. For a dose of 0.3 mg/animal, a factor 10 dilution was made with vehicle. Applying 400 μl into each nostril of this solution yielded a dose of 0.3 mg per pig. Applying 400 μl into each nostril of the stock solution yielded a dose of 3 mg per pig.

Preparation of compound 1 suspension: compound 1 was received either as ready to use formulation in a concentration of 3% w/w or as diluent which was used for compounding the suspension on the day of use by adding micronized compound 1 to diluent to prepare a compound 1 concentration of 2.25% w/w (Table 5). 67.5 mg of compound 1 was suspended in 3 ml vehicle at the first day of administration. The suspensions were stored at room temperature in brown glass vials protected from light in a box (50 days). Suspensions prepared from solid substance were freshly prepared just for the time of administration.

In the laboratory, where the pig experiments were performed, all suspension formulations were stirred at 1000 rpm with a magnetic stirrer (Heidolph®) for one hour, followed by sonication (HBM®) for 10 min and shaken by hand immediately until administration. The suspensions were visually checked for homogeneity just before nasal administration. Then the compound was delivered with an Eppendorf-pipette at a volume of 200 or 400 μl into each nostril depending on the experiment, yielding a dose of 12 or 24 mg/pig. The 18 mg dose was administered in a volume of 400 μl per nostril. The vertical position of the nose allowed the test compound to flow to all parts of the pharynx.

TABLE 5

| | 30 mg/mL | vehicle |
|---|---|---|
| | formulation | formulation |
| Substance | (% w/w) | (% w/w) |
| Compound 1 | 3.00 | — |
| Water | 96.33 | 99.31 |
| Mannitol | 0.49 | 0.51 |
| Benzalkonium chloride | 0.002 | 0.002 |
| Microcrystalline cellulose | 0.17 | 0.18 |
| Total | 100.00 | 100.00 |

### 4.5.4.4 Results

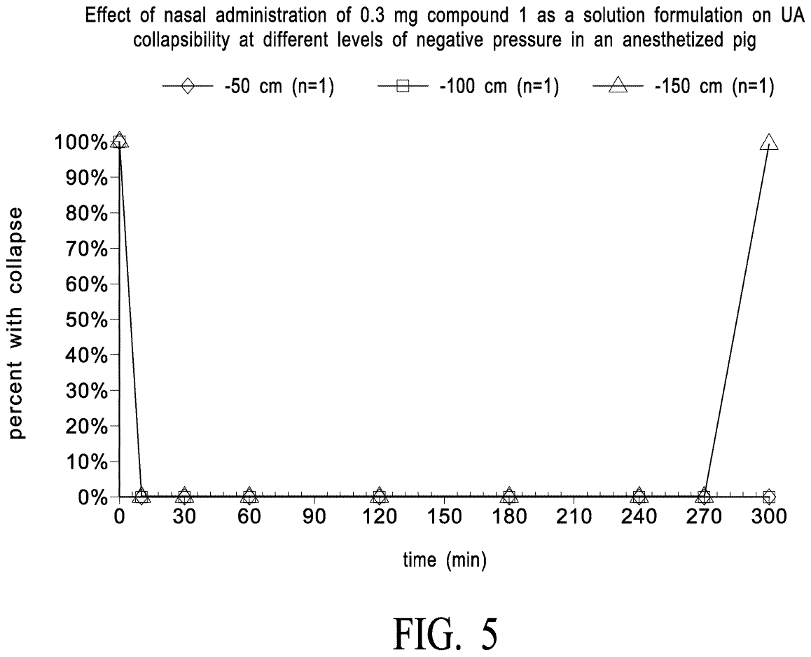

In a vertical position of the pig, a total of 0.3 mg (400 μl per nostril) of the compound 1 solution formulation applied nasally showed full inhibition of UA collapsibility at all collapse levels from time point 10 to time point 270 min, whereas at collapse levels of −50 and −100 cm H₂O, inhibition exceeded the end of observation time which was terminated at 300 min. (Table 6, FIG. 5, n=2).

TABLE 6
percentage of pigs showing upper airway collapsibility
Time (min)

| Negative | | | | | | | | | |
|---|---|---|---|---|---|---|---|---|---|
| Pressure | | | | | | | | | |
| (cm H₂O) | | | | | | | | | |
| (n = 1) | 0 | 10 | 30 | 60 | 120 | 180 | 240 | 270 | 300 |
| −50 | 100% | 0% | 0% | 0% | 0% | 0% | 0% | 0% | 0% |
| −100 | 100% | 0% | 0% | 0% | 0% | 0% | 0% | 0% | 0% |
| −150 | 100% | 0% | 0% | 0% | 0% | 0% | 0% | 0% | 100% |

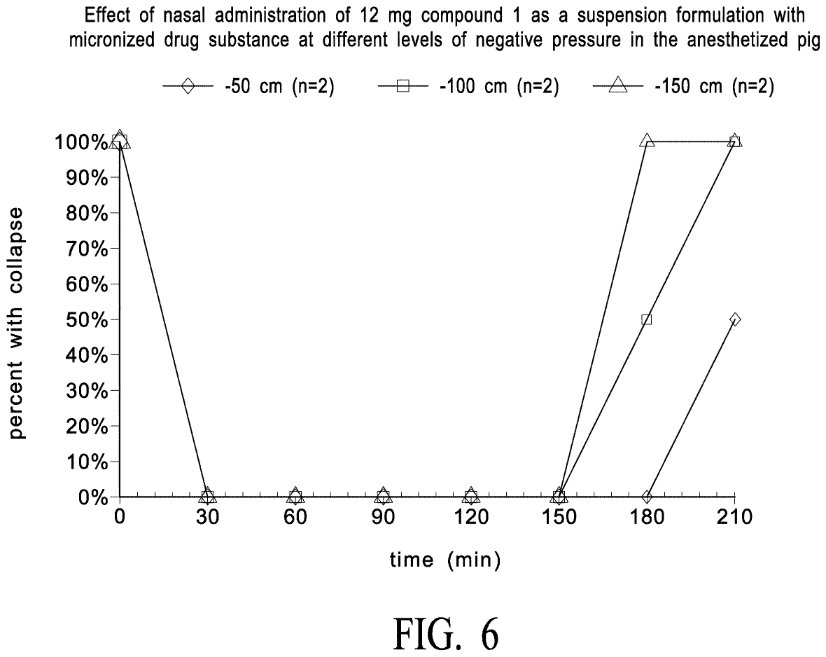

The compound 1 suspension formulation (12 mg, 200 μl/nostril) applied nasally showed full inhibition of UA collapsibility at all collapse levels from time point 30 to time point 150 min, whereas at collapse level of −50 cm H₂O, inhibition prolonged until timepoint 180 min. (Table 7, FIG. 6, n=2.)

TABLE 7
percentage of pigs showing upper airway collapsibility
Time (min)

| Negative | | | | | | | | | |
|---|---|---|---|---|---|---|---|---|---|
| Pressure | | | | | | | | | |
| (cm H₂O) | | | | | | | | | |
| (n = 1) | | 0 | 30 | 60 | 90 | 120 | 150 | 180 | 210 |
| −50 | Mean | 100% | 0% | 0% | 0% | 0% | 0% | 0% | 50% |
| | SEM | 0% | 0% | 0% | 0% | 0% | 0% | 0% | 50% |
| | n | 2 | 2 | 2 | 2 | 2 | 2 | 2 | 2 |
| −100 | Mean | 100% | 0% | 0% | 0% | 0% | 0% | 50% | 100% |
| | SEM | 0% | 0% | 0% | 0% | 0% | 0% | 50% | 0% |
| | n | 2 | 2 | 2 | 2 | 2 | 2 | 2 | 2 |
| −150 | Mean | 100% | 0% | 0% | 0% | 0% | 0% | 100% | 100% |
| | SEM | 0% | 0% | 0% | 0% | 0% | 0% | 0% | 0% |
| | n | 2 | 2 | 2 | 2 | 2 | 2 | 2 | 2 |

These results demonstrate that a solution formulation of compound 1 has a greater duration of efficacy than the suspension formulation in the failed Phase 2 study of Example 1.

### 4.5.5. Example 5—but Only the Suspension Formulation Maintains Upper Airway Collapsibility Following Nasal Lavage

### 4.5.5.1 Summary

The results of Example 4 suggested that failure of the phase 2 trial could be attributed, at least in part, to the use of a suspension rather than solution formulation of compound 1. To better model duration of efficacy in human patients during post-administration flow of mucus during recumbency and sleep, the following experiment was conducted.

Resistance to upper airway collapsibility following nasal lavage was tested in the pig collapsibility model of inhibition of upper airway collapsibility (OSA model) after nasal administration of compound 1, formulated as either the solution or suspension formulations described in Example 4, followed by nasal lavage to model post-administration mucus flow.

### 4.5.5.2 Methods

Methods used were used as adapted from those described in Example 4.

To test whether the effect of the test compound on collapsibility in different formulations can be removed by nasal lavage, isotonic saline heated to 37° C. was applied to the upper airways. The fluid was instilled slowly through the nostrils to flow out of the distal tracheal tube. A manual resuscitator was used to remove the rest of lavage fluid from the upper airways.

The nasal lavages were performed at different timepoints after nasal drug administration and with different volumes of saline as shown in Table 8:

TABLE 8

| | | | 3% (w/w) | 3% (w/w) | |
|---|---|---|---|---|---|
| | Solution | Solution | Suspension | Suspension | 2.25% (w/w) |
| | Formulation | Formulation | 24 mg | 24 mg | Suspension |
| Nasal Lavage | 0.3 mg | 3 mg | Pig 1 | Pig 2 | 18 mg |
| Saline | 50 | 50 | 2 × 10 and | 3 × 50 each | 50 |
| Volume (ml) | | | 1 × 20 | | |
| Timepoints of | 60 | 60 | 60; 90 and | 30; 90 and | 1 |
| lavage(s) after | | | 120 | 120 | |
| administration | | | | | |
| (min) | | | | | |
| Effect on | abolished | abolished | not | not | not |
| collapsibility | | | abolished | abolished | abolished |

### 4.5.5.3 Results

### Solution Formulations:

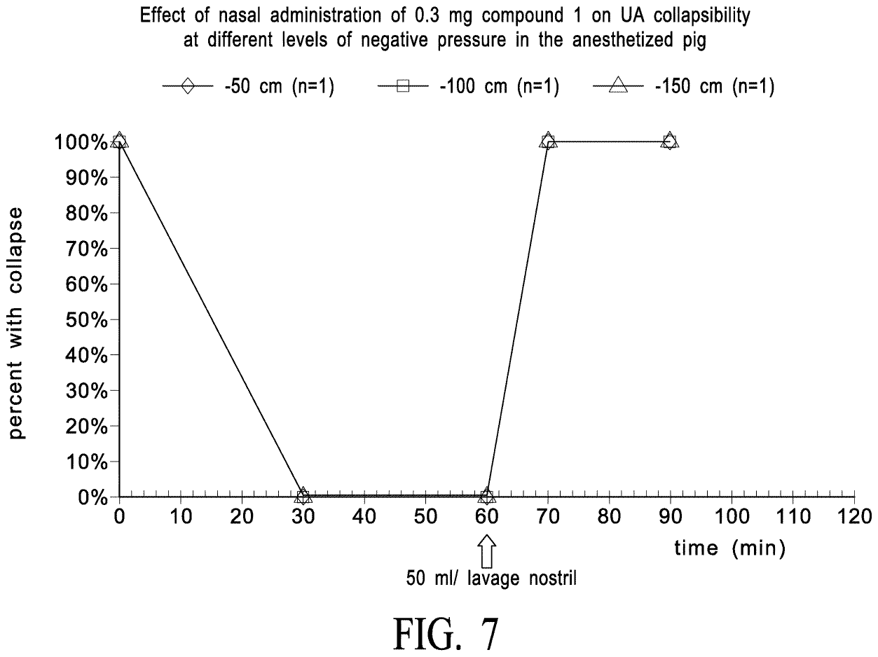

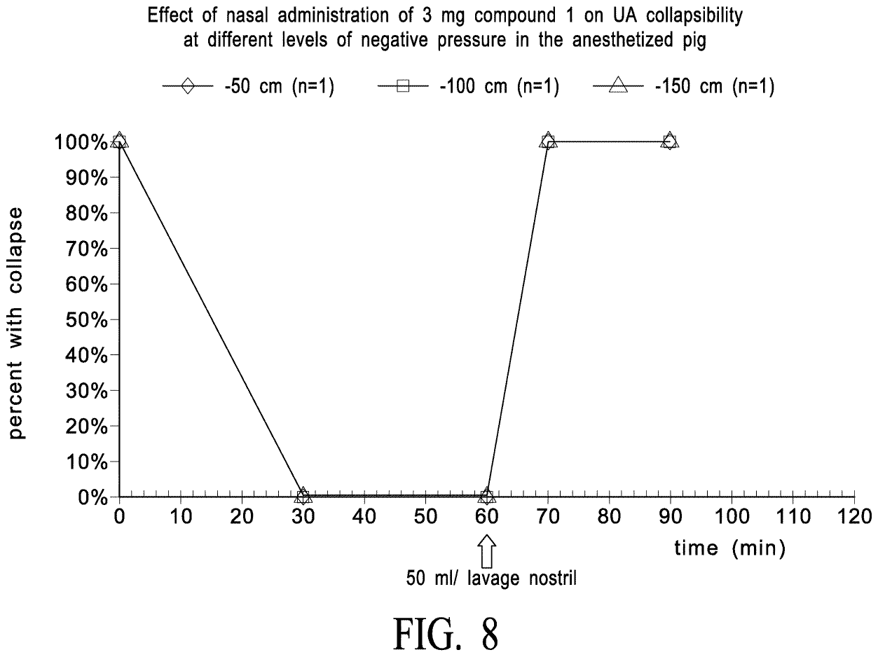

The 0.3 mg compound 1 solution formulation was administered nasally (400 μl per nostril) in one pig and the 3 mg compound 1 solution formulation was administered nasally (400 μl per nostril) in a second pig. After demonstration of full efficacy of both doses at timepoints 30 and 60 min at all collapse levels in both pigs, a nasal lavage was performed with 50 ml saline/nostril. 10 and 30 min after lavage at timepoints 70 and 90 min after drug administration, the efficacy of compound 1 was fully abolished at both doses (Table 9, Table 10, FIG. 7 and FIG. 8).

TABLE 9
Percentage of pigs showing upper airway collapsibility
(0.3 mg compound 1 solution formulation)

| Negative Pressure | | | | | |
|---|---|---|---|---|---|
| (cm H₂O) (n = 1) | 0 | 30 | 60 | 70 | 90 |
| −50 | 100% | 0% | 0% | 100% | 100% |
| −100 | 100% | 0% | 0% | 100% | 100% |
| −150 | 100% | 0% | 0% | 100% | 100% |

TABLE 10
Percentage of pigs showing upper airway collapsibility
(3 mg compound 1 solution formulation)

| Negative Pressure | | | | | |
|---|---|---|---|---|---|
| (cm H₂O) (n = 1) | 0 | 30 | 60 | 70 | 90 |
| −50 | 100% | 0% | 0% | 100% | 100% |
| −100 | 100% | 0% | 0% | 100% | 100% |
| −150 | 100% | 0% | 0% | 100% | 100% |

### Suspension Formulations:

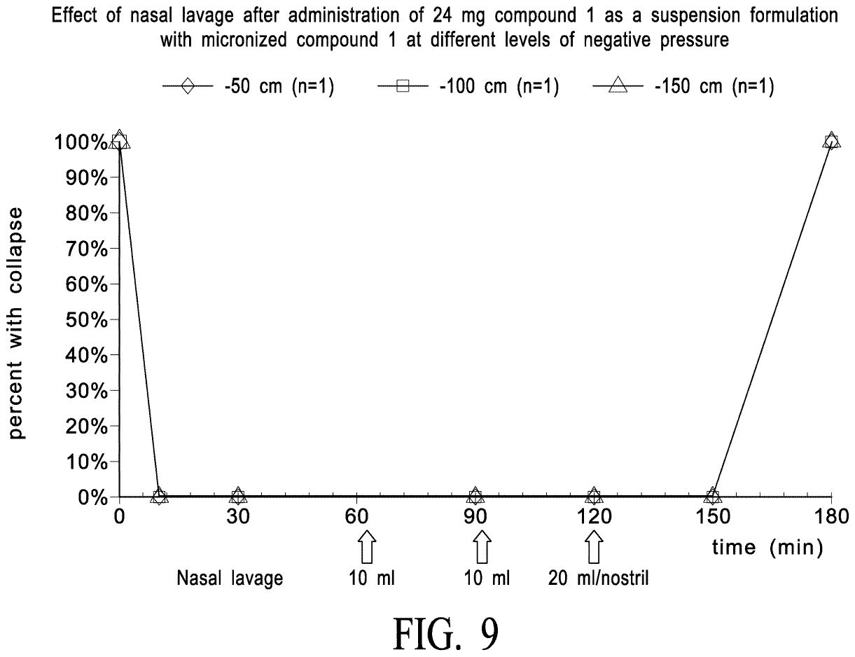

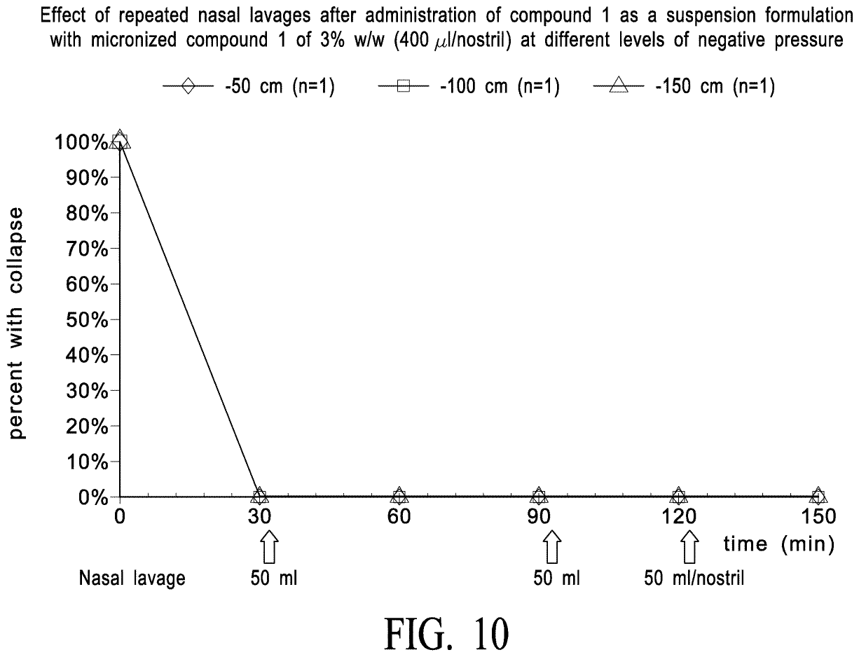

3% w/w suspension formulation: 24 mg compound 1 was administered nasally (400 μl/nostril). After demonstration of full efficacy at all collapse levels at timepoints 30 and 60 min after administrations, 60 min after administration a lavage was performed with 10 ml saline/nostril. 30 min after lavage (90 min after administration) compound 1 showed still persistent efficacy on UA collapsibility at all collapse levels. Another lavage performed after timepoint 90 after administration with 10 ml saline still showed full efficacy at timepoint 120 min. Increasing the volume of saline to 20 ml/nostril could not abolish efficacy at timepoint 150 min after administration, respectively (Table 11; FIG. 9; n=1). In second pig, 24 mg compound 1 was administered nasally (400 μl/nostril), the saline lavage volume was increased to 50 ml/nostril and the nasal lavage was performed after 30 min and repeated after 90 and 120 min after administration. Again, efficacy on UA collapsibility was maintained after nasal lavage on all collapse levels (Table 12; FIG. 10; n=1). Thus, none of the lavage regimens succeeded to abolish the upper airway collapsibility inhibition effect.

TABLE 11
Percentage of pigs showing upper airway collapsibility
(3% w/w suspension formulation)

| Negative Pressure | | | | | | |
|---|---|---|---|---|---|---|
| (cm H₂O) (n = 1) | 0 | 30 | 60 | 90 | 120 | 150 |
| −50 | 100% | 0% | 0% | 0% | 0% | 0% |
| −100 | 100% | 0% | 0% | 0% | 0% | 0% |
| −150 | 100% | 0% | 0% | 0% | 0% | 0% |

TABLE 12
Percentage of pigs showing upper airway collapsibility
(3% w/w suspension formulation)

| Negative Pressure | | | | | | |
|---|---|---|---|---|---|---|
| (cm H₂O) (n = 1) | 0 | 30 | 60 | 90 | 120 | 150 |
| −50 | 100% | 0% | 0% | 0% | 0% | 0% |
| −100 | 100% | 0% | 0% | 0% | 0% | 0% |
| −150 | 100% | 0% | 0% | 0% | 0% | 0% |

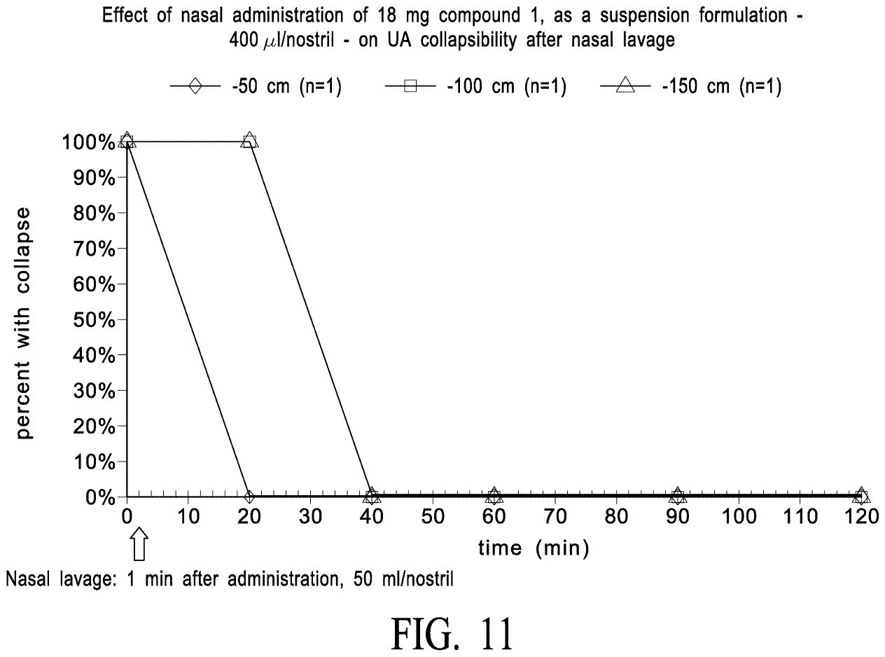

2.25% w/w suspension formulation: 18 mg compound 1 was administered nasally (400 μl per nostril) and nasal lavage was performed with 50 ml saline after only 1 min after nasal administration of 18 mg (400 μl/nostril) and prior to achieving inhibition of UA collapsibility. Also here, full inhibition of collapsibility at all pressure levels was achieved at timepoint 40 to timepoint 120 min (Table 13; FIG. 11; n=1).

TABLE 13
Percentage of pigs showing upper airway collapsibility
(2.25% w/w suspension formulation)

| Negative Pressure | | | | | | |
|---|---|---|---|---|---|---|
| (cm H₂O) (n = 1) | 0 | 20 | 40 | 60 | 90 | 120 |
| −50 | 100% | 0% | 0% | 0% | 0% | 0% |
| −100 | 100% | 100% | 0% | 0% | 0% | 0% |
| −150 | 100% | 100% | 0% | 0% | 0% | 0% |

These results demonstrate that certain suspension formulations of compound 1 are resistant to abolition of inhibition of UA collapsibility following nasal lavage. Upper airway collapsibility inhibition was maintained long after the time of lavage for the compound 1 suspension formulations at all tested doses and lavage regimens. In contrast, nasal lavage completely removed the inhibition of upper airway collapsibility by the compound 1 solution formulation at all doses and lavage regimens tested.

Therefore, failure of the phase 2 trial of Example 1 is not reasonably attributable to the use of a suspension formulation of compound 1, providing no clarity as to the reasons the phase 2 trial failed.

### 4.5.6. Example 6—Topical Intranasal Administration of Mucoadhesive Formulations of Compound 1 is Effective in Reducing Upper Airway Collapsibility in Anesthetized Pigs

### 4.5.6.1 Summary

Several nasal suspension formulations of compound 1 were investigated for ability to inhibit upper airway collapsibility in the anesthetized pig obstructive sleep apnea model in a vertical position and compared to the suspension formulation used in the failed phase 2 clinical trial (Example 1) to evaluate the in vivo performance of the formulations.

The three formulations tested at 2 mg—SU41, SU45 and SU28—were all superior to the original Ph2 formulation at 2 mg with regard to the duration and onset of action. Duration of action of the new formulations exceeded the observation period of 480 min and onset of action SU41 and SU45 was found as early as 15 min after administration, while onset of action of SU28 was at 30 and 60 min the two pigs tested. The Ph2 formulation showed onset of efficacy 30 mm after administration and had totally lost efficacy 120 m after administration.

### 4.5.6.2 Methods

Methods used are essentially as described in Example 4 with certain modifications.

### 4.5.6.3 Formulations, Preparation and Storage of Test Compounds

The novel formulations of compound 1 were provided to the test site as ready-to-use formulations, whereas the Ph2 placebo and active formulations were prepared at the test site. Compositions are set forth in Table 14, below.

TABLE 14

| | Ph2 placebo | SU40 | SU41 | SU28 | SU41 | SU45 |
|---|---|---|---|---|---|---|
| Excipient | formulation | 0.25% API | 0.25% API | 0.25% API | 9% API | 0.25% API |
| Compound 1 | — | 0.25 | 0.25 | 0.25 | 9.00 | 0.25 |
| Water | 99.31 | — | — | — | — | — |
| Citrate-phosphate | — | 96.16 | 96.91 | 96.81 | 88.41 | 95.96 |
| buffer pH 6.0 | | | | | | |
| Mannitol | 0.51 | — | — | 1.75 | — | — |
| Sorbitol | — | 2.49 | 1.75 | | 1.59 | 2.49 |
| Polyoxyl 40 | — | — | — | 0.50 | — | — |
| hydrogenated | | | | | | |
| castor oil (RH40) | | | | | | |
| Polyoxyl 35 castor | — | 0.50 | 0.50 | — | 0.46 | 0.50 |
| oil | | | | | | |
| Benzalkonium- | 0.002 | — | — | — | — | — |
| chloride | | | | | | |
| Potassium sorbate | — | 0.10 | 0.10 | 0.10 | 0.09 | 0.10 |
| EDTA | — | — | — | — | — | 0.20 |

TABLE 14

| | Ph2 placebo | SU40 | SU41 | SU28 | SU41 | SU45 |
|---|---|---|---|---|---|---|
| Excipient | formulation | 0.25% API | 0.25% API | 0.25% API | 9% API | 0.25% API |
| Microcrystalline- | 0.18 | — | — | — | — | — |
| cellulose | | | | | | |
| Methyl Cellulose | — | — | — | 0.60 | — | — |
| Carrageenan | — | 0.50 | — | — | — | 0.50 |
| (Viscarin GP 109 | | | | | | |
| NF) | | | | | | |
| Benecel E4M | — | — | 0.50 | — | 0.46 | — |
| Pharm (HPMC) | | | | | | |
| Total | 100.00 | 100.00 | 100.00 | 100.00 | 100.00 | 100.00 |

The ready-to use formulations were stored at room temperature in brown glass vials and protected from light. On the day of the respective experiment, the formulation was stirred at 1000 rpm with a magnetic stirrer (Heidolph®) for one hour, followed by sonication (HBM®) for 10 min and shaken by hand just before administration.

The Ph2 active formulation (see Table 1, Example 1 above) was freshly prepared on the respective days of the experiments: 7.5 mg micronized compound 1 drug substance was suspended in 3 ml of the diluent formulation, stirred at 1000 rpm with a magnetic stirrer (Heidolph®) for one hour, followed by sonication (HBM®) for 10 min and shaken by hand just before administration.

The suspensions were visually checked for homogeneity just before nasal administration. Then the suspension was delivered with an Eppendorf-pipette at a volume of 100, 200 or 400 μl into each nostril depending on the experiment. The vertical position of the nose allowed the test compound to flow to all parts of the pharynx.

### 4.5.6.4 Results

Novel nasal suspension formulations of compound 1 were tested in the pig model of upper airway (UA) collapsibility. These compositions are listed in Table 14 above; the comparator Ph2 formulation is listed in Table 1. All of the formulations fully inhibited UA collapsibility for a certain time period, but they differed with regard to the dose required to show full efficacy, with regard to the duration of action, and the time to onset of action.

A suspension formulation referred to as the original Ph2 formulation, and that had been used in the failed phase 2a study described in Example 1, served as the reference formulation. The novel suspension formulations were tested and compared with the Ph2 reference formulation.

Table 15 tabulates the results of duration of action and time to onset of action of the different formulations and doses tested.

TABLE 15
Negative pressure level

| | | | −50 cm | −100 cm | −150 cm |
|---|---|---|---|---|---|
| Formulation | | | H₂O | H₂O | H₂O |
| Ph2 Formulation | Pig1 | Duration | 120 | 60 | 60 |
| 2 mg | | Onset | 30 | 30 | 30 |
| (400 μl/nostril) | Pig2 | Duration | 150 | 90 | 60 |
| | | Onset | 30 | 30 | 30 |
| | Pig3 | Duration | 120 | 90 | 30 |
| | | Onset | 30 | 30 | 30 |
| | Pig4 | Duration | 120 | 120 | 30 |
| | | Onset | 30 | 30 | 30 |
| SU40 2 mg | Pig1 | Duration | 210* | 120 | 90 |
| (400 μl/nostril) | | Onset | 30 | 30 | 30 |
| (n = 4) | Pig2 | Duration | 210* | 120 | 90 |
| batch 5065/29 | | Onset | 30 | 30 | 30 |
| | Pig3 | Duration | 270 | 150 | 90 |
| | | Onset | 15 | 30 | 30 |
| | Pig4 | Duration | 270 | 150 | 90 |
| | | Onset | 15 | 30 | 30 |
| SU40 2 mg | Pig1 | Duration | 270 | 240 | 210 |
| (400 μl/nostril) ** | | Onset | 15 | 15 | 15 |
| (n = 1) | | | | | |
| Batch 5099/77A | | | | | |
| SU41 1 mg | Pig1 | Duration | 480 | 480 | 420 |
| (200 μl/nostril) | | Onset | 15 | 15 | 15 |
| (n = 1) | | | | | |
| Batch 5099/77C | | | | | |
| SU41 2 mg | Pig1 | Duration | 480 | 480 | 480 |
| (400 μl/nostril) | | Onset | 15 | 15 | 15 |
| (n = 1) | | | | | |
| Batch 5099/77C | | | | | |
| SU41 18 mg | Pig1 | Duration | 480 | 480 | 480 |
| (100 μl/nostril) | | Onset | 15 | 15 | 15 |
| (n = 1) | | | | | |
| Batch 5049/21F | | | | | |
| SU45 2 mg | Pig1 | Duration | 480 | 480 | 480 |
| (400 μl/nostril) | | Onset | 30 | 30 | 30 |
| (n = 2) | Pig2 | Duration | 480 | 480 | 480 |
| Batch 5166/17 | | Onset | 15 | 15 | 15 |
| SU28 2 mg | Pig1 | Duration | 480 | 480 | 480 |
| (400 μl/nostril) | | Onset | 30 | 30 | 30 |
| (n = 2) | Pig2 | Duration | 480 | 480 | 480 |
| Batch 5099/95 | | Onset | 30 | 60 | 60 |

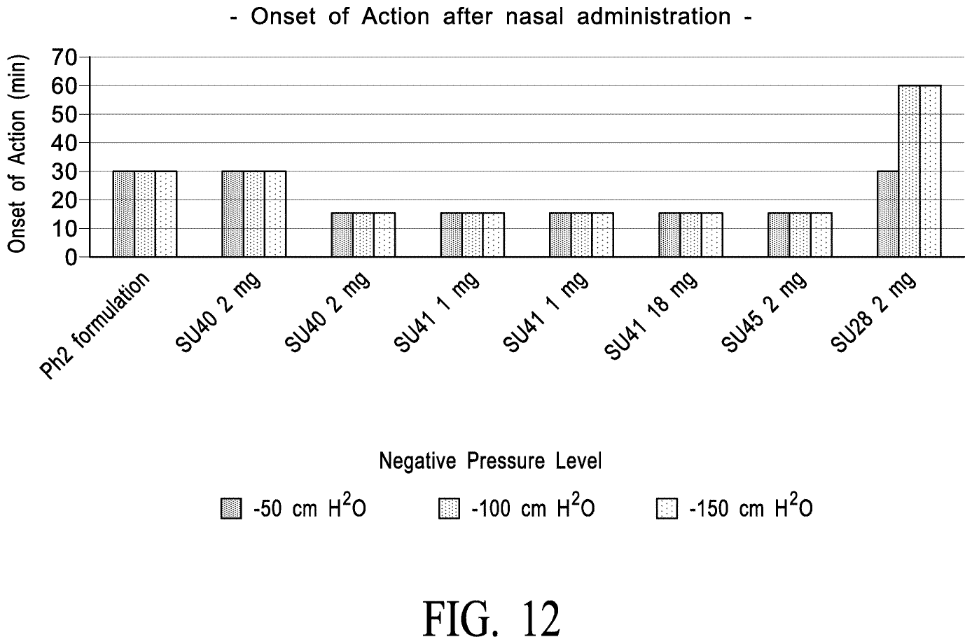

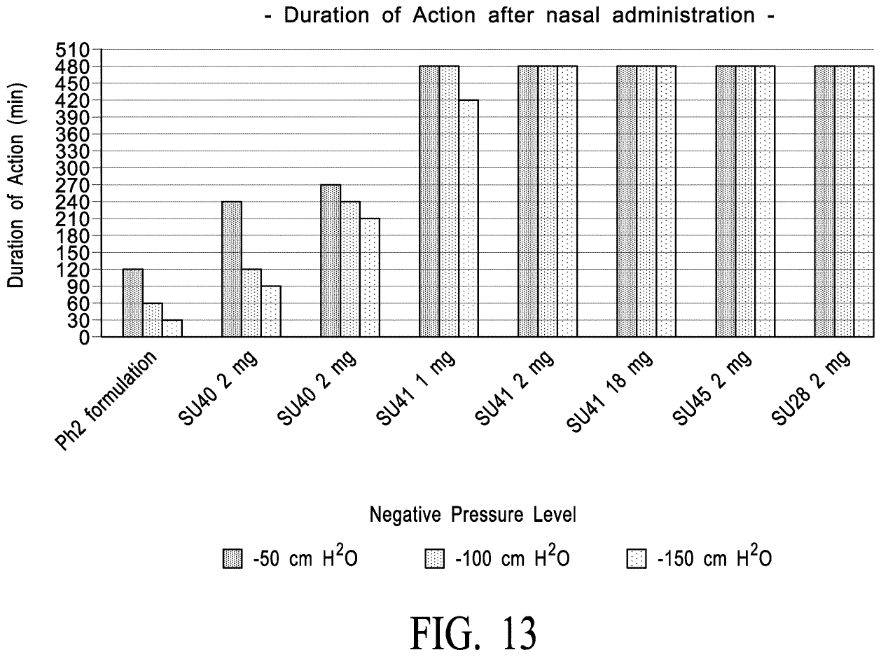

FIG. 12 shows the average time to onset-of-action for each of the formulations at each of three tested negative pressure levels: −50 cm, −100 cm, and −150 cm H₂O. FIG. 13 shows the average duration of action for each of the formulations at each of three tested negative pressure levels: −50 cm, −100 cm, and −150 cm H₂O. Observations ended at 480 minutes (8.0 hours).

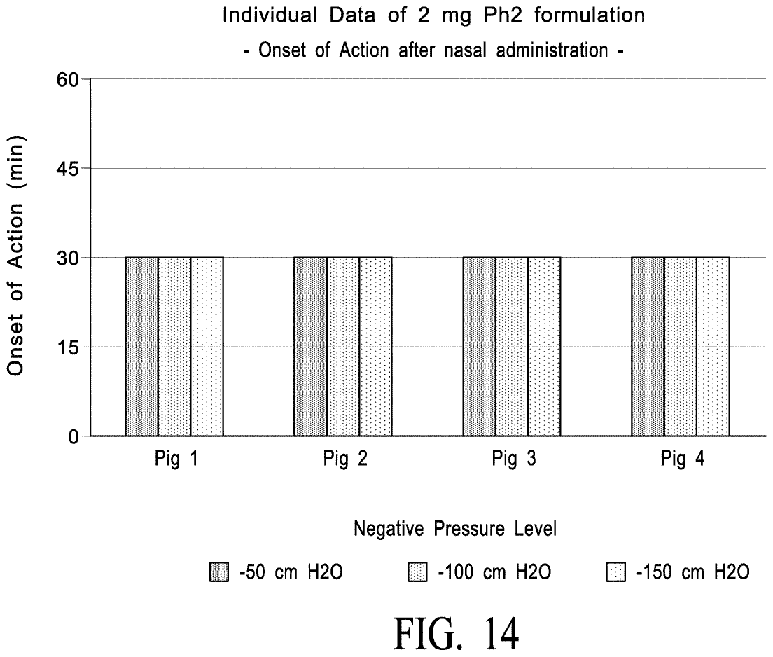

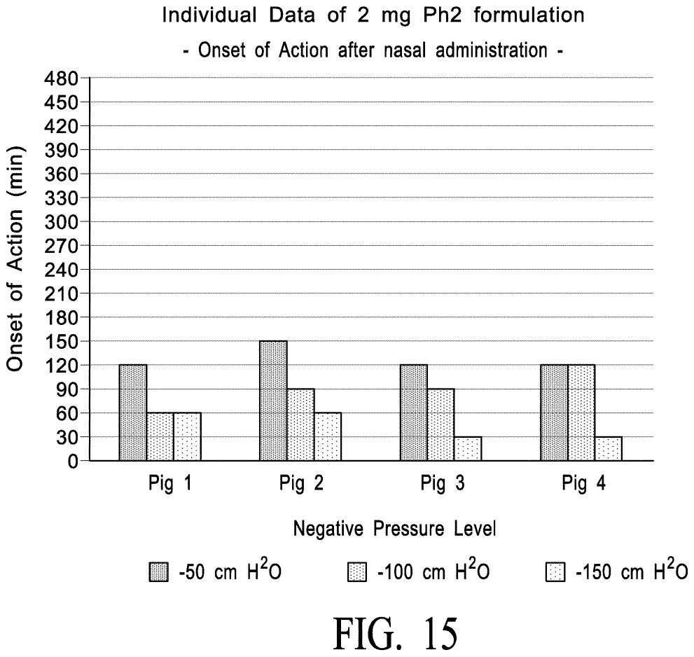

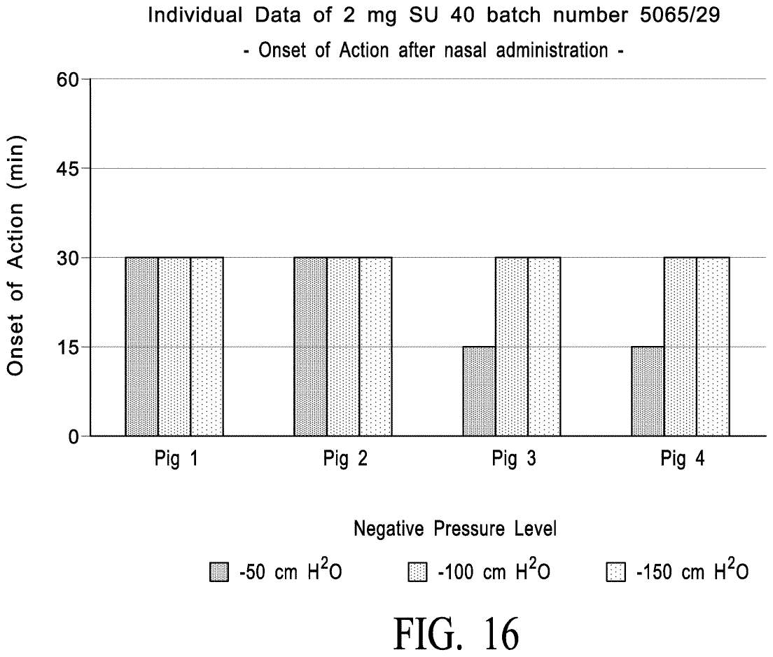

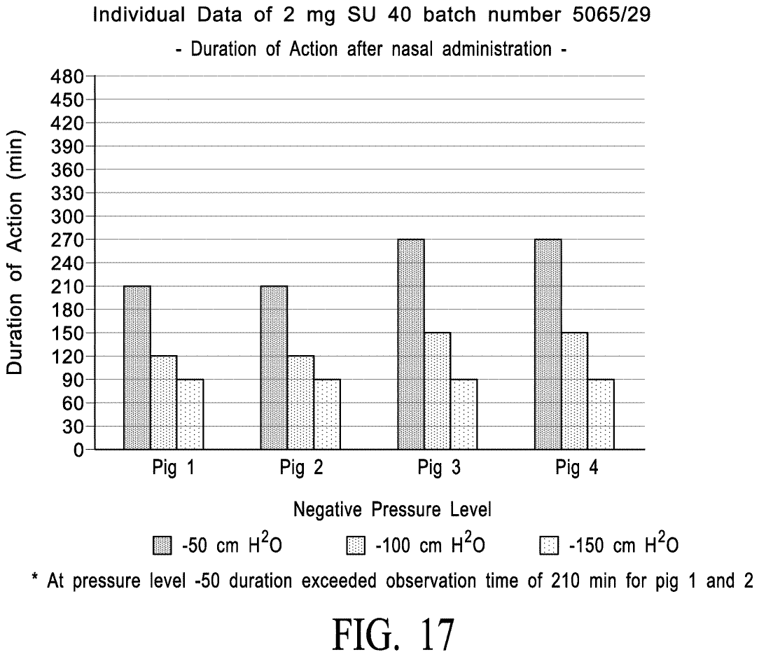

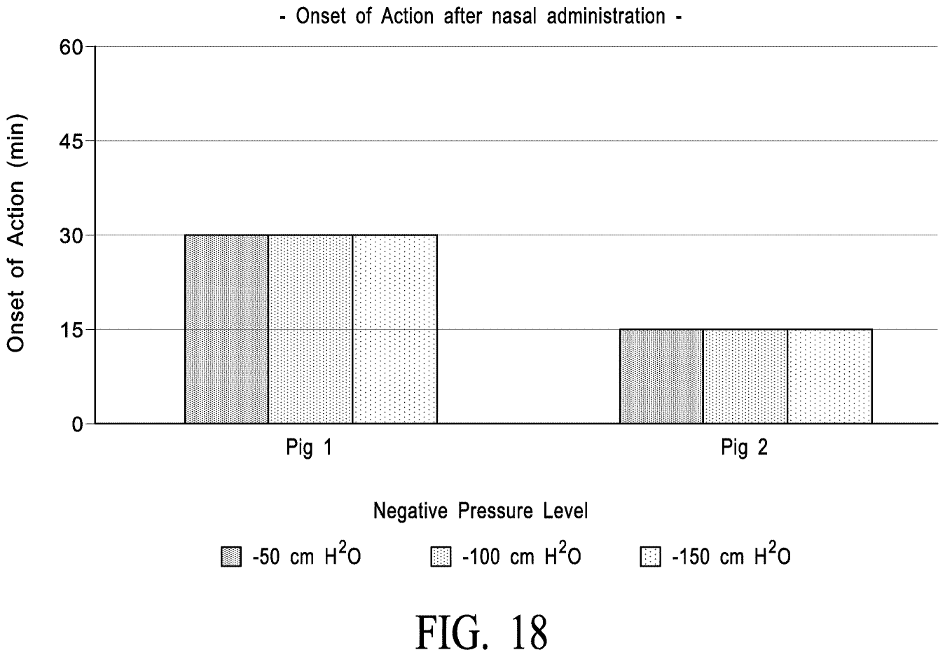

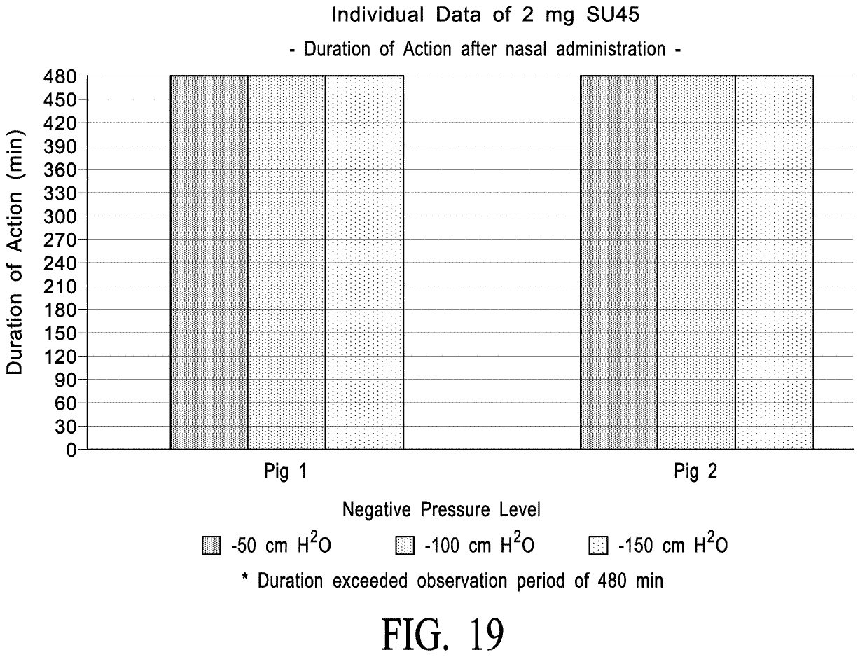

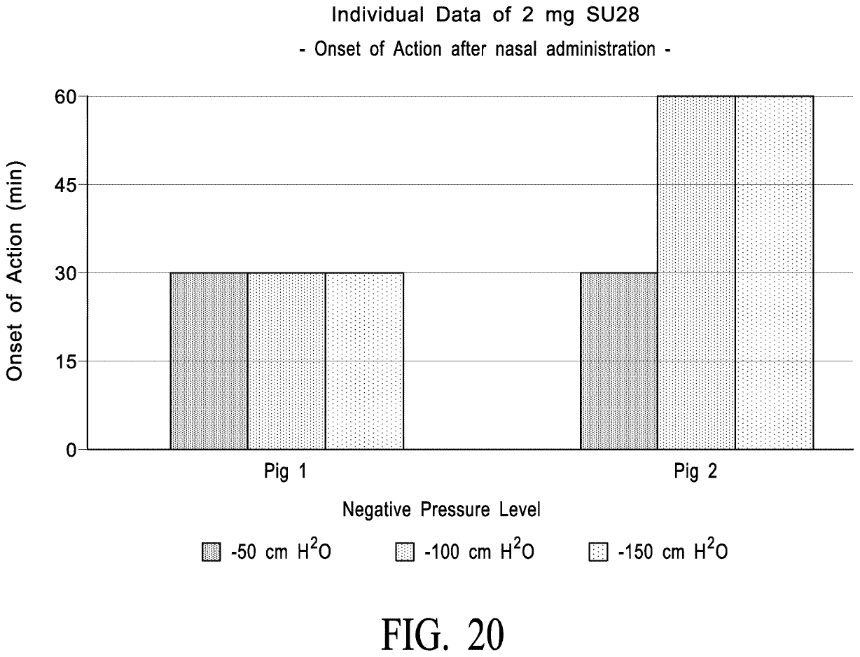

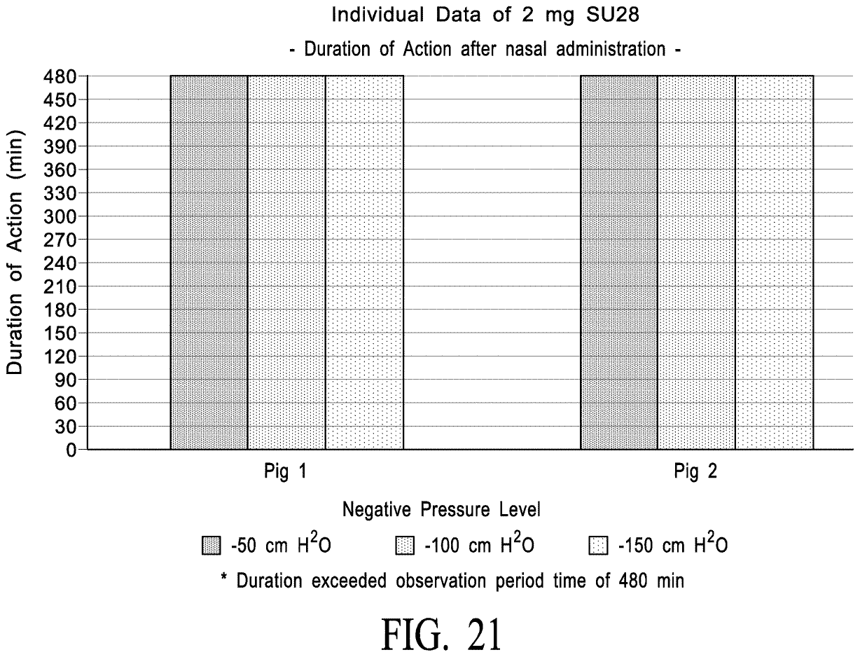

FIGS. 14-21 show data for individual pigs.

A particularly high level of efficacy was noted for the SU41 (n=1) and SU45 (n=2) at 2 mg suspension formulations, administered with 400 μl per nostril, at the same dose as the original Ph2 formulation. Duration of action exceeded the observation period of 480 min at all pressure levels tested. Onset of action occurred at the first timepoint of the measurement, at 15 min after administration of the formulation.

SU41 was also tested at 1 mg with 200 μl per nostril (n=1) and generated an only slightly less strong response compared with the 2 mg dose. Duration of action at −150 cm H₂O was 420 min, while at −50 and −100 cm H₂O duration exceeded 480 min. SU41 formulated at 9% (w/w) was tested at a dose of 18 mg, equal to the clinical dose used for the reference Ph2 formulation in the failed phase 2 trial in Example 1, by administering 100 μl per nostril, and showed full inhibition at all measured timepoints and was indistinguishable from the 2 mg dose.

The SU28 formulation was tested at a dose of 2 mg (n=2), administered with 400 μl per nostril. This resulted in a duration of action that also exceeded the 480 min observation period at all pressure levels. However, the onset of action was delayed versus the SU41 and SU45 formulations, and was 30, 30, and 30 min, respectively, at −50, −100 and −150 cm H20 respectively.

The SU40 formulation was tested at 2 mg, administered in 400 μl per nostril (n=4 pigs), and showed a full inhibition of collapsibility but with a reduced duration of action at −50, −100 and −150 cm H₂O. For the SU40 formulation, the impact of particle size distribution was evaluated by preparing an additional SU40 formulation (batch 5099/77A), with drug substance obtained after sieving non micronized batch compound 1 with a 32 μm mesh (which gave a D₉₀ of approximately 28 μm) instead of micronized drug substance (D₉₀ of approximately 4.9 μm), which was used in all other novel formulations and the Ph2 reference formulation. The alternate SU40 formulation (batch 5099/77A) was tested at 2 mg, administered in 400 μl per nostril (n=1 pig), and showed a full inhibition of collapsibility but with an increased duration of action at −50, −100 and −150 cm H₂O of 270, 240 and 210 min, respectively, and a reduced onset of action of 15 minutes, compared with the SU40 formulation obtained from micronized drug substance.

In conclusion, three novel mucoadhesive suspension formulations tested at 2 mg—SU41, SU45 and SU28—were strongly superior to the original Ph2 suspension formulation at 2 mg which had been used in the failed phase 2a OSA patient study (Example 1) with respect to duration and onset of action. Duration of action of the new suspension formulations (SU45, SU41 and SU28) exceeded the observation period of 480 min and onset of action was found at either 15 minutes or later (for SU41 and SU45) or at 30 minutes or later (for SU28) after administration. The experimental data obtained for the two SU40 formulations, obtained from either micronized or sieved drug substance, indicate that the particle size distribution of the suspended drug substance might play a role in the duration of action of the formulation.

These formulations, administered intranasally, will be effective in reducing the severity of obstructive sleep apnea in human patients.

### 4.5.7. Example 7—Properties of Formulations Containing Compound 1

SU28 and SU40 formulations were assessed during a short-term stability program with 25° C. and 40° C. storage conditions at 2 and 4 weeks. Formulations were assessed in Aptar VP7 pumps with glass bottles and AeroPump pumps with polyethylene bottles. The following tests were performed:
Compound 1 content in total and content in solution
Compound 1 related substances
Macroscopic appearance
Microscopic appearance
Apparent
Osmolality
Particle size by laser diffraction
Droplet size by laser diffraction
NGI data
Raman assessment

### 4.5.7.1 Compound 1 Recovery and Related Substances

The content (total content and content in solution) and purity (% area) of compound 1 were assessed at t=0 and following 4 weeks of storage at 25° C. and 40° C. The analytical method for determining compound 1 is provided in Table 16. The results are presented in Table 17 (total recovery), Table 18 (purity) and Table 19 (content in solution).

TABLE 16
HPLC Method

| Column | Waters Symmetry C18, 150 × 4.6 mm, | | |
|---|---|---|---|
| | 3.5 μm | | |
| Guard Column | Waters Symmetry C18 VanGuard Cartridge, | | |
| | 5 × 3.9 mm ID, 5 μm, 100 Å or equivalent | | |
| Mobile phase A | 0.1% v/v TFA in 90:10 water:acetonitrile | | |
| Mobile phase B | 0.075% TFA in 90:10 acetonitrile:water | | |
| Initial flow rate | 1.0 mL/min | | |
| Run time | 14 minutes | | |
| Wavelength | 262 nm | | |
| Column | 30° C. | | |
| temperature | | | |
| Autosampler | 20° C. | | |
| temperature | | | |
| | Time | % Mobile | % Mobile |
| Flow gradient | (min) | Phase A | Phase B |
| | 0.00 | 90 | 10 |
| | 7.00 | 40 | 60 |
| | 8.00 | 0 | 100 |
| | 11.00 | 0 | 100 |
| | 11.01 | 90 | 10 |
| | 14.00 | 90 | 10 |

| | |
|---|---|
| Injection volume | 10 μL |
| API retention time | 5.4 mins |
| Diluent (Standard | 0.1% v/v TFA in Water |
| and Sample) | |
| Needle Wash | 0.1% v/v trifluoroacetic acid in 60:40 |
| | methanol:water |
| Seal wash | 90:10 v/v water:isopropyl alcohol |
| Line storage | 60:40 v/v methanol:water |

TABLE 17
Percentage recovery (total content) of compound 1 in formulations stored in various packaging types at t =
0 and following storage for up to 4 weeks at 25° C. and 40° C. (mean of n = 3 replicates, range in brackets).

| | | Compound 1 recovery from total content (%) of formulations at | | | | |
|---|---|---|---|---|---|---|
| | | t = 0 and following storage for up to 4 weeks at 25° C. and 40° C. | | | | |
| Packaging | | | 2 weeks | | 4 weeks | |
| Type | Formulation | t = 0 | 25° C. | 40° C. | 25° C. | 40° C. |
| | SU28 6% ACT | 105.76 | 106.58 | 104.87 | 105.46 | 102.69 |
| | | (104.61-107.8) | (104.04-116.16) | (103.58-107.39) | (104.44-106.26) | (101.63-103.89) |
| | SU28 9% ACT | 103.76 | 103.85 | 105.68 | 104.07 | 103.48 |
| | | (102.42-105.29) | (101.15-107.75) | (102.87-107.88) | (103.00-105.72) | (102.72-104.39) |
| | SU41 9% ACT | 110.08 | 105.90 | 107.19 | 101.88 | 102.53 |
| | | (109.15-111.82) | (100.80-111.11) | (101.80-116.08) | (99.36-106.64) | (102.06-103.40) |
| Non-Sterile | SU28 6% ACT | 103.35 | 100.42 | 108.11 | 110.94 | 106.75 |
| Glass | | (102.51-104.78) | (100.24-100.52) | (107.44-108.47) | (109.81-111.56) | (106.15-107.19) |
| Packaging | SU28 9% ACT | 93.27 | 100.35 | 102.82 | 103.21 | 104.34 |
| | | (92.73-94.31) | (98.07-101.51) | (101.26-104.26) | (102.75-103.88) | (103.31-104.99) |
| | SU41 9% ACT | 100.61 | 102.39 | 110.59 | 101.62 | 105.52 |
| | | (100.46-100.84) | (101.18-103.57) | (107.01-115.67) | (100.84-102.87) | (105.32-105.78) |

TABLE 18
Percentage recovery (total content) of compound 1 in formulations stored
in various packaging types at t = 0 and following storage for up to
4 weeks at 25° C. and 40° C. (mean of n = 3 replicates, range in brackets).

| | | Compound 1 purity (%) of formulations at t = 0 | | | | |
|---|---|---|---|---|---|---|
| | | and following storage for up to 4 weeks at 25° C. and 40° C. | | | | |
| Packaging | | | 2 weeks | | 4 weeks | |
| Type | Formulation | t = 0 | 25° C. | 40° C. | 25° C. | 40° C. |
| Non-Sterile | SU28 6% ACT | 99.63 | 99.63 | 99.65 | 99.57 | 99.58 |
| Plastic | SU28 9% ACT | 99.52 | 99.62 | 99.59 | 99.56 | 99.60 |
| Packaging | SU41 9% ACT | 99.58 | 99.64 | 99.58 | 99.57 | 99.58 |
| Non-Sterile | SU28 6% ACT | 99.63 | 99.67 | 99.61 | 99.57 | 99.57 |
| Glass | SU28 9% ACT | 99.63 | 99.64 | 99.58 | 99.59 | 99.57 |
| Packaging | SU41 9% ACT | 99.59 | 99.63 | 99.63 | 99.60 | 99.61 |

TABLE 19
Recovery from solution (% w/w) of compound 1 in formulations stored in various packaging types at t =
0 and following storage for up to 4 weeks at 25° C. and 40° C. (mean of n = 3 replicates, range in brackets).

| | | Compound 1 recovery from solution (% w/w) of formulations at t = 0 | | | | |
|---|---|---|---|---|---|---|
| | | and following storage for up to 4 weeks at 25° C. and 40° C. | | | | |
| Packaging | | | 2 weeks | | 4 weeks | |
| Type | Formulation | t = 0 | 25° C. | 40° C. | 25° C. | 40° C. |
| Non-Sterile | SU28 6% ACT | 0.011 | 0.013 | 0.011 | 0.010 | 0.010 |
| Plastic | | (0.009-0.012) | (0.010-0.017) | (0.010-0.011) | (0.010-0.010) | (0.010-0.010) |
| Packaging | SU28 9% ACT | 0.011 | 0.016 | 0.012 | 0.011 | 0.009 |
| | | (0.011-0.011) | (0.012-0.024) | (0.012-0.013) | (0.010-0.014) | (0.009-0.010) |
| | SU41 9% ACT | 0.043 | 0.035 | 0.042 | 0.010 | 0.019 |
| | | (0.040-0.044) | (0.033-0.036) | (0.037-0.046) | (0.010-0.010) | (0.019-0.019) |
| Non-Sterile | SU28 6% ACT | 0.009 | 0.010 | 0.010 | 0.009 | 0.009 |
| Glass | | (0.008-0.011) | (0.009-0.010) | (0.010-0.010) | (0.008-0.009) | (0.008-0.010) |
| Packaging | SU28 9% ACT | 0.010 | 0.014 | 0.010 | 0.009 | 0.010 |
| | | (0.009-0.012) | (0.012-0.016) | (0.010-0.010) | (0.008-0.009) | (0.010-0.010) |
| | SU41 9% ACT | 0.021 | 0.030 | 0.042 | 0.009 | 0.010 |
| | | (0.019-0.023) | (0.029-0.031) | (0.042-0.042) | (0.008-0.009) | (0.009-0.010) |

### 4.5.7.2 Macroscopic Appearance

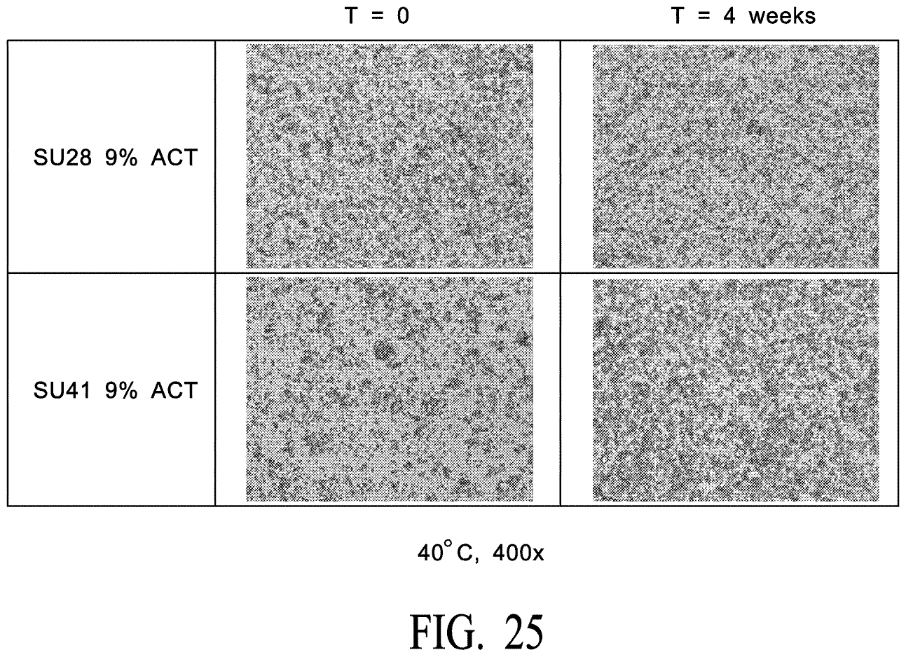

The macroscopic characteristics (i.e., color, clarity, visual viscosity) of the developed formulations was assessed at t=0 and up to 4 weeks of storage at 25° C. and 40° C. The results are presented in Table 20 and Table 21. Representative images are provided in FIG. 25. At t=0, all formulations were white, opaque suspensions with low viscosity.

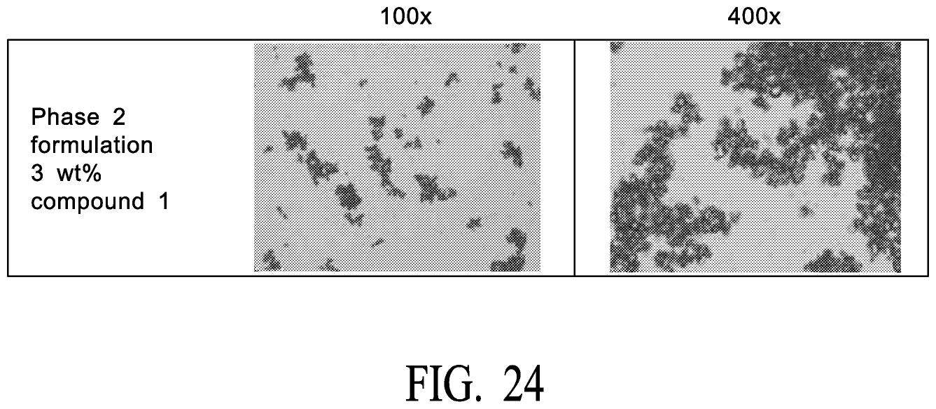

Following 4 weeks of storage at 25° C. and 40° C., there was no change in the macroscopic appearance of the formulations. Notably, the Phase 2 formulation (3 wt % compound 1) showed agglomeration at t=0 at 100× and 400× (FIG. 24).

TABLE 20
Microscopic observations of formulation stored in non-sterile
plastic packaging at t = 0 and following storage for up
to 4 weeks at 25° C. and 40° C. (under ×100 and ×400 magnification).

| | Microscopic observations of formulations at t = 0 and | | | | |
|---|---|---|---|---|---|
| | following storage for up to 4 weeks at 25° C. and 40° C. | | | | |
| Formu- | | 2 weeks | | 4 weeks | |
| lation | t = 0 | 25° C. | 40° C. | 25° C. | 40° C. |
| SU28 | Primary particles | No change | | No change | |
| 6% ACT | | from t = 0 | | from t = 0 | |
| SU28 | | | | | |
| 9% ACT | | | | | |

| | | | | |
|---|---|---|---|---|
| SU41 | Primary particles | No change | Primary | No change |
| 9% ACT | and small | from t = 0 | particles | from t = 0 |
| | agglomeration | | | |

ACT refers to wt % compound 1

TABLE 21
Microscopic observations of formulation stored in
non-sterile glass packaging at t = 0 and following
storage for up to 4 weeks at 25° C. and 40° C.

| | Microscopic observations of formulations at t = 0 and | | | | |
|---|---|---|---|---|---|
| | following storage for up to 4 weeks at 25° C. and 40° C. | | | | |
| Formu- | | 2 weeks | | 4 weeks | |
| lation | t = 0 | 25° C. | 40° C. | 25° C. | 40° C. |
| SU28 6% | Primary | No change | | No change | |
| ACT | particles | from t = 0 | | from t=0 | |
| SU28 9% | | | | | |
| ACT | | | | | |

| | | | |
|---|---|---|---|
| SU41 9% | Primary | No change | No change |
| ACT | particles | from t = 0 | from t = 0 |

### 4.5.7.3 Apparent pH

The apparent pH of the manufactured formulations was assessed at t=0 and following storage at 25° C. and 40° C. for up to 4 weeks. The results are presented in Table 22. The apparent pH of the formulations was taken following centrifugation of the samples for the content in solution extraction to give more consistent pH readings as the suspended drug interfered with the pH meter and gave more variable results. Over the 4-week study duration, the pH remained stable (less than 1 pH unit change from t=0).

TABLE 22
Apparent pH of formulation stored in various packaging
types at t = 0 and following storage for up
to 4 weeks at 25° C. and 40° C.

| | | pH observations of formulations at | | | | |
|---|---|---|---|---|---|---|
| | | t = 0 and following storage for | | | | |
| | | up to 4 weeks at 25° C. and 40° C. | | | | |
| Packaging | Formu- | | 2 weeks | | 4 weeks | |
| Type | lation | t = 0 | 25° C. | 40° C. | 25° C. | 40° C. |
| Non- | SU28 | 5.92 | 5.93 | 5.95 | 5.95 | 5.97 |
| Sterile | 6% ACT | | | | | |
| Plastic | SU28 | 5.89 | 5.91 | 5.92 | 5.99 | 5.97 |
| Packaging | 9% ACT | | | | | |
| | SU41 | 5.93 | 5.93 | 5.98 | 6.02 | 5.96 |
| | 9% ACT | | | | | |
| Non- | SU28 | 5.94 | 5.93 | 5.95 | 6.01 | 5.97 |
| Sterile | 6% ACT | | | | | |
| Glass | SU28 | 5.96 | 5.94 | 5.95 | 6.01 | 5.97 |
| Packaging | 9% ACT | | | | | |
| | SU41 | 5.96 | 5.92 | 6.00 | 6.10 | 6.04 |
| | 9% ACT | | | | | |

ACT refers to wt % compound 1

### 4.5.7.4 Osmolality

The osmolality of the manufactured formulations was assessed at t=0 and following storage at 25° C. and 40° C. for up to 4 weeks. The results are presented in Table 23.

TABLE 23
Osmolality of formulation stored in various packaging types at t = 0 and
following storage for up to 4 weeks at 25° C. and 40° C.

| | | Osmolality of formulations at t = 0 and following | | | | |
|---|---|---|---|---|---|---|
| | | storage for up to 4 weeks at 25° C. and 40° C. | | | | |
| Packaging | Formu- | | 2 weeks | | 4 weeks | |
| Type | lation | t = 0 | 25° C. | 40° C. | 25° C. | 40° C. |
| Non- | SU28 | 306 | 304 | 300 | 305 | 303 |
| Sterile | 6% ACT | | | | | |
| Plastic | SU28 | 306 | 312 | 306 | 310 | 309 |
| Packaging | 9% ACT | | | | | |
| | SU41 | 303 | 305 | 306 | 303 | 308 |
| | 9% ACT | | | | | |
| Non- | SU28 | 304 | 305 | 304 | 303 | 304 |
| Sterile | 6% ACT | | | | | |
| Glass | SU28 | 306 | 309 | 310 | 308 | 306 |
| Packaging | 9% ACT | | | | | |
| | SU41 | 305 | 303 | 307 | 304 | 305 |
| | 9% ACT | | | | | |

ACT refers to wt % compound 1

All formulations had osmolality values within the isotonic range (270 to 310 mOsm/kg). Following 4 weeks of storage at 25° C. and 40° C., no obvious change in the osmolality was observed.

### 4.5.7.5 Particle Size by Laser Diffraction

The particle size (D₉₀, μm) of the manufactured formulations was assessed at t=0 and following storage at 25° C. and 40° C. for up to 4 weeks. A SympaTEC particle sizer with Cuvette Module was used to assess the particle size of compound 1 using 0.5% Tween 20 as the dispersing medium. The results are presented in Table 24.

TABLE 24
Particle size (D₉₀, μm) of compound 1 in formulation
stored in various packaging types at t = 0 and
following storage for 4 weeks at 25° C. and 40° C.

| | | Particle size (D90, μm) in formulations in 3 | | | | |
|---|---|---|---|---|---|---|
| | | packaging types at t = 0 and following | | | | |
| | | storage for 4 weeks at 25° C. and 40° C. | | | | |
| Packaging | Formu- | | 2 weeks | | 4 weeks | |
| Type | lation | t = 0 | 25° C. | 40° C. | 25° C. | 40° C. |
| Non- | SU28 | 5.15 | 5.88 | 4.71 | 4.37 | 4.39 |
| Sterile | 6% ACT | | | | | |
| Plastic | SU28 | 4.82 | 5.76 | 4.43 | 4.01 | 5.45 |
| | 9% ACT | | | | | |
| | SU41 | 6.48 | 5.99 | 5.57 | 6.04 | 5.70 |
| | 9% ACT | | | | | |
| Non- | SU28 | 6.73 | 6.03 | 5.16 | 5.46 | 4.97 |
| Sterile | 6% ACT | | | | | |
| Glass | SU28 | 5.62 | 5.24 | 4.87 | 4.84 | 4.92 |
| | 9% ACT | | | | | |
| | SU41 | 8.79 | 7.52 | 6.91 | 6.06 | 5.90 |
| | 9% ACT | | | | | |

ACT refers to wt % compound 1

Overall, all formulations, performed well with a particle size in the expected range (5-15 μm) and did not change over time.

### 4.5.7.6 Droplet Side by Laser Diffraction

The droplet size (D₉₀, μm) of the manufactured formulations was assessed by laser diffraction using the SympaTec nasal sprayer at t=0 and following storage at 25° C. and 40° C. for up to 4 weeks. The results are presented in Table 25.

TABLE 25
Droplet size (D₉₀, μM) by laser diffraction of formulations at t = 0 and
following storage for up to 4 weeks at 25° C. and 40° C.

| | | Droplet size (D₉₀, μm) by laser diffraction of formulations at t = 0 | | | | |
|---|---|---|---|---|---|---|
| | | and following storage for up to 4 weeks at 25° C. and 40° C. | | | | |
| Packaging | | | 2 weeks | | 4 weeks | |
| Type | Formulation | t = 0 | 25° C. | 40° C. | 25° C. | 40° C. |
| Non- | SU28 | * | * | * | * | * |
| Sterile | 6% ACT | | | | | |
| Plastic | SU28 | * | * | * | * | * |
| Packaging | 9% ACT | | | | | |
| | SU41 | * | ≥608.48 ± | ≥708.83 ± | ≥698.11 ± | * |
| | 9% ACT | | 53.21 | 61.57 | 32.45 | |
| Non- | SU28 | 91.48 ± | 99.34 ± | 108.58 ± | 103.81 ± | 94.64 ± |
| Sterile | 6% ACT | 59.97 | 59.97 | 5.44 | 8.37 | 4.93 |
| Glass | SU28 | 100.07 ± | 89.61 ± | 110.12 ± | 96.02 ± | 94.62 ± |
| | 9% ACT | 2.86 | 4.85 | 83.43 | 3.92 | 3.30 |
| | SU41 | ≥706.57 ± | ≥669.78 ± | ≥606.29 ± | ≥687.39 ± | ≥660.56 ± |
| | 9% ACT | 63.64 | 46.81 | 122.00 | 37.47 | 49.85 |

ACT refers to wt % compound 1

At t=0 the droplet size (D₉₀, μm) was either close to 100 μm or >706.57 μm. These values corresponded with how the formulation visibly appeared post actuation. For formulations with a droplet size of ca. 100 μm, the actuation was “mist” like with a cone-shaped actuation. For formulations with larger droplet sizes, these formulations actuated more like a “jet” where a single stream of the formulation was observed. All “mist” formulations (e.g., SU28) contained methyl cellulose as the gelling agent whereas the “jet” formulations (e.g., SU41 and SU43) contained either carrageenan or HPMC. A wide range of droplet sizes and spray types (“mist” vs “jet) were observed when using the same packaging across the different formulations and demonstrated that formulation components impacted droplet size/spray plume geometry.

The SympaTec droplet sizing equipment configuration used was designed based on the manufacturer's recommendation for nasal products, however, for the “jet” like actuation the instrument was not able to accurately capture the droplet size, only a section of the normal distribution. As a result, the values for all of these formulations are not true reflections of the droplet size.

Following 4 weeks of storage there were no notable trends for a change in the droplet size. Overall, the droplet size data demonstrated that for formulations with measurable droplet size (i.e., “mist” like; SU28 (both strengths)) the droplet size was stable over the duration of the study.

### 4.5.7.7 Raman Analysis

Raman analysis of compound 1 in the developed nasal formulations was assessed at t=0 and following storage for 4 weeks at 40° C., to determine whether any change in the polymorphic form occurred over time (data not shown). This was performed as there are other known polymorphs of compound 1 (e.g., polymorph A). At t=0 and following 4 weeks of storage at 40° C., there were no observations of polymorph A, and the only form observed corresponded with the spectra for the starting material polymorph B.

### 4.5.7.8 SU45

Following the results of the formulation stability experiments, SU40 (6% w/w compound 1) appeared to agglomerate. A modified formulation with 9% (w/w) compound 1 and EDTA (0.2% w/w) was also manufactured (SU45). The compositions are detailed in Table 26.

TABLE 26
Composition (% w/w) of the SU40 vehicle and
the modified versions of SU40 containing EDTA

| Excipients | SU40 9% | SU45 9% |
|---|---|---|
| Citrate-phosphate | 96.40 | 96.20 |
| buffer pH 6.0 | | |
| Sorbitol | 2.50 | 2.50 |
| Polyoxyl 35 castor | 0.50 | 0.50 |
| oil | | |
| Potassium sorbate | 0.10 | 0.10 |
| EDTA | — | 0.2 |
| Carrageenan (Viscarin | 0.50 | 0.50 |
| GP 109 NF) | | |
| Total | 100.00 | 100.00 |

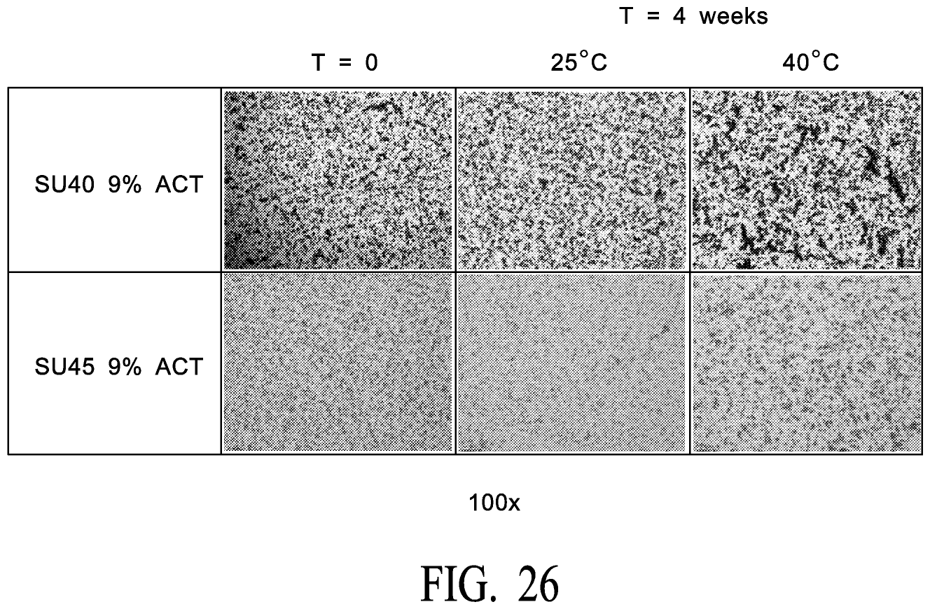

SU45 was placed on stability with storage at 25° C. and 40° C. The results of particle size by laser diffraction are shown in Table 27 and representative microscopic images at t=0 for SU40 and SU45 at 9% w/w compound 1 and following 4 weeks storage at 25 and 40° C. are shown in FIG. 26.

TABLE 27
Particle size by laser diffraction (D₉₀, μM) of the developed formulations
at t = 0 and following 4 weeks storage at 25 and 40° C.

| | | T = 2 weeks | | T = 4 weeks | |
|---|---|---|---|---|---|
| Formulation | T = 0 | 25° C. | 40° C. | 25° C. | 40° C. |
| SU45 9% API | 7.93 | 7.34 | 10.48 | 7.81 | 9.28 |
| SU40 9% API* | 7.66 | 10.19 | 7.85 | 9.77 | 9.72 |

In general, particle size appeared similar between SU40 and SU45 over the duration of the study. At t=4 weeks, the particle size data aligns with the microscopic observations whereby some agglomeration was observed at 40° C. but not at 25° C. for SU45 and agglomeration was observed at both temperatures for SU40. Microscopic images show that SU45 appears to be less agglomerated than SU40 following storage for 4 weeks at 25° C. and 40° C., although differences in particle size were only observed at 25° C. Overall, there does seem to be a reduction in agglomeration with the inclusion of EDTA in this composition.

SU45 with 9% (w/w) compound 1 was assessed in the same characterization tests run on SU28 and SU41 (Table 28)

TABLE 28
Results for SU45 (stored at 40° C. for 4 weeks followed
by ambient conditions for a further 8 weeks using) testing
according to the formulation stability assessment testing panel.

| Parameter | Result |
|---|---|
| Compound 1 recovery | 92.33 (91.30-93.56) |
| Compound 1 recovery from solution | 0.009 (0.008-0.010) |
| Compound 1 purity | 99.67 (99.64-99.69) |
| Macroscopic properties | White opaque suspension |
| Microscopic appearance | Primary particles and small |
| | agglomerates |
| Raman | Only polymorph B observed |
| Particle size D₉₀ | 9.28 μm |
| Apparent pH | 5.91 |
| Osmolality (Osmol/kg) | 351 |
| Spray Characteristics (spray | Jet like (D90 835.77 ± 253.99 μm) |
| type and droplet size)* | |

### 4.5.7.9 Viscosity

The viscosity of the three lead formulations, SU28, SU41 and SU45 was assessed. The viscosity method was developed using SU41 as the viscosity of this formulation appeared to be between SU28 (lowest viscosity) and SU45 (highest viscosity). The method was developed on a Brookfield viscometer using the S18 spindle and the small sample adaptor. Spindle 18 was selected as this spindle was capable of assessing the lowest viscosities using the instrumentation available. When developing a viscosity method, the torque value should be between 10 and 100% to get accurate readings, additionally when developing a method typically the torque should be less than 90% such that the method can still be employed if the composition thickens over time.

The developed method was using Spindle 18 at 60 RPM (which was the maximum speed of the instrument), once developed all formulations were assessed using the method and the results are presented in Table 29.

TABLE 29
Viscosity (cP) and torque (%) values for the
lead formulations (active and placebo)

Viscosity (cP) and torque (%) values for
the lead formulations (active and placebo)

| Formulation | Viscosity (cP) | Torque (%) |
|---|---|---|
| SU45 9% ACT | 45.30 | 90.60 |
| SU45 PBO | 26.30 | 52.60 |
| SU41 9% ACT | 39.20 | 78.30 |
| SU41 PBO | 23.30 | 46.60 |
| SU28 9% ACT | 4.55 | 9.10 |
| SU28 PBO | 4.75 | 9.50 |

ACT refers to wt % compound 1
PBO refers to placebo

The developed viscosity method was found to be suitable for SU41 and SU45 (torque readings between 46.6 and 90.6%), however the torque reading for SU28 was too low (ca. 9%) for an accurate reading. The viscosity of the Phase 2 formulation (3 wt % compound 1) was evaluated visually and determined to be low.

### 4.5.7.10 Sedimentation

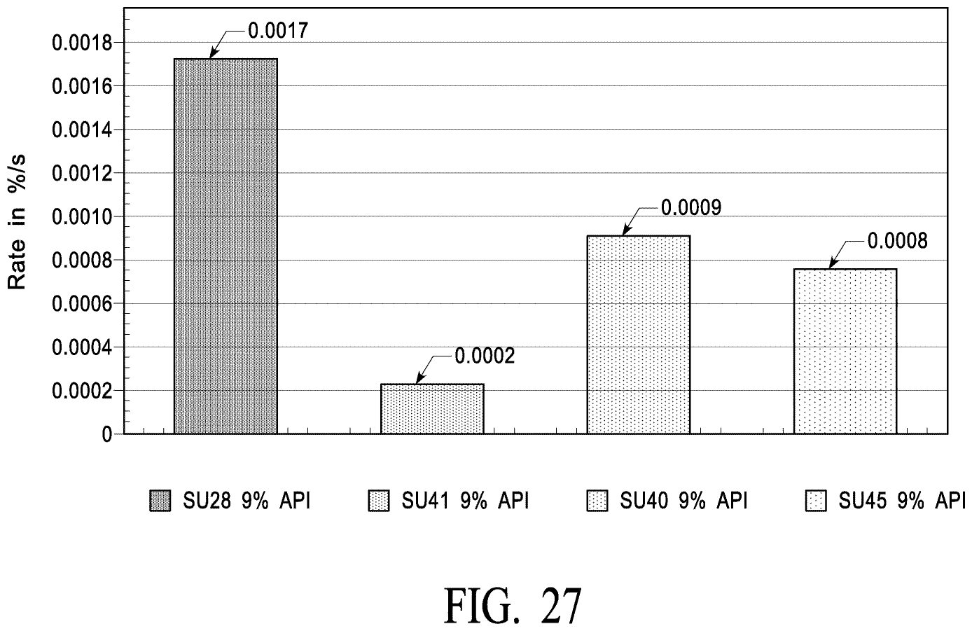

The sedimentation rate of compound 1 in the three lead formulations, SU28, SU41 and SU45, as well as SU40 (SU45 without EDTA) was assessed using a LUMiSizer. The LUMiSizer functions as a centrifuge which measures transmitted light through the sample to accurately measure when a sample begins to sediment. The 4 formulations were assessed at 200 RPM at 25° C. and the results are presented in FIG. 27. The sedimentation rate is given as a % between 0 and 1, with 1 being a sample in which sedimentation occurs quickest and 0 being a sample in which sedimentation does not occur. It should be noted that this test assesses sedimentation with a rotation force and is dependent on the speed and temperature selected. As such this test does not provide a real-world translation of sedimentation rate for the sample at rest and is typically used as a ranking tool. In this case the work was performed to provide a comparative ranking of the formulation sedimentation rates and to provide additional insight into the rheological findings. Due to the high drug loading (9% w/w) the sedimentation rate for all formulations was relatively low (due to the specific way in which the test is performed by measuring the passage of light rather than as a function of a physical law), however differences were still observed between the formulations. The sedimentation rate was highest for SU28 and lowest for SU41. SU40 and SU45 had similar sedimentation rates which suggested that the EDTA did not impact the sedimentation rate.

It was initially assumed that SU40/SU45 would have a lower sedimentation rate than SU41 due to it being the highest viscosity formulation. However, following the rheological assessment of the formulations it was determined that although SU40/45 had more polymer interactions (hence the higher viscosity), the polymer interactions in SU41 were stronger. Therefore, it is possible that the drug polymer interactions have more impact on the sedimentation rate than the viscosity.

### 4.5.7.11 Rheology

The rheological properties of the three lead formulations, SU28, SU41 and SU45, as well as SU40 (SU45 without EDTA) were assessed. All testing was performed using a TA instruments Discovery Rheometer with a double walled concentric cylinder. Formulations were prepared by first mixing in a Turbula mixer for 20 minutes with steel balls to ensure distribution of the suspended drug. Following this, approximately 11 g of formulation was added into the concentric cylinders using a 20 mL syringe.

The following rheological assessments of the formulations were performed. Due to the rheological properties of SU28 (Newtonian like fluid) only a flow sweep was performed.
Flow sweep—This test was selected to investigate the viscosity of the different formulations with increasing application of energy (using an oscillation shear rate), and to identify whether the formulations were shear-thinning (decreasing in thickness with increasing energy tangentially), shear-thickening (increasing in thickness with increasing energy tangentially) or Newtonian (the thickness does not change with changing energy). An example of a shear-thinning material/product would be ketchup, an example of a shear-thickening material would be sand in water, and an example of a Newtonian fluid is water. The viscosity (Pa·s) of the formulation sample was measured against increasing shear rate (s⁻¹).
Oscillation amplitude—This test was selected to give an indication of molecular interactions within the suspension. Additionally, the yield stress can be derived from this test which gives the stress at which the material begins to flow, i.e. how easy the formulation is to spray. The Storage and Loss moduli (Pa) were measured against increasing oscillation torque (μN·m)
Oscillation time—This test was performed to simulate the formulation at rest before being shaken, actuated, and then back to rest. The Storage and Loss moduli (Pa) of the samples were measured over time following an increase in the torque from 1 μN·m to 16 μN·m, then back down to 1 μN·m.

### Flow Sweep

The viscosity and rheological behavior of SU28 vehicle was found to match the profile of a Newtonian fluid. A Newtonian fluid (such as water) is characterized by a viscosity that is independent of the shear rate. This was identified by a linear flow sweep graphs (not shown) which was different to the other three vehicles which all show pseudoplastic (shear-thinning) behavior, i.e. viscosity decreased as shear rate increases.

SU41, SU45, and SU40 actives were all observed to be shear thinning, with significant sedimentation not observed over the duration of the test. These results aligned with other physical characterization tests performed including a sedimentation analysis (which showed SU41 to sediment the slowest, followed by SU45 with the fastest sedimentation observed for SU28. Also, the viscosity testing which confirmed that SU45/SU40 had the highest viscosity, followed by SU41 and finally SU28 with the lowest viscosity.

### Oscillation Amplitude

SU40 and SU45 demonstrated similar behavior in the oscillation amplitude with no obvious differences caused by the presence of the EDTA (not shown), which aligns with the viscosity and droplet size assessments which are similar between the two formulations. As such the remaining discussions will consider them as a single formulation.

SU40/SU45 demonstrated storage dominant behavior (i.e. more elastic/solid behavior) at low torque, whereas SU41 (not shown) was loss dominant (i.e. more viscous/liquid behavior), which aligned with the viscosity readings for the formulations. The storage dominant behavior was higher in SU40/45 than SU41 which suggested that SU40/45 had more molecular interactions than SU41.

The linear viscoelastic region (LVR) for SU40/45 was shorter than that observed for SU41 which suggested that, whilst SU40/45 had more polymer interactions than SU41 at low torque, the polymer interactions were weaker and broke more easily than the interactions observed for SU41. This should mean that SU41 requires more energy to begin to flow despite having a lower viscosity than SU40/45. It could also be that the LVR is related to the amount of agglomeration (particle size distribution), a shorter LVR is indicative of more agglomeration, though as these were freshly made no notable difference was expected.

Stronger polymer interactions (either drug polymer, or polymer polymer) may be why SU41 has been observed to sediment the slowest out of the formulations.

### Oscillation Time Sweep

As with the flow sweep and oscillation amplitude testing, SU40 and SU45 (not shown) demonstrated similar behavior in the oscillation time sweep with no obvious differences caused by the presence of the EDTA.

As with the oscillation amplitude testing, SU40/SU45 demonstrated storage dominant behavior (i.e. more elastic behavior) at low torque, whereas SU41 (not shown) was loss dominant (i.e. more viscous behavior).

SU40/45 were observed to quickly regain structure following the stress (representative of the actuation) and the storage modulus (elastic behavior) appears to increase to a value greater than before applying the stress. This potentially indicates that the stress is mixing the sample, thus better distributing the drug and causing more interactions to occur. This could mean that after spraying, the formulation quickly regains structure allowing it to be more adherent and to be less susceptible to run off.

SU41 appears to start to regain structure following the stress but then the effect of the continuous low shear further breaks down polymer interactions as indicated by the continuous drop. This could mean that after spraying SU41 the viscosity decreases, and as a result the formulation may be less likely to adhere to the tissue and be more susceptible to run off. It should, however, be noted that compound 1 sediments slowest in SU41 which is thought to be as a result of stronger interactions in this formulation (characterized by the longer LVR). As the stresses applied to SU41 during reconstitution (Turbula mixing) are much higher than those applied during the oscillation time sweep, it would appear that a stress followed by constant low shear is required to further break down polymer interactions (either polymer-polymer or polymer-drug), rather than just the stress itself.

### 4.5.7.12 Surface Tension

The surface tension of the three lead formulations, SU28, SU41 and SU45 was assessed using a Krüss Drop Shape Analyzer—DSA25E and associated Advance Software to measure the surface tension of the formulations through pendant drop methodology. The results are presented in Table 30. Surface tension was investigated because the surface tension of a spray formulation can impact how the formulation breaks up when actuated and therefore how it sprays (i.e. plume geometry/droplet size).

TABLE 30
Surface tension (mN/m) of SU28, SU41 and SU45
9% w/w compound 1 active formulations

| Sample | Surface Tension (mN/m) | Mean Surface Tension (mN/m) |
|---|---|---|
| SU28 | 44.64 | 44.89 ± 0.34 |
| | 44.75 | |
| | 45.29 | |
| SU41 | 43.47 | 43.36 ± 0.43 |
| | 43.72 | |
| | 42.89 | |
| SU45 | 43.11 | 43.39 ± 0.25 |
| | 43.51 | |
| | 43.56 | |

The results of the surface tension assessment demonstrated similar surface tensions (ca. 44 mN/m) for all three formulations.

### 4.5.8. Example 8—an Open Label Cross-Over Pharmacoscintigraphy Study Testing a Single Dose of Compound 1 Nasal Spray of 4 Different Formulations in Healthy Male and Female Volunteers

### 4.5.8.1 Summary

Technetium 99-labelled compound 1 9% (w/w) nasal suspension formulations will be evaluated in a scintigraphic imaging and pharmacokinetic study in healthy volunteers for their safety, tolerability and PK profile. The three presented nasal diluent formulations (SU28, SU41 and SU45) will be mixed at the clinical site with Technetium 99 radiolabeled drug substance by adding the diluent to the compound 1 in the device, closure of the device and subsequent mixing. The three formulations will be evaluated for their PK performance in a scintigraphy study monitoring both deposition and residence using camera images and evaluating blood pharmacokinetics. In addition, the SU45 formulation will be tested in combination with two different drug particle size distribution micronized drug substance (d90<10 μm and d90<35 μm), in order to evaluate the impact of particle size distribution on the deposition and residence time in the nose.

The radioactive formulations are manufactured on the day of use and are administered within a time frame of 12 hours.

In preparation for the clinical trial, technetium 99-labelled compound 1 9% (w/w) nasal suspension formulations using a multidose spray pump delivering doses of 100 μL were prepared and evaluated.

### 4.5.8.2 Compound 1 Batch Analysis

The following compound 1 batches were manufactured (Table 31).

TABLE 31
Overview of Compound 1 drug substance batches

| | | Batch size/ | |
|---|---|---|---|
| | Compound 1 | Manufacturing | |
| No. | Batch No. | date | Batch use |
| 1 | Batch H | 1111.48 g | GMP batch; Phase I clinical |
| | Non-micronized | 24 Jul. 2023 | trial; |
| 2 | Batch C | 863 g | non-GMP batch |
| | Non-micronized | 21 Feb. 2023 | |
| 3 | Batch E¹ | 150 g | non-GMP batch. |
| | Micronized | 28 Mar. 2023 | Tox and Technetium 99 label |
| | | | process validation |
| 4 | Batch K¹ | 45.8 g | non-GMP batch. |
| | Micronized | 10 Oct. 2023 | Tox and Technetium 99 label |
| | | | process validation batch |
| 5 | Batch L² | 164 g | GMP batch; Phase I clinical |
| | Micronized | 23 Oct. 2023 | trial; |
| 6 | Batch M² | Scheduled for | GMP batch; Phase I clinical |
| | Micronized | Q1/2024 | trial; |

¹Micronized sublots from batch C
²Micronized sublots from batch H

A summary of testing results to date is provided in Table 32.

TABLE 32
Batch analysis results

| | | Results | | | | | |
|---|---|---|---|---|---|---|---|
| | | Non-micronized | | Micronized | | | |
| | Acceptance | Cmpd 1 | Cmpd 1 | Cmpd 1 | Cmpd 1 | Cmpd 1 | Cmpd 1 |
| Test | criteria | batch H* | batch C | batch E | batch K | batch L | batch M |

CHARACTERISTICS

| | | | | | | |
|---|---|---|---|---|---|---|
| Macroscopic | White to | White | white | white | white | white |
| appearance | yellow powder | powder | solid | solid | solid | solid |

IDENTIFICATIONS

| | | | | | | |
|---|---|---|---|---|---|---|
| Infrared absorption | Complies with | Complies | / | / | / | / |
| spectrophotometry | reference spectrum | | | | | |
| ¹H and ¹³C NMR spectrum | Complies with | / | Complies | Complies | Complies | / |
| | structure | | | | | |
| X-Ray diffraction | Conform with | Complies | Conform with | Conform with | Conform with | Conform with |
| | polymorphic form B | | polymorphic | polymorphic | polymorphic | polymorphic |
| | | | form B | form B | form B | form B |
| Compound 1 | Retention time | Complies | Complies | / | | Complies |
| Identification (LC) | comparable to | | | | | |
| | reference standard | | | | | |
| | (+/−2%) | | | | | |

TESTS

| Water - micro determination | Report result | | 0.01% | | <0.1% | | / | / |
|---|---|---|---|---|---|---|---|---|
| Sulphated ash | ≤0.5% | | 0.08% | | <0.1% | | / | / |
| Elemental impurities | | | | | | | / | |
| Pd | ≤10 | ppm | <3 | ppm | / | | / | / |
| Cd | ≤30 | ppm | <9 | ppm | <9 | ppm | | / |
| Pb | ≤50 | ppm | <15 | ppm | <15 | ppm | | / |
| As | ≤20 | ppm | <6 | ppm | <6 | ppm | | / |
| Hg | ≤10 | ppm | <3 | ppm | <3 | ppm | | / |
| Co | ≤30 | ppm | <9 | ppm | <9 | ppm | | / |
| V | ≤10 | ppm | <3 | ppm | <3 | ppm | | / |
| Ni | ≤60 | ppm | <18 | ppm | <18 | ppm | | / |
| Li | ≤250 | ppm | <75 | ppm | <75 | ppm | | / |
| Sb | ≤200 | ppm | <60 | ppm | <60 | ppm | | / |
| Ba | ≤3000 | ppm | <900 | ppm | <900 | ppm | | / |
| Mo | ≤100 | ppm | <30 | ppm | <30 | ppm | | / |
| Cu | ≤300 | ppm | <90 | ppm | <90 | ppm | | / |
| Sn | ≤600 | ppm | <180 | ppm | <180 | ppm | | / |
| Cr | ≤30 | ppm | <9 | ppm | <9 | ppm | | / |

| Residual solvents | | | | | | | | |
|---|---|---|---|---|---|---|---|---|
| Methanol | ≤3000 | ppm | Not detected | | / | | | / |
| Ethanol | ≤5000 | ppm | 16 | ppm | <0.05% | | | / |
| Ethyl Acetate | ≤5000 | ppm | Not detected | | / | | | / |
| N-Butyl Acetate | ≤5000 | ppm | 333 | ppm | 0.07% | | | / |
| Toluene | ≤890 | ppm | 6 | ppm | / | | | / |
| Dichloromethane | ≤600 | ppm | Not detected | | / | | | / |
| Total residual solvents | Report result | | 355 | ppm | / | | | / |
| Related substances | | | | | | | | |
| Unspecified | ≤0.30% | | RRT | | RRT | | | RRT |
| impurities | | | 1.31 = 0.05% | | 1.09 = 0.07% | | | 1.32 = 0.06% |
| Total impurities | ≤2.5% | | 0.05% | | <0.1% | | | 0.06% |
| Total Aerobic Microbial | ≤10³ | CFU/g | <10 | CFU/g | / | / | / | / |
| Count (TACM) | | | | | | | | |
| Total Yeasts and Moulds | ≤10² | CFU/g | <10 | CFU/g | / | / | / | / |

| Count (TYMC) | | | | | | | | |
|---|---|---|---|---|---|---|---|---|
| Melting point | Report result | 115.9° | C. | 113.9° | C. | 114.2° C. | 114.0° C. | / |

| | ASSAY | | | | | | |
|---|---|---|---|---|---|---|---|
| Compound 1 ASSAY | 97.0-102.0% | 100% | 113.9° | C. | 99.9%** | 99.9%** | 101.7% |

| | | | | | |
|---|---|---|---|---|---|
| Particle size distribution | / | / | d10 0.96 μm | d10 1.81 μm | d10 0.80 μm |
| | (unmilled) | (unmilled) | d50 4.14 μm | d50 12.53 μm | d50 2.3 μm |
| | | | d90 8.85 μm | d90 30.01 μm | d90 5.9 μm |

*Micronized sublots will be prepared from batch H for use in phase 1a clinical trial.
**Chromatographic purity expressed in area %

Compound 1 identification: High performance liquid chromatography (HPLC) method in line with Ph. Eur. 2.2.29 was used to identify compound 1 drug substance and determine its assay and related substances (Table 33, Table 34, and Table 35). This test was carried out using gradient elution. The appropriate quantity of compound 1 drug substance was previously dissolved in the dissolution medium. The sample solution was then injected into a Waters Symmetry C18 column (or equivalent) and eluted with a mixture of purified water and acetonitrile containing each 0.100 of trifluoroacetic acid respectively. The working wavelength was set at 262 nm.

TABLE 33
Chromatographic conditions

| Column | Waters Symmetry C18 column | |
|---|---|---|
| | (150*3.0 mm, 5.0 μm) or equivalent | |
| Flow rate | 0.8 | mL/min |
| Injection volume | 7.0 | μL |
| Column temperature | 35° | C. |
| Sample temperature | 20° | C. |
| Analysis time | 40.0 | min |

| | |
|---|---|
| Detection UV | at 262 nm |

TABLE 34
Mobile phase composition

| | Water | Acetonitrile | TFA |
|---|---|---|---|
| Mobile phase A | 900 | 100 | 1.0 |
| Mobile phase B | 100 | 900 | 1.0 |
| Dissolution medium | 1000 | — | 1.0 |

TABLE 35
Gradient composition

| Time t₀ (min) | Mobile phase A (%) | Mobile phase B (%) |
|---|---|---|
| 0.00 | 90.0 | 10.0 |
| 3.00 | 90.0 | 10.0 |
| 15.00 | 80.0 | 20.0 |
| 17.00 | 80.0 | 20.0 |
| 22.00 | 62.0 | 38.0 |
| 29.00 | 0.0 | 100.0 |
| 30.00 | 0.0 | 100.0 |
| 31.00 | 90.0 | 10.0 |
| 40.00 | 90.0 | 10.0 |

Identification of compound 1 drug substance was performed by comparing the retention time and the UV spectrum of the main peak in the sample solution with the retention time of compound 1 in the standard solution. The retention time of the compound 1 main peak is between 14 and 16 min; UV maxima are about 205 nm and 262 nm.

Compound 1 drug substance was quantified by external calibration with standard solution at 100% of the working concentration. The compound 1 assay was calculated using following equation:

$$Compound 1 ( in \% ) = [ ( {A}_{sample} \times {DF}_{sample} ) / ( {RF}_{mean} \times {Xs}_{ample} ) ] \times 100$$

A_{Sample}—compound 1 area in the sample solution (AU)
DF_{Sample}—Dilution factor of the sample solution (L)
X_{Sample}—Sample weight (mg)
RFₘₑₐₙ—Mean of the response factors (RF) calculated on the first 6 STD-1 injections (AU*L/mg)

Related substances: Impurities were quantified by external calibration with standard solution at 1.0% of the working concentration. The impurities are calculated using following equation:

$$Impurity content ( in \% ) = [ ( {A}_{sample} \times {DF}_{sample} ) / ( {RRF}_{mean} \times {X}_{sample} ) ] \times 100$$

A_{Sample}—Impurity area in the sample solution (AU)
DF_{Sample}—Dilution factor of the sample solution (L)
X_{Sample}—Sample weight (mg)
RRF—Relative Response factor of the impurity
RFₘₑₐₙ—Mean of the response factors (RF) calculated on the first 6 STD-1 injections (AU*L/mg)

Response factor is calculated according to equation (applies for both assay and related substances):

$$RF [ AU \times ( L / mg ) = ( {A}_{standard} / {Conc}_{standard} )$$

A_{Standard}—Compound area in the standard solution (AU)
Conc_{standard}—Compound concentration in the standard solution (mg/L)

Container Closure System: Compound 1 was packaged in double low-density polyethylene (LDPE) bags and placed in high-density polyethylene (HDPE) drum.

### 4.5.8.3 Compound 1 Stability Analysis

A stability study for compound 1 batch H (non-micronized, GMP) was initiated as per ICH storage conditions described in Table 36.

Results for 1 month of storage at 25±2° C./60±5% RH, 30±2° C./65±5% RH and at 40±2° C./75±5% RH are available to date.

A stability study for compound 1 batch L and M (micronized to d90<10 μm and d90<35 μm respectively, scheduled GMP sublots of compound 1 batch H) will be initiated as per ICH reference storage conditions described in Table 37 and Table 38.

In addition, compound 1 drug substance supportive stability batch data from previous drug substance campaigns was reviewed to assess drug substance's stability.

TABLE 36
Compound 1 drug substance stability batches

| | Container closure | Storage | Testing |
|---|---|---|---|
| Batch | system | condition | frequency, months |
| A | Forced degradation study under acidic, alkaline, oxidative, | | |
| | elevated heat and humidity and light conditions. | | |
| H | Double LDPE bags | 25 ± 2° C./ | 0, 1, 3, 6, 9, 12, |
| (non- | | 60 ± 5% RH | 18, 24, 36 |
| micronized) | Double LDPE bags | 30 ± 2° C./ | 0, 1, 3, 6, 12 |
| | | 65 ± 5% RH | |
| | Double LDPE bags | 40 ± 2° C./ | 0, 1, 3, 6 |
| | | 75 ± 5% RH | |
| L | Double LDPE bags | 25 ± 2° C./ | 0, 6, 12 |
| (micronized) | | 60 ± 5% RH | |
| M | Double LDPE bags | 25 ± 2° C./ | 0, 6, 12 |
| (micronized) | | 60 ± 5% RH | |
| 4B002 | Double LDPE bags | 25 ± 2° C./ | 0, 3, 6, 12 |
| (micronized) | | 60 ± 5% RH | |
| | Double LDPE bags | 40 ± 2° C./ | 0, 3, 6 |
| | | 75 ± 5% RH | |
| D006 | Double LDPE bags | 25 ± 2° C./ | 0, 6, 18, 24, 36 |
| (non- | | 60 ± 5% RH | |
| micronized) | Double LDPE bags | 40 ± 2° C./ | 0, 3, 6 |
| | | 75 ± 5% RH | |

TABLE 37
Stability protocol for non-micronized compound 1 batch H

| | Testing frequency, months | | | | | | | | |
|---|---|---|---|---|---|---|---|---|---|
| Storage Condition | 0 | 1 | 3 | 6 | 9 | 12 | 18 | 24 | 36 |
| 25 ± 2° C./60 ± 5% RH | A + B | A | A | A | A | A + B | A | A + B | A + B |
| 30 ± 2° C./65 ± 5% RH | | A | A | A | A | A + B | | | |
| 40 ± 2° C./75 ± 5% RH | | A | A | A + B | | | | | |

A = macroscopic appearance, identification by HPLC performed only at T0, X-ray diffraction, water - micro determination, assay, related substances by HPLC; B = microbiological contamination (TAMC, TYMC).

TABLE 38
Stability protocol for micronized compound 1 batches L and M

| | Testing frequency, months | | | | | | | | |
|---|---|---|---|---|---|---|---|---|---|
| Storage Condition | 0 | 1 | 3 | 6 | 9 | 12 | 18 | 24 | 36 |

| | | | |
|---|---|---|---|
| 25 ± 2° C./60 ± 5% RH | A | A | A |

A = macroscopic appearance, identification, assay and related substances by HPLC, particle size distribution, X-ray diffraction.

To date, only limited data are available on compound 1 drug substance both non-micronized and micronized. Stability data of previous lots (manufactured in 2004) of compound 1 non-micronized polymorphic form B material demonstrated stability at both at long term conditions 25±2° C./60±5% for up to 36 months and accelerated conditions 40±2° C. /75±5% RH for up to 6 months (data not shown). The stability data of compound 1 micronized polymorphic form B material (from the same 2004 lot) demonstrated stability at both at long term conditions 25±2° C./60±5% for up to 12 months and accelerated conditions 40±2° C./75±5% RH for up to 6 months (data not shown).

### 4.5.8.4 Diluent

Diluent is used as a vehicle for the extemporaneous reconstitution of finished product (nasal suspension) formulation. For the phase 1 scintigraphy study three different diluent formulations will be used: SU28, SU41 and SU45 to reconstitute three nasal spray suspensions at 9% (w/w) compound 1 (Table 39). Each diluent formulation is a clear, colorless, and homogeneous solution free from particles. Diluents are filled in a clear borosilicate glass bottles and closed with screw cap.

TABLE 39
Qualitative and quantitative compositions of diluents

| | | | Quantity (% w/w) | | |
|---|---|---|---|---|---|
| Component | Function | Reference | SU28 | SU41 | SU45 |
| Citrate-phosphate buffer pH 6.0* | Vehicle/buffering agent | USP, Ph. | 97.05 | 97.15 | 96.20 |
| | | Eur. | | | |
| Sorbitol | Tonicity modifier | Ph. Eur. | | 1.75 | 2.50 |
| Manitol | | Ph. Eur. | 1.75 | | |
| Polyoxy 35 castor oil | Wetting agent/Surfactant | USP | | 0.50 | 0.50 |
| Polyoxy 40 hydrogenated castor oil | | Ph. Eur. | 0.50 | | |
| (RH 40) | | | | | |
| Ethylenediaminetetraacetic acid | Chelating agent | USP | | | 0.20 |
| (EDTA) | | | | | |
| Potassium sorbate | Antimicrobial preservative | Ph. Eur. | 0.10 | 0.10 | 0.10 |
| Carrageenan (Viscarin GP 109 NF) | Suspending agent/Viscosity | Ph. Eur. | | | 0.50 |
| Hydroxypropyl methylcellulose | modifier/Thickening agent | Ph. Eur. | | 0.50 | |
| (HPMC) | | | | | |
| Methyl cellulose | | Ph. Eur. | 0.60 | | |

| | | | |
|---|---|---|---|
| Total | 100.00 | 100.00 | 100.00 |

*Composition of the citrate-phosphate buffer: citric acid, sodium phosphate and water for injections (WFI).

The manufacturing process of diluent consists of a simple and conventional mixing of ingredients into the buffered solution and further filling in vials/bottles involving a number of well-established operations:
Step 1: Preparation of buffering agent (citrate-phosphate buffer pH 6.0)
Step 2: Preparation of the diluent by addition of excipients
Step 3: Filling into vials/bottles
Step 4: Quality control

Step 1: Preparation of citrate-phosphate buffer (pH 6.0). The scale and manufacturing vessels may be adjusted to match the final batch scale.

1. Weighing and dissolving
Preparation of 0.1M citric acid solution: required amount of citric acid is weighed and transferred to 3 L volumetric flask, then half of the water for injection required is added and solution is stirred. Once the solution is clear of particles, the remaining water is added to achieve 0.1M citric acid solution.
Preparation of 0.2M dibasic sodium phosphate solution—required amount of sodium phosphate salt (di basic sodium phosphate heptahydrate) is weighed and transferred to 5 L volumetric flask, then half of the water for injection required is added, and solution is stirred. Once the solution is clear of particulates, the remaining water is added to achieve 0.2M dibasic sodium phosphate solution.

### 2. Mixing

In a 15 L vessel, ~2196 mL of 0.1M citric acid solution and ~3804 mL 0.2M dibasic sodium phosphate solution is added and stirred to ensure a homogeneous mixture.

### 3. Final Buffer Solution

An initial pH is monitored; the required buffer solutions are added to achieve the target pH 6.0; then the vessel will be made to ca. 85% volume with water. The pH will be monitored and should the pH be outside of the target range (pH 6.00±0.05) the pH will be adjusted up or down using the sodium phosphate or citric acid solutions, respectively. The vessel will then be filled to achieve a total of 12 L with water and mixed for 30 sec before a final pH reading is taken.

### Step 2: Preparation of the Diluent:

### 4. Weighing and Dissolving

The required amount of buffer is weighed into a vessel for each diluent formulation and all remaining excipients (other than the polymer), are added in turn. Before the addition of the following excipient, a visual check is performed to ensure complete dissolution.
The solution is stirred until the resulting solution is clear and free of particulates.

### 5. Polymer Addition

Once all excipients are dissolved, the stirring speed is increased, and the polymer is added slowly and stirred until the diluent is clear and visually homogenous.
   Step 3: Filling into Vials/Bottles

### Step 4: Quality Control

### 4.5.8.5 Description and Composition of Drug Product

The drug product formulation containing ^{99m}Tc radiolabeled drug substance compound 1 is a white to off white, homogeneous suspension intended for nasal administration.

The nasal suspension will be manufactured extemporaneously by radiolabeling the compound 1 drug substance with ⁹⁹Tc tracer, suspending in diluent, and filled in 3 mL amber glass bottle assembled with Aptar V7 pump spray. Each bottle contains a 5 mm stainless steel bead to aid dispersion during manufacture and prior to administration of the nasal spray suspension.

One bottle will contain 2.5 g of radiolabeled product, which equates to 25 sprays per device at 4 MBq/100 μL, or 100 MBq/device in total. One dose (per nostril) contains ⁹⁹Tc compound 1 9 mg per 100 μL radiolabeled product.

For the phase 1 scintigraphy study, three diluent formulations have been developed, i.e., SU28, SU41 and SU45, and all three will be used for nasal suspension preparation. In addition, two different compound 1 particle sizes will be tested, referred to as small particle size (SPS) and large particle size (LPS), respectively.

The investigational medicinal products that will be evaluated in the planned scintigraphy clinical study along with their referenced name are listed in Table 40.

TABLE 40
Finished product abbreviations

| Investigational medicinal product | Reference in the IMPD |
|---|---|
| Radiolabeled compound 1, | ^{99m}Tc SU28SPS |
| 9 mg/nostril in 100 μL (18 mg) SU28₁₀ | |
| Radiolabeled compound 1, | ^{99m}Tc SU41SPS |
| 9 mg/nostril in 100 μL (18 mg) SU41₁₀ | |
| Radiolabeled compound 1, | ^{99m}Tc SU45SPS |
| 9 mg/nostril in 100 μL (18 mg) SU45₁₀ | |
| Radiolabeled compound 1, | ^{99m}Tc SU45LPS |
| 9 mg/nostril in 100 μL (18 mg) SU45₃₀ | |

The detailed compositions for all four investigational medicinal products are provided in Tables 41-44.

TABLE 41
Composition details of ^{99m}Tc SU28SPS

| Description | Ingredient | % w/w | Remark |
|---|---|---|---|
| API | Compound 1 (d90 = ~10 μm) | 9.00 | 9.00% |
| Diluent | Citrate-Phosphate Buffer pH 6.0 | 88.31 | 91.00% |
| (SU28) | Mannitol | 1.59 | |
| | Polyoxyl 40 Hydrogenate Castor | 0.46 | |
| | Oil (RH40) | | |
| | Potassium Sorbate | 0.09 | |
| | Methyl Cellulose | 0.55 | |
| | Total | 100.00 | 100.00 |
| Radioactive | ^{99m}Tc Sodium pertechnetate | | 100 MBq/ |
| Material | | | device in total |
| Packaging | Aptar VP7 Screw Top 3 ml | | 2.5 g product/ |
| | Pumps | | spray bottle |
| | Screw Top 3 ml Amber Bottles | | |

TABLE 42
Composition details of ^{99m}Tc SU41SPS

| Description | Ingredient | % w/w | Remark |
|---|---|---|---|
| API | Compound 1 (d90 = ~10 μm) | 9.00 | 9.00 |
| Diluent | Citrate-Phosphate Buffer pH 6.0 | 88.40 | 91.00 |
| (SU41) | Sorbitol | 1.59 | |
| | Polyoxyl 35 Castor Oil | 0.46 | |
| | Potassium Sorbate | 0.09 | |
| | HPMC | 0.46 | |
| | Total | 100.00 | 100.00 |
| Radioactive | ^{99m}Tc Sodium pertechnetate | | 100 MBq/ |
| Material | | | device in total |
| Packaging | Aptar VP7 Screw Top 3 ml | | 2.5 g product/ |
| | Pumps | | spray bottle |
| | Screw Top 3 ml Amber Bottles | | |

TABLE 43
Composition details of ^{99m}Tc SU45SPS

| Description | Ingredient | % w/w | Remark |
|---|---|---|---|
| API | Compound 1 (d90 = ~10 μm) | 9.00 | 9.00 |
| Diluent | Citrate-Phosphate Buffer pH 6.0 | 87.53 | 91.00 |
| (SU45) | EDTA | 0.18 | |
| | Sorbitol | 2.28 | |
| | Polyoxyl 35 Castor Oil | 0.46 | |
| | Potassium Sorbate | 0.09 | |
| | Carrageenan (Viscarin GP | 0.46 | |
| | 109 NF) | | |
| | Total | 100.00 | 100.00 |
| Radioactive | ^{99m}Tc Sodium pertechnetate | | 100 MBq/ |
| Material | | | device in total |
| Packaging | Aptar VP7 Screw Top 3 ml | | 2.5 g product/ |
| | Pumps | | spray bottle |
| | Screw Top 3 ml Amber Bottles | | |

TABLE 44
Composition details of ^{99m}Tc SU45LPS

| Description | Ingredient | % w/w | Remark |
|---|---|---|---|
| API | Compound 1 (d90 = ~30 μm) | 9.00 | 9.00 |
| Diluent | Citrate-Phosphate Buffer pH 6.0 | 87.53 | 91.00 |
| (SU45) | EDTA | 0.18 | |
| | Sorbitol | 2.28 | |
| | Polyoxyl 35 Castor Oil | 0.46 | |
| | Potassium Sorbate | 0.09 | |
| | Carrageenan (Viscarin GP | 0.46 | |
| | 109 NF) | | |
| | Total | 100.00 | 100.00 |
| Radioactive | ^{99m}Tc Sodium pertechnetate | | 100 MBq/ |
| Material | | | device in total |
| Packaging | Aptar VP7 Screw Top 3 ml | | 2.5 g product/ |
| | Pumps | | bottle |
| | Screw Top 3 ml Amber Bottles | | |

The ⁹⁹Technicium radiolabeled compound 1900 nasal spray, suspension was developed for limited clinical evaluation. It will be used only in the proposed phase 1 clinical pharmaco-scintigraphy study with the aim of demonstrating the ability to deliver compound 1 in a nasal spray form using a spray pump (Aptar VP7). The Aptar VP7 spray pump is a highly efficient pre-compression nasal spray pump which ensures consistent spray performance over a wide range of dose volumes (25 μl to 130 μl) and is suitable for liquid solutions, suspensions and viscous drug formulations. The Aptar VP7 spray pump is composed of: (1) spray pump (VP7AE/100; thread 18/415), (2) actuator (232 NA/B/R Actuator ASM); and (3) PP clip. Schematic drawings of the VP7 spray pump are provided in FIG. 22 and FIG. 23.

For investigational medicinal product 99Tech compound 1 9% Nasal spray, suspension the target dose volume is 100 μl.

Radiolabeling of the drug substance compound 1 via Technegas was selected as the preferred radiolabeling method, demonstrating a high binding affinity for the drug substance, suggesting that tracking of the compound 1 drug substance post patient administration, including location of the deposited spray particles within the nasopharynx will likely be obtained during a gamma scintigraphy.

Validation testing was performed to ensure radiolabeling of ^{99m}Tc SU28_{SPS}, ^{99m}Tc SU41_{SPS}, ^{99m}Tc SU45_{SPS} and ^{99m}Tc SU45_{LPS} did not have any significant effect on the performance.

Due to the short half-life of the radioisotope, ^{99m}Tc (~6 h), the formulations ^{99m}Tc SU28_{SPS}, ^{99m}Tc SU41_{SPS}, ^{99m}Tc SU45_{SPS} and ^{99m}Tc SU45_{LPS} will be manufactured within 12 hours prior to administration.

Radiolabeling Procedure for compound 1 (API): The radiolabeling process involves the physical association of Technegas particles with the API dry powder. Sodium pertechnetate (Na⁺TcO₄⁻) will be utilized as the required radionuclide for use in the Technegas generator. It is received in a liquid form and is processed within the Technegas generator to produce carbon nanoparticles labelled with ^{99m}Tc. These particles comprise hexagonal flat crystals of ^{99m}Tc encased in multiple layers of carbon such that the ^{99m}Tc metal is protected from the environment, thus preventing it from oxidizing and forming pertechnetate. Radiolabel ^{99m}Tc sodium pertechnetate will be loaded into a carbon crucible and placed into the Technegas Generator, where radioactive aerosolized particles are created at high temperature. These particles are drawn through a chamber containing the API dry powder using a vacuum pump. 6% Ethylenediaminetetraacetic acid (EDTA) solution is employed as a trap for non-absorbed Technegas particles, by chelation.

Three aliquots of API are radiolabeled to manufacture up to four devices required per study day. All radiolabeled API aliquots (3 in total) are combined by manually mixing using rotation for 5 minutes within a sealed bijou behind lead shielding. The total radioactive dose is recorded using Capintec dose calibrator.

The API (radiolabeled) and diluent are combined within the nasal spray pump/device, along with a 5 mm stainless steel bead to aid dispersion of the insoluble API. Mixing of the product will be performed for 20 minutes using a Turbula mixer at speed setting 3.

The actual and predicted (at scheduled dosing time) radiolabeled dose of each radiolabeled nasal spray pump/device is determined during manufacture using a dose calibrator. Individual radiolabeled product (2500 mg in spray pump) will be prepared per trial participant for use in the study under the direct supervision of authorized personnel and are subject to final QP release.

Since during the clinical study, radiolabeled samples are manufactured and administered within a 12-hour timeframe, an extended QC testing cannot be performed on products manufactured for dosing, therefore the validation work was undertaken to provide confidence that the proposed method of radiolabeling will allow the clinical study objectives to be met and that samples can be manufactured reproducibly.

Process Validation. For the validation study, four small-scale formulations of radiolabeled drug products were prepared manually. At the same time, four small-scale non-radiolabeled formulations of the drug product, using the same manufacturing process described above but excluding the radiolabeling process part, were reconstituted manually at the same site as the radiolabeled formulations.

To confirm the radioactive material level as appropriate marker to trace the location of material in nasal cavity using gamma scintigraphy over period of maximum 12 hours, the radiolabeled drug product, the process parameters that were deemed critical to the radiolabeling manufacturing process, and pharmaceutical quality and were documented during the validation as summarized in Table 45.

TABLE 45
Testing parameters for radiolabeled product

| | | |
|---|---|---|
| Quality Attribute | Acceptance Criteria | Test Method |

Radiolabeled and Non-Radiolabeled Suspension 9 mg/100 μl. SU28, SU41 and SU45
(with both small and large PSD DS) in 3 ml Amber Aptar VP7 Pump Spray Device

| Appearance/ | White to off white, visually homogeneous, | | Visual inspection |
|---|---|---|---|
| Redispersibility | low viscosity suspension. Sediment of | | |
| | clear layer of liquid may be present which | | |
| | is readily redispersed on shaking | | |
| Appearance/package | Amber glass 3 ml bottle equipped with | | Visual inspection vs |
| description | Aptar Vp7 spray pump and cap | | reference primary package |
| Mean delivered dose after | 75-125% weight range (mg) based on | | HPLC |
| priming | label claim | | |
| Mean spray weight after | Within 75-125% Weight Range (mg) | | Weight check |
| priming | based on label claim | | |
| Total Radiolabel uptake | 30-200 | MBq | Capintec ™ Dosimeter |
| by API (350 mg) | | | |
| Radiolabel Dose Per | 10-100 | MBq | Capintec ™ Dosimeter |
| Product at TOD | | | |
| Radiolabel Dose Per | 0.8-4 | MBq | Capintec ™ Dosimeter |

| | | |
|---|---|---|
| Spray at TOD | | |
| Number of Priming | 4 sprays | Weight check |
| Sprays Based on Spray | | |
| Weight | | |

In addition, to demonstrate that the radiolabeling procedure does not alter the product performance and pharmaceutical quality, the tests performed during the validation study on both non-radiolabeled and “cooled off” formulations are summarized in Table 46.

TABLE 46
Testing parameters for “cooled off” radiolabeled and non-radiolabeled product

| Test | Analytical Method | Acceptance criteria |
|---|---|---|
| Compound 1 Identification | HPLC | The retention time of compound 1 peak |
| | | obtained from the sample preparation is |
| | | within ±2% of the retention time of |
| | | compound 1 peak obtained from |
| | | the reference preparation |
| Compound 1Assay (content | HPLC | 90.0-110.0% of label claim |
| uniformity) | | |
| Compound 1Related | HPLC | Report RRT and % w/w for all related |
| substances | | substances of 0.05% or greater |
| | | Any unspecified degradation products NMT |
| | | 0.30% w/w |
| | | Total impurities NMT 3.0% w/w |
| Macroscopic appearance | Visual | White to off white, visually homogeneous, |
| | | low viscosity suspension |
| | | Sediment or clear layer of liquid may be |
| | | present which is readily redispersed on |
| | | shaking, to give a |
| | | visually homogeneous suspension |
| Microscopic observations | Microscopy | Report result |
| Apparent pH | pH meter | From 5.5 to 6.5 |
| Osmolarity | Osmometer | Report result |
| Particle size | Laser diffraction | Report result |
| Droplet size | Laser diffraction | Report result |
| Delivered dose uniformity* | DUSA | 75.0-125.0% of label claim |
| Mean spray weight after | Weight check | 75.0-125.0% range, based on density and |
| priming* | | 9% label claim |
| Microbial quality testing* | | |
| Total aerobic microbial count | Ph. Eur. 2.6.12 | ≤10² CFU/g or CFU/mL |
| (TAMC), CFU/g | USP<61> | |
| Total yeasts and moulds | Ph. Eur. 2.6.13 | ≤10¹ CFU/g or CFU/mL |
| count (TYMC), CFU/g | USP<61> | |
| Staphylococcus aureus | Ph. Eur. 2.6.13 | Absent in 1 g or 1 mL |
| | USP<62> | |
| Pseudomonas aeruginosa | Ph. Eur. 2.6.13 | Absent in 1 g or 1 mL |
| | USP<62> | |

*Tests performed only on the non-radiolabeled formulations.

Radioactive uptake by compound 1 drug substance. The results of total radiolabeled uptake by compound 1 drug substance aliquot of 350 mg are shown in Table 47.

TABLE 47
Total radiolabeled dose (MBq) uptake by API

| | Total Radiolabel Dose (MBq) |
|---|---|
| Product Description | Uptake by drug substance |
| ^{99m}Tc SU28_{SPS} | 42.5-123 |
| ^{99m}Tc SU41_{SPS} | 75.5-91.5 |
| ^{99m}Tc SU45_{SPS} | 37.1-58.1 |
| ^{99m}Tc SU45_{LPS} | 150.7-190.6 |

The results demonstrate that total radiolabel dose uptake by drug substance is found in range of 37.1-190.6 MBq per compound 1 drug substance aliquot (350 mg).

Radiolabel Dose Uniformity Evaluation. The results of the radiolabel uniformity in sprayed product at time of delivery (TOD) (mean delivered dose after priming, per 100 μl) is tabulated in Table 48.

TABLE 48
Mean radiolabeled dose (MBq) for dispensed
product at TOD (N = 10 Average)

| Product | Mean Radiolabel Dose | |
|---|---|---|
| Description | (MBq) at TOD | SD |
| ^{99m}Tc SU28_{SPS} | 1.510 | 0.067 |
| ^{99m}Tc SU41_{SPS} | 1.262 | 0.034 |
| ^{99m}Tc SU45_{SPS} | 0.861 | 0.185 |
| ^{99m}Tc SU45_{LPS} | 3.099 | 0.070 |

The results demonstrate that mean radiation doses of around 0.9-3.1 MBq were observed. Difference in mean radiolabel doses observed could be because of different drug substance PSD or diluent with varying density. A specification of 0.8-4 MBq per 100 μl spray at TOD was therefore recommended.

The mean radiolabel dose in the devices (2.5 g product in 3 mL spray bottle) at TOD is tabulated in Table 49.

TABLE 49
Mean Radiolabel Total Dose Per Device at TOD (N = 4 Average)

| Product | Mean Radiolabel Dose | |
|---|---|---|
| Description | (MBq) at TOD | SD |
| ^{99m}Tc SU28_{SPS} | 31.70 | 1.66 |
| ^{99m}Tc SU41_{SPS} | 27.25 | 1.36 |
| ^{99m}Tc SU45_{SPS} | 18.32 | 1.68 |
| ^{99m}Tc SU45_{LPS} | 65.3 | 4.59 |

Total radiation dose per device at TOD is observed in the range of 18 to 65 MBq. Therefore, the specification is set to be 10-100 MBq per device at TOD.

Priming Sprays. The number of priming sprays was confirmed by recording the dispensed weight for each actuation and establishing when the values were within the expected range. Priming was required to remove the air from the pump and actuator tubing, replacing with formulation ready for dosing. Initial actuations are not intended to be dosed as they dispense low product weights, which are outside the 75-125% nominal spray weight containing 9 mg compound 1 drug substance (product density dependent). The number of priming sprays for the products, and the associated dispensed weights are shown in Table 50.

TABLE 50
Priming spray checks

| | Radiolabeled Product | | | | | | | |
|---|---|---|---|---|---|---|---|---|
| | ^{99m}Tc | ^{99m}Tc | ^{99m}Tc | ^{99m}Tc | Non-radiolabeled product | | | |
| Product | SU28SPS | SU41SPS | SU45SPS | SU45LPS | SU28SPS | SU41SPS | SU45SPS | SU45LPS |
| Initial | 0.00 | 0.00 | 0.00 | 0.00 | 0.00 | 0.00 | 0.00 | 0.00 |
| Spray 1 | 4.00 | 4.00 | 0.00 | 0.24 | 0.00 | 0.00 | 0.83 | 0.35 |
| Spray 2 | 19.94 | 19.94 | 0.16 | −0.08 | 14.00 | 0.00 | 0.29 | −0.10 |
| Spray 3 | 95.40 | 95.40 | 70.04 | 67.3 | 84.90 | 17.70 | 55.84 | 69.16 |
| Spray 4 | — | — | 113.79 | 112.19 | — | 97.40 | 114.34 | 113.45 |
| No. of Priming | 3 | 3 | 4 | 4 | 3 | 4 | 4 | 4 |
| Sprays | | | | | | | | |

The number of required priming weights was therefore determined to be ≥4.

Mean Delivered Dose Weight Evaluation. Radiolabel and non-radiolabel product dose checks done after priming sprays and data are presented in Table 51.

TABLE 51
Mean delivered dose weight after priming sprays (n = 10)

| | | | |
|---|---|---|---|
| | | Mean Dispensed | |
| Product Description | Radiolabeled | Weight (mg) | SD |

Radiolabeled Product

| | | | |
|---|---|---|---|
| ^{99m}Tc SU28_{SPS} | Y | 113.64 | 4.93 |
| ^{99m}Tc SU41_{SPS} | Y | 114.82 | 2.52 |
| ^{99m}Tc SU45_{SPS} | Y | 99.69 | 22.40 |
| ^{99m}Tc SU45_{LPS} | Y | 120.37 | 2.83 |

Non-radiolabeled Product

| | | | |
|---|---|---|---|
| SU28_{SPS} | N | 105.15 | 7.87 |
| SU41_{SPS} | N | 113.74 | 4.66 |
| SU45_{SPS} | N | 113.72 | 4.71 |
| SU45_{LPS} | N | 115.97 | 7.52 |

Mean dispensed/delivered weight was observed to be lower and more variable for radiolabeled ^{99m}Tc SU45_{SPS} compared to ^{99m}Tc SU28_{SPS}, ^{99m}Tc SU41_{SPS} and ^{99m}Tc SU45_{LPS}; however, results fell within expected range. This equates to 96.50% of the expected mean dispensed weight (103.30 mg) in SU45 diluent. Non-radiolabeled SU45_{SPS} data was within trend.

Mean Delivered Dose After Priming (Content Uniformity) Evaluation. Delivered dose is the amount of drug emitted from the drug device that is available to the user, when the device is actuated correctly. Delivered Dose Uniformity (DDU) was measured by firing the test device into a dosage unit sampling apparatus (DUSA) which contained a filter. The active drug caught on the filter was then dissolved in solvent and analyzed using the same HPLC analytical method as used for compound 1 identification. The mean delivered doses (after priming) for radiolabeled and non-radiolabeled products are shown in Table 52, for n=10.

TABLE 52
Mean delivered dose using content uniformity

| | | | | |
|---|---|---|---|---|
| | | | Label | Predicted Dosed |
| | Radio- | CU | Claim/ | Amount of drug |
| Product Description | labeled | Value (%) | 100 μl | substance (mg)* |

Radiolabeled Product (after decayed)

| | | | | |
|---|---|---|---|---|
| ^{99m}Tc SU28_{SPS} | Y | 103.43 | 9.31 | 9.91 |
| ^{99m}Tc SU41_{SPS} | Y | 105.78 | 9.52 | 10.00 |
| ^{99m}Tc SU45_{SPS} | Y | 107.47 | 9.67 | 8.69 |
| ^{99m}Tc SU45_{LPS} | Y | 108.28 | 9.75 | 10.49 |

Non-radiolabeled Product

| | | | | |
|---|---|---|---|---|
| SU28_{SPS} | N | 107.89 | 9.71 | 9.17 |
| SU41_{SPS} | N | 107.81 | 9.70 | 9.91 |
| SU45_{SPS} | N | 108.19 | 9.74 | 9.91 |
| SU45_{LPS} | N | 108.81 | 9.79 | 10.10 |

Mean delivered dose after priming was observed to be slightly lower for radiolabeled ^{99m}Tc SU45_{SPS} compared to ^{99m}Tc SU28_{SPS}, ^{99m}Tc SU41_{SPS} and ^{99m}Tc SU45_{LPS}; however, results fell within expected range. This equated to 96.33% of the theoretical dose of 9 mg/100 μL.

In order to demonstrate that the radiolabelling procedure did not alter the product quality and performance, a comparison between the properties/characteristics of non-radiolabeled product vs “cooled off” product was performed and summarized in Table 53.

TABLE 53
Comparison between the properties/characteristics of non-radiolabeled product vs “cooled off” product

| | | | | | | “Cooled-off” formulations* | | | |
|---|---|---|---|---|---|---|---|---|---|
| | | Non-radiolabeled material* | | | | ^{99m}Tc | ^{99m}Tc | ^{99m}Tc | ^{99m}Tc |
| Test | Acceptance criteria | SU28_{SPS} | SU41_{SPS} | SU45_{SPS} | SU45_{LPS} | SU28_{SPS} | SU41_{SPS} | SU45_{SPS} | SU45_{LPS} |
| Compound 1 | The retention time of | Conforms | Conforms | Conforms | Conforms | Conforms | Conforms | Conforms | Conforms |
| Identification | Compound 1 | | | | | | | | |
| | peak obtained from the | | | | | | | | |
| | sample preparation is | | | | | | | | |
| | within ±2% of the | | | | | | | | |
| | retention time of | | | | | | | | |
| | Compound 1 | | | | | | | | |
| | peak obtained from the | | | | | | | | |
| | reference preparation | | | | | | | | |
| Compound 1 | 90.0-110.0% of label | 99.9 | 99.7 | 100.3 | 92.9 | 97.0 | 99.7 | 101.5 | 98.4 |
| Assay (content | claim | | | | | | | | |
| uniformity) | | | | | | | | | |
| Compound 1 | Report RRT and % w/w | RRT = | RRT = | RRT = | RRT= | RRT = | RRT = | RRT = | RRT= |
| Related | for all related substances | 1.05: 0.11% | 1.05: 0.11% | 1.05: 0.11% | 1.05: 0.10% | 1.05: 0.11% | 1.05: 0.11% | 1.05: 0.11% | 1.05: 0.09% |
| substances | of 0.05% or greater | | | | RRT = | | | | |
| | Any unspecified | | | | 1.43: 0.16% | | | | |
| | degradation products | | | | | | | | |
| | NMT 0.30% w/w | | | | | | | | |
| | Total impurities NMT | 0.11% | 0.11% | 0.11% | 0.26% | 0.11% | 0.11% | 0.11% | 0.09% |
| | 3.0% w/w | | | | | | | | |
| Macroscopic | White to off white, | Conforms | Conforms | Conforms | Conforms | Conforms | Conforms | Conforms | Conforms |
| appearance | visually homogeneous, | | | | | | | | |
| | low viscosity suspension | | | | | | | | |
| | Sediment or clear layer | | | | | | | | |
| | of liquid may be present | | | | | | | | |
| | which is readily | | | | | | | | |
| | redispersed on shaking, | | | | | | | | |
| | to give a visually | | | | | | | | |
| | homogeneous suspension | | | | | | | | |
| Apparent pH | From 5.5 to 6.5 | 6.1 | 6.4 | 6.1 | 6.0 | 6.2 | 6.2 | 6.1 | 6.1 |
| Delivered dose | 75.0-125.0% of label | 95.7-100.5 | 98.9-101.3 | 95.4-100.2 | 80.5-101.7 | | | | |
| uniformity | claim | | | | | | | | |
| Mean spray | 75.0-125.0% range, | 110.7 | 109.0 | 111.7 | 115.9** | | | | |
| weight after | based on density and 9% | | | | | | | | |
| priming | label claim | | | | | | | | |

Microbial quality testing

| | | | | | |
|---|---|---|---|---|---|
| Total aerobic | ≤10² CFU/g or CFU/mL | ≤10¹ CFU/mL | ≤10¹ CFU/mL | ≤10¹ CFU/mL | ≤10¹ CFU/mL |
| microbial count | | | | | |
| (TAMC), CFU/g | | | | | |
| Total yeasts and | ≤10¹ CFU/g or CFU/mL | ≤10¹ CFU/mL | ≤10¹ CFU/mL | ≤10¹ CFU/mL | ≤10¹ CFU/mL |
| moulds count | | | | | |
| (TYMC), CFU/g | | | | | |
| Staphylococcus | Absent in 1 g or 1 mL | Not isolated in 1 mL | Not isolated in 1 mL | Not isolated in 1 mL | Not isolated in 1 mL |
| aureus | | | | | |
| Pseudomonas | Absent in 1 g or 1 mL | Not isolated in 1 mL | Not isolated in 1 mL | Not isolated in 1 mL | Not isolated in 1 mL |
| aeruginosa | | | | | |

Characterization tests

| | | | | | | | | | |
|---|---|---|---|---|---|---|---|---|---|
| Microscopic | Report result | Visible drug | Visible drug | Visible drug | Visible drug | Visible drug | Visible drug | Visible drug | Visible drug |
| observations | | particulates | particulates | particulates | particulates | particulates | particulates | particulates | particulates |
| Osmolarity | Report result | 289 mOsm/kg | 288 mOsm/kg | 338 mOsm/kg | 344 mOsm/kg | 293 mOsm/kg | 296 mOsm/kg | 349 mOsm/kg | 352 mOsm/kg |
| Particle size | Report result | D90 - 10.02 μm | D90 - 17.70 μm | D90 - 12.17 μm | D90 - 33.03 μm | D90 - 7.28 μm | D90 - 15.07 μm | D90 - 8.70 μm | D90 - 32.72 μm |
| Droplet size | Report result | 89.17 ± | 953.05 ± | 726.06 ± | 824.26 ± | 126.36 ± | 763.28 ± | 602.27 ± | 689.25 ± |
| | | 4.53 μm | 200.85 μm | 254.40 μm | 201.26 μm | 55.98 μm | 201.12 μm | 142.41 μm | 161.06 μm |

### 4.5.8.1 Study Protocol

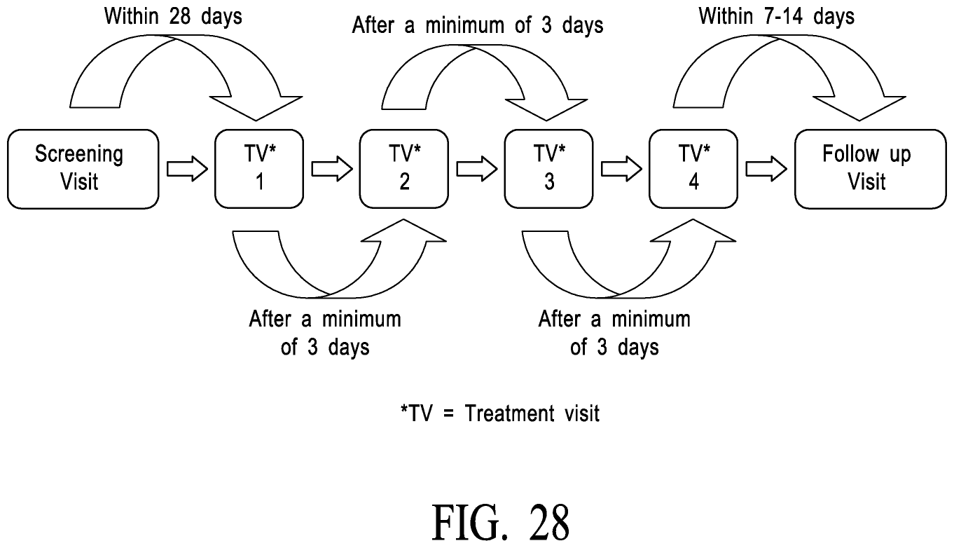

This is a single center, open label, four arm cross-over study in 8 healthy male and female volunteers to assess a single dose of four formulations of nasally delivered compound 1. Participants will visit the site on 6 times including the screening and follow up visit. The first treatment administration will occur within 28 days of screening. Each dosing occasion will be separated by a minimum of 3-day washout period. A follow-up visit will take place no earlier than 7 days and no later than 14 days after the participant's last Treatment Visit. The trial design is depicted in FIG. 28. Each treatment will be radiolabeled with Technetium-99 (^{99m}Tc) and a gamma camera will be used to track behavior post administration. The technique of gamma scintigraphy is a valuable tool in evaluating the in vivo performance of pharmaceutical dosage forms. Scintigraphy is a non-invasive procedure which provides information on deposition and movement of the formulation. The radioactive load is minimal, and all procedures are well established. Scintigraphic analysis will be used to visualize the deposition of the radiolabeled product within the nasal cavity and monitor retention time. This study combines nasal scintigraphic imaging with blood sampling for PK analysis to assess the behavior of the novel nasal formulations of compound 1.

### 4.5.8.2 Objectives and Endpoints

Primary Objective: To evaluate the safety and tolerability of 4 different formulations of a single dose of 18 mg compound 1 via nasal administration in healthy male and female adult participants.

### Primary Endpoints:

Incidence, type, timing, severity and relatedness of treatment emergent adverse events (including SAEs)
Safety bloods at baseline, 24 hours post each dose and follow up visit
Urinalysis at baseline and at follow up visit
12 lead ECG (HR, QRS and QTc intervals) at baseline, pre-determined timepoints post dose and at follow up visit
Vital signs at baseline, pre-determined timepoints post dose and at follow up visit
Clinically significant change in physical examination as assessed at screening, treatment visit and follow up visit.

### Secondary Objectives:

Assess behavior via scintigraphy of 4 different formulations of compound 1 after a single dose of 18 mg via nasal administration.
Determine the plasma pharmacokinetics of 4 different formulations of compound 1 after a single dose of 18 mg via nasal administration.

### Secondary Endpoints:

Location of radiolabel within nasal cavity with time
Relative amount of radiolabel within nasal cavity with time (with amount at time 0 being expressed as 100%).
Total duration of radiolabel retention within nasal cavity
Samples taken for bioanalysis at stated timepoints for the following PK parameters: Maximum Concentration (Cₘₐₓ), Time of Maximum Concentration (Tₘₐₓ), Lag Time (T_{lag}), Area Under the Curve from time 0 to 24 hours (AUCₗₐₛₜ), Area Under the Curve from time 0 to infinity (AUC_{0-inf}), Terminal First Order Rate constant (K), and Terminal Elimination Half Life (T_{1/2}).

### 4.5.8.3 Study Population

A total of 8 healthy male and female volunteers will be recruited in this proof-of-concept study with the aim of having at least 6 evaluable participants (an evaluable participant being one who receives all 4 doses). It is essential to ensure they do not suffer from any significant acute or chronic nasal disorders, which could impact on the validity of the scintigraphy deposition and retention data. This will be assessed at each treatment visit. Any participants with a BMI above 32 kg/m² will be excluded as shielding caused by bone, muscle, other organs and soft tissue will attenuate gamma rays.

Inclusion Criteria. Participants are eligible to be included in the study only if all of the following criteria apply:
1. Age—Aged between 18 and 65 years inclusive.
2. Weight & Body mass index (BMI)—BMI between 18 and 32 kg/m², inclusive.
3. Compliance—Understands and is willing, able, and likely to comply with all study procedures and restrictions.
4. Consent—Demonstrates understanding of the study and willingness to participate as evidenced by voluntary written informed consent (signed and dated) obtained before any trial-related activities.
5. General health—Healthy (as determined by the Investigator) based upon the results of a medical history, physical examination (including nasal exam), vital signs, 12 lead ECG and clinical laboratory safety tests.
6. Tolerance of Nasal Spray—Demonstrates ability to tolerate administration of nasal spray to each nostril at screening visit as assessed by investigator. Investigator or appropriately skilled designee can administer up to a maximum of three doses of 0.9% saline (one dose=100 μL in each nostril). Ability to tolerate this will be judged and documented by the investigator. A minimum tolerance of one spray in each nostril should be achieved.
7. Family Planning/Contraception
   i. Male Participants:

Male participants are eligible to participate if they agree to the following during the study intervention period and for at least 90 days after the last dose of study intervention:
Refrain from donating sperm PLUS, either:
   Be abstinent from heterosexual intercourse as their preferred and usual lifestyle (abstinent on a long term and persistent basis) and agree to remain abstinent or
   Must agree to use a male condom alongside female partner use of an additional highly effective contraceptive method with a failure rate of <1% per year when having sexual intercourse with a woman of childbearing potential who is not currently pregnant.
   ii. Female Participants:

A female participant is eligible to participate if she is not pregnant or breastfeeding, and one of the following conditions applies:
Is a woman of nonchildbearing potential (WONCBP) or
Is a WOCBP and using a contraceptive method that is highly effective (with a failure rate of <1% per year), with low user dependency during the study intervention period and at least until the follow up visit of the study. The investigator should evaluate the potential for contraceptive method failure (e.g., noncompliance, recently initiated) in relationship to the first dose of study intervention.

Exclusion Criteria. Participants meeting any of the following criteria during eligibility assessments will be excluded from trial participation:
a. Medical History
   i. Current or recurrent disease/condition that, in the opinion of the investigator could affect study conduct; the safety of the participant as a result of participation; and/or the ability of the participant to complete the study or laboratory assessments (including but not exclusively a history of hay fever, rhinitis, asthma, syncope or hypertension).
   ii. Current or relevant previous history of severe or unstable psychiatric illness, that may require treatment or make the participant unlikely to fully complete the study, or that presents undue risk from the study medication or procedures.
   iii. Having any illness (e.g., active allergy, fever, hypersensitivity reaction) judged by the investigator as clinically significant in the 3 months prior to first dosing.
   iv. Haematological or biochemical blood test at screening outside normal ranges and deemed clinically significant by the PI or medically qualified designee.
   v. Clinically significant physical examination or clinically significant investigations (including those performed at screening and during treatment arm), deemed by the investigator to render the volunteer unfit for the study.
   vi. Measured body temperature>38° C. (at screening or dosing visit)
   vii. Rhinitis (on or off therapy) and/or stuffy nose in the 2 weeks prior to first dosing.
   viii. Positive serology suggestive of acute or chronic infection with HIV, Hep. B (HBsAg positive) or HCV.
   ix. History of hepatitis from any cause, with the exception of hepatitis A, that was resolved within 3 months prior to first dose.
   x. Positive COVID-19 lateral flow test at screening or at treatment visit.
   xi. Prolonged corrected QT interval (as calculated by Fridericia and Framingham formulae) on 12 lead ECG as defined by QTc>460 (females) and 450 (male) msec by either calculation
b. Medications
   i. Use of nasal sprays, rinses or douches in the 2 weeks prior to first and/or any subsequent dose. Other than saline nasal spray administered at screening to test tolerance and any nasal spray dosages administered as per protocol.
   ii. Participant is scheduled to take prescribed medication within 14 days (or 5 half lives—whichever is longer) prior to the first dose of compound 1 which, in the opinion of the investigator, will interfere with the study procedures or compromise safety. Permitted prescribed medications are oral/IM/implantable contraceptives.
   iii. Participant is scheduled to take over-the-counter (OTC) medication, including vitamins, pro and prebiotics and natural or herbal remedies, within 48 hours prior to the first dose unless approved by the investigator.
c. Alcohol/Substance Abuse
   i. Recent history (within the last year) of alcohol or other substance abuse.
   ii. Previous regular/habitual use of inhaled recreational drugs
   iii. Participant has an average weekly alcohol intake of greater than 14 units.
   iv. Participant has positive urine drugs of abuse test at screening or prior to dosing evaluation.
   v. Participant has a positive breath alcohol test at screening or prior to dosing evaluation. (breath testing may be repeated once at investigator discretion within a 5 minute window of first test)
d. Smoking
   i. Participant has recently discontinued smoking or vaping (less than 3 months)
   ii. Participant is currently a smoker, vaper or user of nicotine-containing products.
   iii. Participant has a positive urine cotinine test at screening or prior to dosing evaluation.

### 4.5.8.4 Study Interventions

Participants will be administered the following treatments (Table 54):
a. Treatment 1: 2 administrations of 9 mg compound 1 in 100 μL (one spray in each nostril=total dose of 18 mg) SU28₁₀
b. Treatment 1: 2 administrations of 9 mg compound 1 in 100 μL (one spray in each nostril=total dose of 18 mg) SU45₁₀
c. Treatment 1: 2 administrations of 9 mg compound 1 in 100 μL (one spray in each nostril=total dose of 18 mg) SU41₁₀
d. Treatment 1: 2 administrations of 9 mg compound 1 in 100 μL (one spray in each nostril=total dose of 18 mg) SU45₁₀

No reference product is planned in this study. The product will be radiolabeled to contain a maximum of 2 MBq ^{99m}Tc per spray (maximum total 4MBq) at time of dosing. The radiolabeled nasal sprays will be administered by an authorized member of the study site personnel and the administration witnessed by a second member of clinical staff. The PI or medically qualified designee will be onsite for dosing and for 4 hours post dosing. The PI or medical designee will be on-call for the remainder of each treatment visit.

Participants will be dosed in an upright position then must lie down immediately post dosing. Participants are to remain in a supine position or can have back elevated to a 45-degree angle to the horizontal for 18 hours post dose. Exceptions are permitted for toilet breaks and food intake.

TABLE 54
Study Interventions To Be Administered

| | Intervention Name | | | |
|---|---|---|---|---|
| | Treatment 1 | Treatment 2 | Treatment 3 | Treatment 4 |
| Formulation | SU28 | SU45 | SU41 | SU45 |
| (diluent) | | | | |
| Compound 1 | <10 μM | <10 μM | <10 μM | <35 μM |
| particle size | | | | |
| (D90) | | | | |
| Intervention | Single dose 18 | Single dose 18 | Single dose 18 | Single dose 18 |
| Description | mg via nasal | mg via nasal | mg via nasal | mg via nasal |
| | spray | spray | spray | spray |
| Type | Nasal Micro | Nasal Micro | Nasal Micro | Nasal Micro |
| | Suspension | Suspension | Suspension | Suspension |
| | in spray | in spray | in spray | in spray |
| | pump | pump | pump | pump |
| Dose | Liquid for nasal | Liquid for nasal | Liquid for nasal | Liquid for nasal |
| Formulation | administration | administration | administration | administration |
| Unit Dose | 9 mg per 100 μL | 9 mg per 100 μL | 9 mg per 100 μL | 9 mg per 100 μL |
| Strength(s) | | | | |
| Dosage Level(s) | 1 spray of 100 | 1 spray of 100 | 1 spray of 100 | 1 spray of 100 |
| | μL in each | μL in each | μL in each | μL in each |
| | nostril, single | nostril, single | nostril, single | nostril, single |
| | dose | dose | dose | dose |
| Route of | Nasal | Nasal | Nasal | Nasal |
| Administration | | | | |

Study procedures, their timing and their allowed timing windows are summarized in the Schedule of Activities (SOA; Table 55). Adherence to the study design requirements, including those specified in the SoA, is essential and required for study conduct.

TABLE 55
Schedule of Activities

| | | | | | | | | | | | | | | | | | | | | | | Notes |
|---|---|---|---|---|---|---|---|---|---|---|---|---|---|---|---|---|---|---|---|---|---|---|
| | | | | | | | | | | | | | | | | | | | | | | E/D = Early |
| | Screening | | Treatment Visits 1, 2, 3, 4¹ | | | | | | | | | | | | | | | | | | Follow-up or E/D Visit | Discontinuation |
| | Days | | | | | | | | | | | | | | | | | | | | | |
| | | | | | | | | | | | | | | | | | | | | | Within 7- 14 days | |
| | Up to −28 | 1 | | | | | | | | | | | | | | | | | | 2 | of final dose | |
| | Time post dose (minutes [′]/h) Dose assumed to be around or before 2pm | | | | | | | | | | | | | | | | | | | | | |
| Procedure | — | −2 | −1 | 0 | 10′ | 15′ | 30′ | 45′ | 1 | 2 | 3 | 4 | 5 | 6 | 7 | 8 | 9 | 10 | 18 | 24 | — | — |

| | | | | |
|---|---|---|---|---|
| Informed consent | X | | | |
| Admission to Unit/Enrolment | | X | | |
| Discharge from Unit | | | X | |
| Ambulatory Visit | X | | | X |

Participant evaluation

| | | | | | | | | | | | | | |
|---|---|---|---|---|---|---|---|---|---|---|---|---|---|
| Inclusion/Exclusion | X | | | | | | | | | | | | |
| Eligibility Assessment | X | X | | | | | | | | | | | |
| Demography | X | | | | | | | | | | | | |
| Height and Weight | X | | | | | | | | | | | | |
| Full physical examination | X | | | | | | | | | | | X | |
| (including nasal examination | | | | | | | | | | | | | |
| at screening) | | | | | | | | | | | | | |
| Brief physical examination | | | | | | X | | | | | | | See Section 8.2.1 |
| including nasal examination | | | | | | | | | | | | | |
| Relevant Medical history | X | | | | | | | | | | | | Substances: [drugs, alcohol, |
| (includes substance use) | | | | | | | | | | | | | nicotine] |
| Highly sensitive serum OR | X | X | | | | | | | | | | X | [refer to Section 8.2.5 |
| urine pregnancy test | | | | | | | | | | | | | Pregnancy Testing for |
| (WOCBP only) | | | | | | | | | | | | | instruction on timepoints] |
| Urine drug, cotinine, and breath | X | X | | | | | | | | | | | |
| alcohol screen tests | | | | | | | | | | | | | |
| COVID-19 (lateral flow) test | X | X | | | | | | | | | | | |
| HIV, Hepatitis B and C screening | X | | | | | | | | | | | | |
| Laboratory tests for safety bloods | X | | | | | | | | | | X | X | 24 hour safety bloods to be |
| | | | | | | | | | | | | | taken at same time as 24 hour |
| | | | | | | | | | | | | | PK sample. |
| Urinalysis | X | | | | | | | | | | | X | |
| 12-lead ECG | X | | | X | | | X | | X | X | | X | +/−15 min |
| Vital signs | X | X | X | X | X | X | X | X | X | X | X | X | +/−15 min |
| Concomitant Medication History | X | X | | | | | | | | | | X | |

Study Specific Procedures

| Study intervention/administration | | X | | | | | | | | | | | | | | | | |
|---|---|---|---|---|---|---|---|---|---|---|---|---|---|---|---|---|---|---|
| of study medication | | | | | | | | | | | | | | | | | | |
| Scintigraphic Imaging | | ←---→ | X | X | X | X | X | X | X | X | X | X | X | X | X | | | Dynamic scintigraphic |
| | | | | | | | | | | | | | | | | | | imaging will be performed from |
| | | | | | | | | | | | | | | | | | | 0-10 minutes followed by |
| | | | | | | | | | | | | | | | | | | static imaging at 15, 30 and 45 |
| | | | | | | | | | | | | | | | | | | min post dose then hourly |
| | | | | | | | | | | | | | | | | | | from 1 to maximum of 10 |
| | | | | | | | | | | | | | | | | | | hours post dose. Permissible |
| | | | | | | | | | | | | | | | | | | window +/−2 min of |
| | | | | | | | | | | | | | | | | | | scheduled time for images |
| | | | | | | | | | | | | | | | | | | taken up to 60 mins post |
| | | | | | | | | | | | | | | | | | | dose. +/−5 min for imaging |
| | | | | | | | | | | | | | | | | | | between 1 and 10 hours. |
| PK blood sampling | X | | X | X | X | X | X | X | X | X | X | X | X | X | X | X | X | Sample to be taken within 1 |
| | | | | | | | | | | | | | | | | | | hour pre-dose, then at stated |
| | | | | | | | | | | | | | | | | | | time points with +/−5 min |
| | | | | | | | | | | | | | | | | | | permissible window. |

| | | | |
|---|---|---|---|
| AE Assessment | X | ←-------------------------------------------------------------------------------------------------------------- | X |
| | | -------------→ | |

### 4.5.8.5 Study Assessments

Screening Visits. All screening evaluations must be completed and reviewed to confirm that potential participants meet all eligibility criteria. The PI will maintain a screening log to record details of all participants screened and to confirm eligibility or record reasons for screening failure, as applicable. If a lab test result is outside of normal range and is deemed CS, or if there was a technical issue with the sample the test may be repeated at the PI or medically qualified designee's discretion.

Treatment Visits. At each treatment visit the PI or appropriately qualified designee, will assess the participant's continued eligibility for the study by confirming the participant still meets the entry criteria above. Urine drugs of abuse test, breath alcohol test, urine cotinine test, urinary pregnancy test (in WOCBP), COVID-19 lateral flow test and vital signs (blood pressure, temperature and heart rate) will also be conducted at each treatment visit to confirm continued eligibility. The results of all assessments will be reviewed by the PI or a medically qualified designee.

At the treatment visit participants will not be allowed any food or drinks other than those provided by the study site staff from arrival at site until departure. Safety blood samples will be taken prior to discharge on each treatment visit (approximately 24 hours post dose). Following each visit, repeat or unscheduled samples may be taken for safety reasons or for technical issues with the samples.

Participants will attend the site approximately 2 hours pre dosing on each treatment visit. Dosing will be early afternoon on each treatment visit. Participants must lie down immediately post dose until 18 hours post dose. Lunch and dinner will be provided on day 1 and breakfast and lunch will be provided on day 2 of each treatment visit. Water, decaffeinated tea and decaffeinated coffee is available ad libitum throughout the visit apart from 30 minutes prior to a scheduled ECG.

All scintigraphic images will be taken with the participant in a supine position. Dynamic imaging will be performed immediately post dosing for 10 minutes followed by static imaging until a maximum of 10 hr post-dose. Imaging will be taken at the time points specified in the SoA Blood samples for PK analysis will be taken at specified times as per the SoA until 24 hr post-dose. Safety bloods for clinical laboratory testing will be taken prior to departure on the treatment visit.

ECGs and vital sign measurements will take place at specific timepoints throughout the treatment visits as per the SoA. An acceptable time window for ECGs and vital signs is +/−15 minutes from protocol specified timepoint. Abnormal findings that the PI or medically qualified designee considers to be clinically significant, other than at screening, will be recorded as an AE or SAE.

Participants will be discharged from the study site once they have completed all study procedures and any AEs observed during the visit have been resolved or recorded for follow-up. The maximum period the participants will be resident within the study site during each treatment visit is approximately 26 hr.

Follow up Visits. Post-study follow up visit will take place 7-14 days after last attendance at the study site. This follow-up visit will consist of a repeat physical examination; assessment of vital signs; collection of blood and urine samples (the tests carried out on these samples will be the same as that of the screening visit, except COVID-19 lateral flow, cotinine and drugs of abuse which will not be conducted) and an ECG. Participants will also be questioned as to whether they experienced any AEs since the last treatment visit or have had any changes to concomitant medications. Following the follow up visit repeat or unscheduled samples may be taken for safety reasons or for technical issues with the samples.

Scintigraphic imaging. At the treatment visit, each participant will have external anatomical markers containing a maximum of 0.02 MBq ^{99m}Tc taped to the left temple and left mastoid process to allow accurate alignment of sequential images. Imaging will be taken at the following time points post dose: dynamic imaging from 0-10 mins followed by static images at 15, 30 mins, 45 minutes, 1, 2, 3, 4, 5, 6, 7, 8, 9 and 10 hours. Imaging may be stopped earlier if deemed appropriate by study staff e.g., no evidence of residual radiolabel present within the nasal cavity on two or more consecutive images.

Additional images may be acquired for safety reasons, inadequate image quality or camera technical issues at the discretion of the PI. This must be fully documented in the applicable source documentation/eCRF(s). The timing of imaging may be altered during the course of the study based on newly available data.

The actual imaging times will be recorded in the source documentation alongside the scheduled times. The actual times will be recorded in the 24-hr format. An explanation should be given for any image taken more than 2 min out with the scheduled time for images taken between 0 and 60 minutes and +/−5 minutes for images taken between 1 and 10 hours.

Scintigraphic images will be analyzed using the WebLink image analysis program. The images will be assessed by two trained analysts. The following parameters will be recorded, where appropriate:
location of radiolabel within the nasal cavity at each time point
relative amount of radiolabel within nasal cavity with time

Times will be recorded as the mid-point time between the image at which the endpoint is first observed and the previous image.

Pharmacokinetic Measurements. Samples will be used to evaluate the PK of compound 1. Each plasma sample will be divided into 2 aliquots (1 each for PK and a backup). Target characteristics for PK are: Cₘₐₓ, Tₘₐₓ, T_{lag}, AUCₗₐₛₜ, AUC_{0-inf}, K, and T_{1/2}.

At each treatment visit, blood samples will be drawn by suitably trained site staff using an indwelling cannula or by venepuncture at appropriate timepoints to allow an assessment of compound 1 concentrations in plasma as specified in the SoA. The total blood volume taken at each timepoint will be described in the study specific laboratory manual.

A pre-dose blood sample will be taken within 1 hour prior to dosing. Blood samples will then be drawn at specified intervals as follows: 15 min, 30 min, 45 min, 1, 2, 3, 4, 5, 6, 7, 8, 9, 10, 18, and 24 hours post dose.

The actual sample times (times samples taken) should be recorded alongside the expected times on the source documentation and should be entered at the time of or as soon as possible after sampling. The actual times must be recorded in 24 hr format. An explanation must be given for any blood sample taken outside of the set sampling times. An acceptable blood sampling time will be considered±5 min of the target time.

The timing of sampling may be altered during the course of the study based on newly available data (e.g., to obtain data closer to the time of peak plasma concentrations) to ensure appropriate monitoring.

The study will demonstrate safety and tolerability of the formulations containing compound 1 via nasal administration to healthy adult participants. The study will also demonstrate the location of the radiolabel within the nasal cavity with time.

### 5. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents and patent applications cited within the body of the instant specification are hereby incorporated by reference in their entirety, for all purposes. In particular, International Application No. PCT/EP2024/077436 (filed Sep. 30, 2024); U.S. Provisional Patent Application No. 63/586,254 (filed Sep. 28, 2023); UK Application No. UK Application No. 2315863.7, filed Oct. 17, 2023; and U.S. Provisional Patent Application No. 63/599,900 (filed Nov. 16, 2023) are hereby incorporated by reference in their entirety.

What is claimed is:

1. A pharmaceutical composition comprising:
2′-{[2-(4-Methoxy-phenyl)-acetylamino]-methyl}-biphenyl-2-carboxylic acid (2-pyridin-3-yl-ethyl)-amide (compound 1) or a pharmaceutically acceptable salt thereof, and
a mucoadhesive polymer.

2. The pharmaceutical composition of claim 1, wherein the composition is in the form of a liquid, such as a suspension.

3. The pharmaceutical composition of claim 1 or claim 2, wherein the composition is in the form of a microparticulate suspension.

4. The pharmaceutical composition of any one of claims 1-3, wherein compound 1 is present in the pharmaceutical composition in an amount from 0.1% (w/w) to 20% (w/w).

5. The pharmaceutical composition of claim 4, wherein compound 1 is present in the pharmaceutical composition in an amount from 3% (w/w) to 10% (w/w), such as from 6% (w/w) to 15% (w/w) or from 6% (w/w) to 10% (w/w).

6. The pharmaceutical composition of claim 4, wherein compound 1 is present in the pharmaceutical composition in an amount of about 9% (w/w).

7. The pharmaceutical composition of any one of claims 1-6, wherein the mucoadhesive polymer is selected from a polyacrylic acid derivative, cellulose derivative, natural polymer, polyvinyl pyrrolidone (PVP) polymer, dextran polymer, polyethylene oxide (PEG) polymer, thermoreversible polymer, ionic responsive polymer, copolymer of polymethyl vinyl ether and maleic anhydride, polyvinyl alcohol polymer, a salt of the foregoing, a derivative of the foregoing, and combinations thereof.

8. The pharmaceutical composition of claim 7, wherein the mucoadhesive polymer is a cellulose derivative selected from a hydroxy alkyl cellulose polymer, methyl cellulose polymer, carboxymethyl cellulose (CMC) polymer, a salt of carboxymethyl cellulose, and combinations thereof.

9. The pharmaceutical composition of claim 8, wherein the mucoadhesive polymer is a hydroxy alkyl cellulose polymer selected from hydroxypropyl methylcellulose (HPMC) and hydroxypropyl cellulose (HPC).

10. The pharmaceutical composition of claim 9, wherein the mucoadhesive polymer is a methyl cellulose polymer.

11. The pharmaceutical composition of claim 7, wherein the mucoadhesive polymer is selected from arabic gum, tragacanth gum, agar polymer, xanthan gum, guar gum, a copolymer of alginic acid and sodium alginate, a chitosan polymer, pectin, a carrageenan, pullulan polymer, modified starch, gelatin, a salt of the foregoing, a derivative of the foregoing, and combinations thereof.

12. The pharmaceutical composition of claim 11, wherein the mucoadhesive polymer is carrageenan.

13. The pharmaceutical composition of any one of claims 1-12, wherein the mucoadhesive polymer is present in the pharmaceutical composition in an amount from 0.1% (w/w) to 20% (w/w).

14. The pharmaceutical composition of claim 13, wherein the mucoadhesive polymer is present in the pharmaceutical composition in an amount from 0.3% (w/w) to 1% (w/w), such as from 0.3% (w/w) to 0.6% (w/w).

15. The pharmaceutical composition of any one of claims 1-14, wherein the pharmaceutical composition does not comprise microcrystalline cellulose.

16. The pharmaceutical composition of any one of claims 1-15, further comprising a wetting agent.

17. The pharmaceutical composition of claim 16, wherein the wetting agent is selected from a polysorbate, a fatty acid glycerol polyethylene glycol ester, a fatty acid polyethylene glycol ester, a polyethylene glycol, a glycerol ether, a cyclodextrins (such as alpha-, beta- or gamma-cyclodextrin, including alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), a reaction product of castor oil and ethylene oxide (such as polyoxyl 35 castor oil or polyoxy 40 hydrogenated castor oil), castor oil, benzalkonium chloride, edetate disodium, N-Dodecyl Beta-D-Maloside, potassium sorbate, sorbitan monolaurate, glycerin, silicon dioxide, magnesium stearate, PEG-8 laurate, poloxamer 125, poloxamer 182, poloxamer 188, poloxamer 407, polypropylene glycol 11 stearyl ether, polypropylene glycol 15 stearyl ether, laureth-4, glyceryl oleate, cetheth-20, sodium monooleate, sodium laureth-3 sulfate, sodium laroyl sarcosinate, sodium lauryl sulfate, tyloxapol, trideceth-10, amonnium lauryl sulfate, ceteareth-12, ceteareth-30, diethanolamine, diisopropanolamine, disodium laureth sulfosuccinate, disodium lauryl sulfosuccinate, docusate sodium, lauramine oxide, laureth sulfate, laureth-23, nonoxynol-9, nonoxynol-40, PEG-60 hydrogenated castor oil, polypropylene glycol, sodium laureth-2 sulfate, sodium laureth-5 sulfate, sodium methyl cocoyl taurate, Steareth-20, Steareth-21, Steareth-40, and combinations thereof.

18. The pharmaceutical composition of claim 17, wherein the wetting agent is selected from a polysorbate, a reaction product of castor oil and ethylene oxide, and a combination thereof.

19. The pharmaceutical composition of claim 18, wherein the wetting agent is a polysorbate, wherein the polysorbate is polysorbate 80.

20. The pharmaceutical composition of claim 18, wherein the wetting agent is a reaction product of castor oil and ethylene oxide, such as polyoxyl 35 castor oil or polyoxyl 40 hydrogenated castor oil.

21. The pharmaceutical composition of any one of claims 16-20, wherein the wetting agent is present in the pharmaceutical composition in an amount from 0.1% (w/w) to 5.0% (w/w).

22. The pharmaceutical composition of claim 21, wherein the wetting agent is present in the pharmaceutical composition in an amount from 0.1% (w/w) to 1.0% (w/w), such as from 0.3% (w/w) to 0.6% (w/w).

23. The pharmaceutical composition of any one of claims 1-22, further comprising a tonicity modifier.

24. The pharmaceutical composition of claim 16, wherein the tonicity modifier is selected from dextrose, lactose, sodium chloride, calcium chloride, magnesium chloride, potassium chloride, sorbitol, sucrose, mannitol, trehalose, raffinose, polyethylene glycol, hydroxyethyl starch, glycine, glycerin, sodium acetate, sodium sulfate, and combinations thereof.

25. The pharmaceutical composition of claim 24, wherein the tonicity modifier is selected from sorbitol, mannitol, and a combination thereof.

26. The pharmaceutical composition of any one of claims 16-25, wherein the tonicity modifier is present in the pharmaceutical composition an amount from 0.1% (w/w) to 10% (w/w), such as from 0.1% (w/w) to 5% (w/w).

27. The pharmaceutical composition of claim 26, wherein the tonicity modifier is present in the pharmaceutical composition in an amount from 1% (w/w) to 3% (w/w).

28. The pharmaceutical composition of any one of claims 1-27, further comprising a preservative.

29. The pharmaceutical composition of claim 28, wherein the preservative is selected from chlorhexidine gluconate, phenyl ethyl alcohol, 1-phenoxyethanol, benzyl alcohol, sorbic acid, thimerosal, phenylmercuric acetate, a benzoate (such as sodium benzoate), a paraben (such as methyl paraben, propyl paraben, and butyl paraben), a sorbate, benzalkonium chloride, chlorobutanol, sodium metabisulfate, trisodium citrate dihydrate, boric acid, calcium acetate, dehydroacetic acid, diazolidinyl urea, dichlorobenzyl alcohol, imidurea, methyl salicyclate, methylchloroisothiazolinone, methylchloroisothiazolinone/methylchloroisothiazolinone mixture, propionic acid, sodium sulfite, a salt of the foregoing, and combinations thereof.

30. The pharmaceutical composition of claim 29, wherein the preservative is potassium sorbate.

31. The pharmaceutical composition of any one of claims 28-30, wherein the preservative is present in the pharmaceutical composition in an amount from 0.01% (w/w) to 2% (w/w).

32. The pharmaceutical composition of claim 31, wherein the preservative is present in the pharmaceutical composition in an amount from 0.05% (w/w) to 0.15% (w/w).

33. The pharmaceutical composition of any one of claims 1-32, further comprising an aqueous vehicle.

34. The pharmaceutical composition of claim 33, wherein the aqueous vehicle comprises deionized water, saline, phosphate buffer, citrate buffer, malate buffer, tartrate buffer, balanced salt solution, salts of organic acids, combinations of organic acids and salts of organic acids (such as tribasic sodium citrate and citric acid, malic acid and sodium malate, and potassium sodium tartrate and tartaric acid), acetic acid, hydrochloric acid, potassium phosphate (monobasic), sodium phosphate dibasic (heptahydrate), sodium phosphate monobasic (anhydrous/monohydrate), sodium hydroxide, potassium hydroxide, sodium gluconolactone, lactic acid, nitric acid, sodium acetate, sodium lactate, tromethamine, ammonia, citric acid, calcium acetate, diethanolamine, diisopropanolamine, phosphoric acid, potassium citrate, potassium hydroxide, succinic acid, sulfuric acid, arginine hydrochloride, or a combination thereof.

35. The pharmaceutical composition of claim 34, wherein the aqueous vehicle comprises citrate-phosphate buffer.

36. The pharmaceutical composition of any one of claims 33-35, wherein the pharmaceutical composition comprises aqueous vehicle in an amount from 85% (w/w) to 95% (w/w).

37. The pharmaceutical composition of claim 36, wherein the pharmaceutical composition comprises aqueous vehicle in an amount from 85% (w/w) to 90% (w/w).

38. The pharmaceutical composition of any one of claims 1-37, further comprising a chelating agent.

39. The pharmaceutical composition of claim 38, wherein the chelating agent is selected from ethylenediaminetetraacetic acid (EDTA), citric acid monohydrate, trisodium HEDTA, edetate calcium disodium, edetate sodium, edetate trisodium, edetic acid, pentasodium pentetate, sodium acetate, and combinations thereof.

40. The pharmaceutical composition of claim 39, wherein the chelating agent is EDTA.

41. The pharmaceutical composition of any one of claims 38-40, wherein the pharmaceutical composition comprises the chelating agent in an amount from 0.01% (w/w) to 2% (w/w).

42. The pharmaceutical composition of any one of claims 1-37, wherein the pharmaceutical composition comprises:
a. from 0.1% (w/w) to 20% (w/w) compound 1, such as 6% (w/w) to 20% (w/w) or 6% (w/w) to 12% (w/w);
b. from 0.1% (w/w) to 20% (w/w) mucoadhesive polymer;
c. from 0.1% (w/w) to 10% (w/w) tonicity modifier;
d. from 0.1% (w/w) to 5% (w/w) wetting agent;
e. from 0.01% (w/w) to 2% (w/w) preservative; and
f. from 85% (w/w) to 95% (w/w) aqueous vehicle.

43. The pharmaceutical composition of claim 42, wherein the pharmaceutical composition comprises:
a. from 0.3% (w/w) to 0.6% (w/w) mucoadhesive polymer,
b. from 1% (w/w) to 3% (w/w) tonicity modifier,
c. from 0.3% (w/w) to 0.6% (w/w) (w/w) wetting agent,
d. from 0.05% (w/w) to 0.15% (w/w) preservative; and
e. from 85% (w/w) to 90% (w/w) aqueous vehicle.

44. The pharmaceutical composition of claim 42 or 43, wherein the mucoadhesive polymer is methyl cellulose, the tonicity modifier is mannitol, the wetting agent is polyoxyl 40 hydrogenated castor oil, the preservative is potassium sorbate, and the aqueous vehicle is citrate-phosphate buffer.

45. The pharmaceutical composition of claim 42 or 43, wherein the mucoadhesive polymer is HPMC, the tonicity modifier is sorbitol, the wetting agent is polyoxyl 35 castor oil, the antimicrobial preservative is potassium sorbate, and the aqueous vehicle is citrate-phosphate buffer.

46. The pharmaceutical composition of claim 42 or 43, wherein the mucoadhesive polymer is carrageenan, the tonicity modifier is sorbitol, the wetting agent is polyoxyl 35 castor oil, the antimicrobial preservative is potassium sorbate, and the aqueous vehicle is citrate-phosphate buffer.

47. The pharmaceutical composition of claim 46, further comprising EDTA in an amount from 0.01% (w/w) to 2% (w/w).

48. The pharmaceutical composition of any one of claims 1-47, wherein the pharmaceutical composition is in the form of a microparticulate suspension and comprises less than 0.1% (w/w) of compound 1 in solution, such as from 0.001% (w/w) to 0.1 (w/w) compound 1 in solution.

49. The pharmaceutical composition of any one of claims 1-48, wherein the pharmaceutical composition has an osmolality from 200 mOsm/kg to 500 mOsm/kg, such as from 250 mOsm/kg to 350 mOsm/kg.

50. The pharmaceutical composition of any one of claims 1-49, wherein the pharmaceutical composition has an apparent pH from 4 to 9, such as from 5.5 to 6.5 or from 6 to 6.5.

51. The pharmaceutical composition of any one of claims 1-50, wherein the particle size distribution (D₁₀) of compound 1 is from 0.1 μm to 5 μm, such as from 0.1 μm to 1.5 μm or from 0.1 μm to 3 μm.

52. The pharmaceutical composition of any one of claims 1-51, wherein the pharmaceutical particle size distribution (D₅₀) of compound 1 is from 2 μm to 15 μm, such as from 2 μm to 5 μm or from 5 μm to 15 μm.

53. The pharmaceutical composition of any one of claims 1-52, wherein the particle size distribution (D₉₀) of compound 1 is from 4 μm to 50 μm, such as from 5 μm to 35 μm, from 5 μm to 15 μm, or from 10 μm to 35 μm.

54. The pharmaceutical composition of any one of claims 1-49, wherein the particle size distribution of compound 1 is:
(a) D₁₀ from 0.1 to 1.5 μm, D₅₀ from 2 to 5 μm, and D₉₀ from 5 to 10 μm; or
(b) D₁₀ from 0.1 to 3 μm, D₅₀ from 5 to 15 μm, and D₉₀ from 10 to 35 μm.

55. The pharmaceutical composition of any one of claims 1-54, wherein the pharmaceutical composition has a viscosity from 1 cP to 100 cP, such as from 30 cP to 50 cP.

56. The pharmaceutical composition of any one of claims 1-55, wherein the pharmaceutical exhibits a shear-thinning or Newtonian rheology.

57. The pharmaceutical composition of any one of claims 1-56, wherein the pharmaceutical composition has a surface tension from 40 mN/m to 50 mN/m.

58. The pharmaceutical composition of any one of claims 1-57, wherein the pharmaceutical composition is stable when stored at 25° C. or 40° C. for at least 2 weeks as determined by one or more of the following: (i) compound 1 recovery, (ii) macroscopic appearance, (iii) microscopic appearance, (iv) apparent pH, (v) osmolality, (vi) particle size distribution (D₉₀), (vii) droplet size, and (viii) Raman analysis.

59. The composition of any one of claim 58, wherein the composition is stable when stored at 25° C. or 40° C. for at least 4 weeks as determined by one or more of the following: (i) compound 1 recovery, (ii) macroscopic appearance, (iii) microscopic appearance, (iv) apparent pH, (v) osmolality, (vi) particle size distribution (D₉₀), and (vii) Raman analysis.

60. A nasal delivery device comprising the pharmaceutical composition of any one of claims 1-59 and a device suitable for intranasal delivery of the pharmaceutical composition to a subject.

61. The nasal delivery device of claim 60, wherein the device comprises a nosepiece for fitting to a nostril of the subject and an actuation mechanism, wherein the nosepiece includes a nozzle through which the composition is delivered as a spray into the nasal airway of the subject upon actuation of the actuation mechanism.

62. The nasal delivery device of claim 60 or 61, wherein the device comprises a reservoir 1 containing the pharmaceutical composition and a pump 10, said pump 10 being assembled on said reservoir 1 by means of a fastener ring 5, wherein the fluid pump 10 comprises a pump body 11, a piston 20 that slides in leaktight manner in said pump body 11 between a rest position and an actuated position, said piston 20 being secured to an actuator rod 30 that extends axially out from said pump body 11, a ferrule 40 being mounted on the top edge 12 of said pump body 11, said ferrule 40 defining the rest position of said piston 20, the pump being characterized in that said ferrule 40 co-operates in leaktight manner with the actuator rod 30 while the actuator rod 30 is moving between the rest position and an intermediate position, and said ferrule 40 co-operating in non-leaktight manner with the actuator rod 30 while the actuator rod 30 is moving between said intermediate position and the actuated position, so as to enable the pump 10 to be vented, said piston 20 not being in contact with said ferrule 40, when in the rest position.

63. The nasal delivery device of claims 61 or 62, wherein a volume of 50 μl to 150 μl of the pharmaceutical composition is delivered intranasally to the subject per actuation of the device.

64. The nasal delivery device of claim 63, wherein the device provides a jet spray of the pharmaceutical composition upon actuation of the device.

65. The nasal delivery device of claim 64, wherein the jet spray comprises droplets having a droplet size from 100 μm to 1,000 μm, such as from 700 μm to 1,000 μm.

66. The nasal delivery device of claim 63, wherein the device provides a mist spray of the pharmaceutical composition upon actuation of the device.

67. The nasal delivery device of claim 66, wherein the mist spray comprises droplets having a droplet size from 50 μm to 1,000 μm, such as from about 80 μm to about 100 μm, or from 80 μm to 90 μm.

68. The nasal delivery device of any one of claims 60-67, wherein the device delivers from 85 mg to 130 mg of the pharmaceutical composition per actuation of the device, such as from 100 mg to 120 mg or from 105 mg to 120 mg.

69. The nasal delivery device of any one of claims 60-68, wherein the device delivers from 50 μl to 300 μl of the pharmaceutical composition per actuation of the device, such as from 150 μl to 250 μl.

70. A method for treating obstructive sleep apnea in a subject in need thereof, the method comprising administering an effective amount of the pharmaceutical composition of any one of claims 1-59 to the subject intranasally.

71. The method of claim 70, wherein the pharmaceutical composition is administered prior to sleep, such as from 5 to 120 minutes prior to sleep or from 10 to 30 minutes prior to sleep.

72. The method of claim 70 or 71, wherein the pharmaceutical composition is administered while the subject is in an upright position or the subject's head is elevated up to 450 to the horizontal.

73. The method of claim 72, wherein the subject assumes a post-administration position that is supine or with a head elevation of 45° or less to the horizontal following administration of the pharmaceutical composition.

74. The method of claim 73, wherein the subject assumes the post-administration position within 20 minutes following administration of the pharmaceutical composition, such as within 10 minutes.

75. The method of claim 73 or 74, wherein the subject maintains the post-administration position for at least 4 hours following administration of the pharmaceutical composition, such as 4 to 10 hours or at least 8 hours.

76. The method of any one of claims 70-75, wherein a total dose of from 0.1 mg to 100 mg compound 1 is administered to the subject, such as from 0.1 mg to 30 mg or from 10 mg to 20 mg.

77. The method of any one of claims 70-76, wherein upper-airway collapsibility of the subject is inhibited for at least 4 hours following administration and during sleep as measured by a reduction in pharyngeal critical closing pressure (P_{crit}) compared to P_{crit} measured for the same subject in the absence of administration of the composition, such as from 4 to 10 hours.

78. The method of claim 77, wherein P_{crit} is reduced by at least 2 cm H₂O for at least 4 hours, such as from 2 to 10 cm H₂O.

79. The method of claim 77 or 78, wherein P_{crit} is reduced for at least 8 hours.

80. The method of any one of claims 70-79, wherein the subject does not experience reverse sneezing.

81. The method of any one of claims 70-79, wherein the pharmaceutical composition is administered via the nasal delivery device of any one of claims 60-69.

82. The method of claim 81, wherein one or more priming sprays of the nasal delivery device are performed prior to administration to the subject, such as four priming sprays.

83. The method of claim 81 or 82, wherein the pharmaceutical composition is administered into a single nostril by actuation of the nasal delivery device to provide the total dose of compound 1 to the subject.

84. The method of claim 81 or 82, wherein the pharmaceutical composition is administered into each nostril by actuation of the nasal delivery device to provide the total dose of compound 1 to the subject.
